(19) United States
(12) Patent Application Publication
(10) Pub. No.: US 2021/0101946 A1
(43) Pub. Date: Apr. 8, 2021

(54) HIGH MOLECULAR WEIGHT SILK FIBROIN AND USES THEREOF

(71) Applicant: Trustees of Tufts College, Medford, MA (US)

(72) Inventors: Tim Jia-Ching Lo, Taoyuan (TW); Gary G. Leisk, Wilmington, MA (US); Benjamin Partlow, Marlborough, MA (US); Fiorenzo Omenetto, Lexington, MA (US); David L. Kaplan, Concord, MA (US); Jonathan A. Kluge, Southborough, MA (US); Matthew A. Kluge, Southborough, MA (US)

(21) Appl. No.: 16/998,966

(22) Filed: Aug. 20, 2020

Related U.S. Application Data
(63) Continuation of application No. 14/413,312, filed on Jan. 7, 2015, now abandoned.
(60) Provisional application No. 61/761,533, filed on Feb. 6, 2013.
(60) Provisional application No. 61/669,405, filed on Jul. 9, 2012.

(51) Int. Cl. C07K 14/435; A61L 27/56; A61L 27/36; A61L 27/54; A61L 27/50; A61L 27/52; A61L 27/58; A61K 33/42; A61K 47/42; A61L 31/04; C08L 89/00

(52) U.S. Cl. CPC C07K 14/43586; A61L 2430/22; A61L 27/3604; A61L 27/54; A61L 27/507; A61L 27/52; A61L 27/58; A61K 33/42; A61K 47/42; A61L 31/046; A61L 31/047; C08L 89/00; Y10T 428/13; Y10T 428/249921; Y10T 442/10; Y10T 442/60; A61L 2430/02; A61L 2430/04; A61L 27/56

(57) ABSTRACT

Provided herein relates to high molecular weight silk-based materials, compositions comprising the same, and processes of preparing the same. The silk-based materials produced from high molecular weight silk can be used in various applications ranging from biomedical applications such as tissue engineering scaffolds to construction applications. In some embodiments, the high molecular weight silk can be used to produce high strength silk-based materials. In some embodiments, the high molecular weight silk can be used to produce silk-based materials that are mechanically strong with tunable degradation properties.

### CROSS REFERENCE TO RELATED APPLICATIONS

This application claims the benefit under 35 U.S.C. § 119(e) of U.S. Provisional Application Nos. 61/669,405 filed Jul. 9, 2012 and 61/761,533 filed Feb. 6, 2013, the content of each of which is incorporated herein by reference in its entirety.

### GOVERNMENT SUPPORT

This invention was made with government support under P41 EB002520 awarded by the National Institutes of Health (NIH) and FA9550-10-1-0172 awarded by Air Force of Scientific Research (AFOSR). The government has certain rights in the invention.

### TECHNICAL FIELD

The present invention relates to silk fibroin-based materials, processes of making the same and uses of the same.

### BACKGROUND

Silk from the domesticated silkworm, Bombyx mori, is a tough and versatile material that has been used as a cloth and sutures. Silk has also been discussed to be used in a regenerated form as scaffolds for tissue engineering, sustained drug delivery and technological applications (See, e.g., Vepari, C. and Kaplan, D. L., Progress in Polymer Science, 2007, 32: 991-1007). In native silk fibers, the amino acid sequence of the primary structural component of the silk protein, fibroin, can allow for close packing and highly aligned molecules that imbue the silk with desirable mechanical properties, e.g., providing high tensile strength with ductility and toughness. The natural silk fiber can rival synthetic polymer fibers with regards to its combination of strength, extensibility and toughness (Fu, C., et al., Chem. Comm., 2009 (43): 6515-6529).

While silk in its native fiber form has been discussed to be used in biomedical engineering, for example, for replacing and strengthening connective tissues including ligaments and tendons and in the closure of wounds, use of native silk fibers to produce other forms of constructs such as a foam can be challenging. In contrast, silk solutions that are produced by solubilizing silk cocoons can be reconstituted to create myriad constructs including, e.g., fibers, films, foams and sponges. While regenerated silk fibroin has been discussed as a biocompatible material for use in biomedical engineering, it can be desirable to tune the mechanical properties of constructs made from regenerated silk fibroin depending on the certain applications. Hence, there is an unmet need for new types of regenerated silk fibroin materials with enhanced mechanical strength and tunable degradation profiles.

### SUMMARY

While silk fibroin present in native silk exhibits robust mechanical properties, sericin removal is desired in the context of biomedical applications due to its implication in inflammatory response. Accordingly, there is an unmet need for isolating the substantially sericin-removed silk fibroin from native silk while preserving robust mechanical properties of natural silk fibroin for the development of new types of silk-based materials with enhanced mechanical properties.

Sericin is typically removed from native silk through an extended boiling process (e.g., about 20-30 minutes at boiling temperatures) under basic conditions. The inventors have demonstrated inter alia that milder degumming processes (e.g., heating silk cocoons at a temperature of about 90° C. or higher for less than 5 minutes or at a lower temperature (e.g., as low as about 60° C.-70° C.) for a longer period of time (e.g., about 30 minutes or longer) can not only reduce degradation of silk fibroin protein chains and thus generate silk fibroin of higher average molecular weights, but can also substantially remove sericin from native silk fibers. A typical degumming process generally involves heating silk cocoons at a temperature of at least about 90° C. for at least about 20-30 minutes. Accordingly, the inventors have discovered a degumming condition at which surprisingly, a substantial amount of sericin can be removed from native silk fibers to yield a higher molecular weight silk fibroin solution than what is typically achieved. This is the first example of a reconstituted substantially sericin-free silk fibroin solution with a high molecular weight range, which can be subsequently used to form different silk fibroin articles as described herein. Further, the inventors have discovered enhanced mechanical properties of silk fibroin-based materials made from the higher molecular weight silk fibroin. In particular, high molecular weight silk fibroin can be used at a low concentration, for example, as low as 0.5% w/v silk fibroin or lower, to form a mechanically robust silk fibroin-based scaffold with desirable degradation properties. Accordingly, embodiments of various aspects described herein relate to novel compositions comprising a silk-based material of high molecular weight silk fibroin, methods of making the same and uses of the same.

One aspect provided herein is a composition comprising a solid-state silk fibroin, wherein the silk fibroin has an average molecular weight of at least about 200 kDa, and wherein no more than 30% of the silk fibroin has a molecular weight of less than 100 kDa. In some embodiments, the solid-state silk fibroin can have a sericin content of less than 5% or lower.

The solid-state silk fibroin can be present in any form. In some embodiments, the solid-state silk fibroin can be in a form selected from the group consisting of a film, a sheet, a gel or hydro gel, a mesh, a mat, a non-woven mat, a fabric, a scaffold, a tube, a slab or block, a fiber, a particle, powder, a 3-dimensional construct, an implant, a foam or a sponge, a needle, a lyophilized article, and any combinations thereof.

In some embodiments, the composition can further comprise an additive. The additive can be incorporated into the solid-state silk fibroin. Non-limiting examples of the additive include biocompatible polymers; plasticizers; stimulus-responsive agents; small organic or inorganic molecules; saccharides; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; glycogens or other sugars; immunogens; antigens; an extract made from biological materials such as bacteria, plants, fungi, or animal cells; animal tissues; naturally occurring or synthetic compositions; and any combinations thereof.

The additive can be in any form. For example, the additive can be in a form selected from the group consisting of a particle, a fiber, a tube, a film, a gel, a mesh, a mat, a non-woven mat, a powder, and any combinations thereof. In some embodiments, the additive can comprise a particle, e.g., a nanoparticle or a microparticle.

In some embodiments, the additive can comprise a calcium phosphate (CaP) material, e.g., apatite. In some embodiments, the additive can comprise a silk material, e.g., silk particles, silk fibers, micro-sized silk fibers, and unprocessed silk fibers.

In some embodiments, the composition can further comprise an active agent. The active agent can be incorporated into the solid-state silk fibroin. In one embodiment, the active agent can comprise a therapeutic agent.

In some embodiments, the composition can comprise from about 0.1% (w/w) to about 99% (w/w) of the additive agent and/or active agent.

Another aspect provided herein relates to a silk fibroin article comprising one or more embodiments of the composition described herein. The article can be in a form selected from the group consisting of a film, a sheet, a gel or hydrogel, a mesh, a mat, a non-woven mat, a fabric, a scaffold, a tube, a slab or block, a fiber, a particle, a powder, a 3-dimensional construct, an implant, a foam or a sponge, a needle, a lyophilized article, and any combinations thereof. In some embodiments, the article can include, but are not limited to, bioresorbable implants, tissue scaffolds, sutures, reinforcement materials, medical devices, coatings, construction materials, wound dressing, tissue sealants, fabrics, textile products, and any combinations thereof.

A further aspect provided herein is a method of producing a silk fibroin article, e.g., but not limited to, a film, a sheet, a gel or hydrogel, a mesh, a mat, a non-woven mat, a fabric, a scaffold, a tube, a slab or block, a fiber, a particle, powder, a 3-dimensional construct, an implant, a foam or a sponge, a needle, a lyophilized article, and any combinations thereof. The method comprises: (i) providing a composition comprising silk fibroin having an average molecular weight of at least 200 kDa, and wherein no more than 30% of the silk fibroin has a molecular weight of less than 100 kDa; and (ii) forming the silk fibroin article from the composition.

In some embodiments, the high molecular silk fibroin can be produced by a process comprising degumming silk cocoons at a temperature in a range of about 60° C. to about 90° C. Accordingly, another aspect provided herein is a method of producing a silk fibroin article comprising: (i) providing a composition comprising silk fibroin, wherein the silk fibroin is produced by degumming silk cocoons at a temperature in a range of about 60° C. to about 90° C.; and (ii) forming the silk fibroin article from the composition. In one embodiment, the silk cocoons can be degummed for at least about 30 minutes.

In some embodiments, the silk fibroin can be produced by degumming silk cocoons for no more than 15 minutes at a temperature of at least about 90° C. Thus, a further aspect provided herein is a method of producing a silk fibroin article comprising: (i) providing a composition comprising silk fibroin, wherein the silk fibroin is produced by degumming silk cocoons for no more than 15 minutes at a temperature of at least about 90° C.; and (ii) forming the silk fibroin article from the composition.

In some embodiments of this aspect and other aspects described herein, the composition comprising high molecular weight silk fibroin can be provided as a solution or powder.

In some embodiments of this aspect and other aspects described herein, the silk fibroin article can be formed from the composition by a process selected from the group consisting of gel spinning, lyophilization, casting, molding, electrospinning, machining, wet-spinning, dry-spinning, milling, spraying, phase separation, template-assisted assembly, rolling, compaction, and any combinations thereof.

In some embodiments of this aspect and other aspects described herein, the method can further comprise subjecting the silk fibroin article to a post-treatment. In one embodiment, the post-treatment can comprise steam drawing. In some embodiments, the post-treatment can induce a conformational change in the silk fibroin in the article. Exemplary methods for inducing a conformational change in the silk fibroin can comprise one or more of lyophilization, water annealing, water vapor annealing, alcohol immersion, sonication, shear stress, electrogelation, pH reduction, salt addition, air-drying, electrospinning, stretching, or any combination thereof.

In some embodiments, the silk fibroin article can further comprise an additive as described herein. The additive can be incorporated into the silk fibroin article during or after its formation. In some embodiments, the silk fibroin article can further comprise an active agent. The active agent can be incorporated into the silk fibroin article during or after its formation.

In some embodiments, the composition can comprise from about 0.1% (w/w) to about 99% (w/w) of the additive and/or active agent.

A still another aspect provided herein is a method of substantially removing sericin from silk cocoons (e.g., to yield high molecular weight silk fibroin) comprising: (i) degumming silk cocoons for no more than 15 minutes (or no more than 10 minutes, or no more than 5 minutes) at a temperature of at least about 90° C.; or (ii) degumming silk cocoons for at least about 30 minutes at a temperature in a range of about 60° C. to about 90° C. In one embodiment, the silk cocoons can be degummed for less than 5 minutes at a temperature of at least about 90° C. or higher.

A yet another aspect provided herein is a composition comprising silk fibroin (e.g., high molecular weight silk fibroin), wherein the solution is substantially free of sericin, and wherein sericin is removed by (i) degumming silk cocoons for no more than 15 minutes (or no more than 10 minutes, or no more than 5 minutes) at a temperature of at least about 90° C.; or (ii) degumming silk cocoons for at least about 30 minutes at a temperature in a range of about 60° C. to about 90° C. In one embodiment, the silk cocoons can be degummed for less than 5 minutes at a temperature of at least about 90° C. or higher.

### BRIEF DESCRIPTION OF THE DRAWINGS

The patent or application file contains at least one drawing executed in color. Copies of this patent or patent application publication with color drawing(s) will be provided by the Office upon request.

FIG. 1 shows mass loss during degumming of Japanese cocoons in boiling or sub-boiling (e.g., —70° C.) conditions in ˜0.02M sodium carbonate (Na₂CO₃) solution for various durations. Mass loss can then be used to calculate residual sericin content, using an original value of 26.3% of the starting mass as sericin.

FIGS. 2A-2B are images of SDS-PAGE gel for silk fibroin produced by degumming silk cocoons in boiling or sub-boiling (e.g., ˜70° C.) in 0.02M Na₂CO₃ solution for various durations. In FIG. 2A, lanes 1-8 represent about 2.5, 5, 7.5, 10, 15, 20, 30, 60 minutes boiled (mb), respectively. In FIG. 2B, lanes 1-4 represent about 60, 90, 120 and 150 minutes immersion in 70° C. degumming solution, respectively.

FIGS. 3A-3B show the molecular weight distribution of silk in degummed silk solutions depending on the degumming time and temperature. FIG. 3A shows the normalized pixel intensity. FIG. 3B shows the percentage of each molecular weight group for different degumming conditions.

FIG. 4 shows the Bingham plastic viscosity of degummed silk solutions as a function of degumming time and temperature.

FIG. 5 shows the rheological properties of different degummed silk solutions. Storage modulus (G′) and loss modulus (G″) are shown in solid and open markers, respectively.

FIG. 6 shows the rheological data for native and reconstituted silk solutions. Storage modulus (G′) and loss modulus (G″) are marked respectively. The data on native silk is adapted from Holland, et al. 2007 (Holland, C., et al., Polymer, 2007, 48 (12): 3388-3392).

FIG. 7A-7B show silk films made from silk fibroin with short degumming time. FIG. 7A shows a silk film after removal from an acrylic base sheet. FIG. 7B shows a silk film after removal from a diffraction grating.

FIGS. 8A-8B are images showing steam drawing of a silk film strip and subsequent tensile testing in a fixture. FIG. 8A shows that a silk film strip is pulled while being exposed to a steam jet generated by heating beaker on hot plate with custom fitted top.

FIG. 8B shows a silk sample with tape applied and ready for mounting in the tensile testing fixture.

FIG. 9 plots the draw ratio of ˜6.2 mm wide silk film strips as a function of degumming condition. Significant differences were found between the 30 mb and 60 mb groups and all other conditions, p<0.01.

FIGS. 10A-10B show linear elastic modulus of silk films in as cast and steam drawn states for (A) films of different degumming times at boiling temperature, and (B) films of different degumming times at 70° C.

FIGS. 11A-11B show maximum extensibility of silk films in as cast and steam drawn states for (FIG. 11A) films of different degumming times at boiling temperature, and (FIG. 11B) films of different degumming times at ˜70° C.

FIGS. 12A-12B show ultimate tensile strength of silk films in as cast and steam drawn states for (FIG. 12A) films of different degumming times at boiling temperature, and (FIG. 12B) films of different degumming times at ˜70° C.

FIG. 13 shows representative material behavior of as cast and steam drawn silk films. As cast film shows brittle behavior while steam drawn exhibits significantly enhanced ductility.

FIGS. 14A-14C show the amide I band of FTIR spectra of different silk films casted from different degummed solutions, with or without post treatments. FIG. 14A shows that degumming time does not result in detectable conformation differences in un-annealed silk films. FIG. 14B shows the FTIR spectra of 5 mb and 60 mb cast films subjected to water annealing and methanol treatments. Spectra show characteristic shift to β-sheet (vertical line at 1620 cm⁻¹) with post treatments, but inter-group differences are not apparent. FIG. 14C shows the FTIR spectra of different silk films casted from differently degummed solutions and steam drawn. Spectra show shift toward β-sheet with slightly inhibited shift for 20 mb and 60 mb samples. The 60 mb as-cast film included for comparison.

FIG. 15 shows the representative stress-strain response of native silk fibers, steam drawn silk films and as cast films.

FIGS. 16A-16E are schematic representations of example mechanisms and kinetics of self-assembly for differently degummed silk fibroin solutions. FIG. 16A shows a hydrophobicity pattern in fibroin chain. FIG. 16B shows a mechanism of self-assembly for native silks. Protein chains assemble into micelles, for globules and are sheared to produce fibers (Jin, H. J., Kaplan, D. L., Nature, 2003, 424 (6952):1057-1061). FIG. 16C shows that gently degummed silks can retain residual entanglements formed during initial fiber formation. Without wishing to be bound by theory, entanglements can inhibit micelle and globule formation, and prevent efficient extensional shear. FIG. 16D shows that silks under traditional degumming conditions can have all residual entanglements removed, but can have shortened chain lengths and fewer hydrophilic tails than native chains, allowing native like micelle and globule formation. Under shear, the inter-micelle hydrophilic associations are not as strong, allowing extensional flow with higher extensibility, but lower tensile strength. FIG. 16E shows that aggressively degummed silk fibroin can result in significantly shorter chain lengths and a lower molecular weight distribution. In some embodiments, aggressively degummed silk of shorter chain lengths can have no remaining hydrophilic tails. In these embodiments, micelle formation and globule formation can occur, but have ineffective shielding of the hydrophobic core. These short chains and weak micelle associations can limit extensibility and/or strength under shear.

FIGS. 17A-17F depict an exemplary process to generate silk fibers from high molecular weight silk fibroin. FIG. 17A shows formation of silk gel by electrogelation (egel) using a ˜10-min degummed silk solution. FIG. 17B shows heating of egel with a heat gun. FIG. 17C shows fast ejection of the heated egel into a pure water bath. FIG. 17D shows a wet-spun silk fiber; and FIG. 17E shows the silk fiber after drawing out of the bath. FIG. 17F shows a regenerated silk fiber with multiple tied knots.

FIGS. 18A-18B show the mechanical properties of ˜2% wt/v autoclaved silk fibroin scaffolds as a function of boiling time (5-60 min).

FIG. 19 is a set of photographs showing autoclaved silk fibroin scaffolds made from about 5-60 mb (mins boiling) silk fibroin at about 0.5-4% wt/v silk concentration.

FIG. 20A is a set of SEM micrographs showing pore and lamellae morphology of autoclaved silk scaffolds made from ˜5 mb and ˜30 mb silk at ˜0.5% wt/v concentration. FIG. 20B is a set of SEM micrographs showing pore and lamellae morphology of autoclaved silk fibroin scaffolds made from ˜5 mb silk at about 0.5-4% wt/v concentration. The zoomed-in micrographs show that the lamellae wall thickness decreases as the concentration decreases.

FIGS. 21A-21C shows degradation of ˜2% wt/v silk scaffolds made from silk degummed for different boiling durations (˜5-60 min) followed by different post-treatments that can induce 0 sheet content (e.g., 2-hour water annealing, overnight (o/n) water annealing and autoclaved) in the presence of 1 U/ml Protease XIV. FIG. 21D-21F shows degradation of ˜5 mb silk scaffolds at different concentrations (˜0.5-4% wt/v) with β-sheet contents formed by different methods (e.g., 2-hour water annealing, o/n water annealing and autoclaved) in the presence of 1 U/ml Protease XIV.

FIGS. 22A-22B show various silk fibroin articles produced from high molecular weight silk fibroin in accordance with some embodiments described herein. FIG. 22A shows a silk-based coffee cup. FIG. 22B shows a silk foam with gold nanoparticles embedded. FIG. 22C shows a silk foam-based skull. FIG. 22D shows a silk foam-based breast implant concept.

FIGS. 23A-23D is a set of photographs showing raw egg components suspended in silk foam. FIG. 23A shows an egg yolk in silk foam. FIG. 23B shows egg white in silk foam. FIG. 23C shows egg yolk/silk foam under loading, and FIG. 23D shows egg white/silk foam under loading.

FIGS. 24A-24D is a set of photographs showing integrated raw eggs stabilized with silk. FIG. 24A shows a platinum-cured silicone mold in oven. FIG. 24B shows a hard-boiled egg used as a mold positive. FIG. 24C shows a final mold for creating a foam in egg yolk geometry. FIG. 24D shows a finished silk-stabilized foam egg.

FIGS. 25A-25C show subcutaneous implantation of an exemplary silk foam in an animal. FIG. 25A shows a silk foam construct. FIG. 25B shows a silk foam injector loaded with a silk foam. FIG. 25C shows injection of a silk foam using the silk foam injector into an animal.

FIG. 26A is a bar graph showing effects of boiling times of a silk solution on viscosity. Silk solutions prepared using increasing boiling times decrease in viscosity (5, 10, and 30 minute boil [5, 10, 30 mb] shown in the figure), as measured by a Brookfield™DV-II+Pro viscometer, a trend that scales with increasing solution concentration. The dotted line indicates the spinnable viscosity threshold. FIG. 26B is an image showing end-to-end anastomosis of an interposed silk gel-spun vascular graft formed from 20 mb solution. Grids=1 mm spaces.

FIGS. 27A-27B show experimental data on effects of silk solution boiling time on tube structure and degradability. In FIG. 27A, tubes formed from 5 mb, 10 mb, 20 mb, 30 mb, (14%, 16%, 26%, 34% w/v concentrations, respectively) showed different pore structures after lyophilization. Scale bars 200 μm for cross-sectional images. Inset shows the inner lumen of each tube (inset scale bar=500 μm). Layered composite tube designs can be generated to fine-tune properties, here showing an inner layer of 30 mb covered by an outer 20 mb layer (separated by the dotted line). In FIG. 27B, subject to Protease XIV enzyme exposure (or a PBS control) for 14 days, tube samples showed unique degradation profiles depending on boil time (10 mg each, constant orbital shaking, replacement every 2-3 days). The 5 mb group was the fastest to degrade, likely due to rapid fluid transport through the large pores.

FIG. 28 shows a set of histological cross-sections of silk tubes produced by some embodiments of the method described herein. (Left) H&E stain, (Mid-Left) trichrome stain, (Mid-Right) and elastic stain. Native vessel proximal to the graft with elastic stain (Right). Upper row 50×, lower 200× magnification. Scale bars representative.

FIG. 29 is a set of images showing histology of silk fibroin tube graft 2 weeks and 4 weeks post-implantation. Full cross-sections were taken at 2 weeks and 4 weeks post-implant for the native aorta (section 1, close to the interface with the silk tube) and at two different positions along the implanted silk tube graft (section 2 and section 3), as shown on the schematics. Blood flow is from left to right. Adjacent histological sections were stained for hematoxilin and eosin (H&E), smooth muscle actin (SMA) and Factor VIII at both time points. All images are shown in low and high magnification. After 2 weeks, silk grafts were shown with evidence of neointimal hyperplasia (see 2-week histology of section 2) and a confluent endothelium (see 2-week histology of section 3). After 4 weeks, these changes were less pronounced and tissue remodeling has taken place (see 4-week histology of sections 2 and 3). All scale bars are 200 μm. (Lovett M, Eng G, Kluge J A, Cannizzaro C, Vunjak-Novakovic G, Kaplan D L. Tubular silk scaffolds for small diameter vascular grafts. Organogenesis. 2010; 6:217-24.)

FIGS. 30A-30B are data graphs showing tunable degradation rate of silk tubes by controlling β-sheet crystalline content. In FIG. 30A, FTIR absorbance spectra in the amide I and II region for the tubes: (i) water annealed for 5 hours, (ii) water-annealed for 5 hours followed by 70% MeOH treated for 1 hour, (iii) 70% MeOH treated for 1 hour. The β-sheet contents of those tubes were 34%, 43% and 47%, respectively. Spectra were obtained using a JASCO FT/IR6200 (Easton, Md.). Attenuated Total Reflectance was used for the tubes. All scans were performed with an average of 32 repeats and 4 cm-1 scan resolution. To identify the secondary structures after various treatments, Fourier transform self-deconvolution of the FTIR absorbance spectra in amide I region (1585˜1720 cm-1), was performed using Opus 5.0 software. FIG. 30B shows the results of a degradation assay by protease enzymes. Relationship between the residual mass of various tube formulations vs. time of incubation with Protease XIV solution. The tubes were incubated in protease XIV solution (5 U/mL in PBS, pH 7.4) for interval time periods at 37° C. Enzyme solutions were replaced every two days to maintain enzyme activity. After the specified time, samples were washed with PBS and deionized water. Subsequently, the samples were dried in air for 24 h and further dried in vacuum for 24 h before measuring weight.

FIGS. 31A-31F are hematoxylin and eosin (H&E) staining photographs showing in vivo biodegradation of fabricated silk tubes in mice, e.g., balb/c female mice. (FIGS. 31A-31B) Water annealed for 5 hr; (FIGS. 31C-31D) water-annealed for 5 hours followed by 70% MeOH treated for 1 hour; (FIGS. 31E-31F) 70% MeOH treated for 1 hour. Scale bars represent 200 μm for (FIGS. 31A, 31C, and 31E) and 62.5 μm for (FIGS. 31B, 31D, and 31F), respectively. Tubes were implanted subcutaneously under general anesthesia. After 1 month, the silk biomaterials with surrounded tissues were excised together. After fixation with 4% phosphate-buffered formaldehyde for at least 24 h, the specimens were embedded in paraffin and sectioned into a thickness of 10 μm. The samples underwent routine histological processing with hematoxylin and eosin.

FIG. 32 is a chart showing the mechanical properties of foams that were created using silk solutions and boiling times ranging from 60 minutes to 5 minutes.

### DETAILED DESCRIPTION OF THE INVENTION

While silk fibroin present in native silk exhibits robust mechanical properties, silk fibroin protein can degrade during degumming silk cocoons to remove sericin. While the extraction of the sericin proteins from the fibers is necessary to avoid inflammatory responses in vivo (Panilaitis, B., et, al. Biomaterials, 2003, 24 (18):3079-3085; Altman, G. H. C., et al., 2004, Tissue Regeneration, Inc.: United States, 45), this extraction process results in the degradation of protein chains. Most of the literature on regenerated silk fibroin to date has utilized silk that has been degummed for 20-30 minutes or longer. This degree of degumming results in a broad distribution of silk fibroin weights from undegraded strands of 370 kDa to small fragments of 40-50 kDa and a number average molecular weight on the order of 150 kDa (Yamada, H., et al., Materials Science and Engineering: C, 2001, 14 (1-2):41-46). The impact of this broad molecular weight distribution on the nature of the self-assembly process, and thereby mechanical properties, is incompletely understood. Accordingly, there is a need for improved control in processing silk fibroin from native silk that can preserve robust mechanical properties of natural silk fibroin while carefully controlling for a desired molecular weight distribution. This led to the discovery of new types of silk fibroin-based materials with enhanced mechanical properties and substantially free of sericin.

The inventors have demonstrated inter alia that milder degumming processes (e.g., heating silk cocoons at a temperature of about 90° C. or higher for less than 5 minutes or at a lower temperature (e.g., as low as about 60° C.-70° C.) for a longer period of time (e.g., about 30 minutes or longer) can not only reduce degradation of silk fibroin protein chains and thus generate silk fibroin of higher average molecular weights, but can also substantially remove sericin from native silk fibers. A typical degumming process generally involves heating silk cocoons at a temperature of at least about 90° C. for at least about 20-30 minutes. Accordingly, the inventors have discovered a degumming condition at which surprisingly, a substantial amount of sericin can be removed from native silk fibers to yield a higher molecular weight silk fibroin solution than what is typically achieved. This is the first example of a reconstituted substantially sericin-free silk fibroin solution with a high molecular weight range, which can be subsequently used to form different silk fibroin articles as described herein. Further, the inventors have discovered enhanced mechanical properties of silk fibroin-based materials made from the higher molecular weight silk fibroin. In particular, high molecular weight silk fibroin can be used at a low concentration, for example, as low as 0.5% w/v silk fibroin or lower, to form a mechanically robust silk fibroin-based scaffold with desirable degradation properties. Accordingly, embodiments of various aspects described herein relate to novel compositions comprising a silk-based material of high molecular weight silk fibroin, methods of making the same and uses of the same.

### Compositions Comprising a Solid-State Silk Fibroin or Silk Fibroin Article Having High Molecular Weight (MW) Silk Fibroin

In one aspect, provided herein relates to a composition comprising a solid-state silk fibroin having high molecular weight (MW) silk fibroin. As used herein, the term “high molecular weight (MW) silk fibroin” refers to silk fibroin proteins having an average molecular weight of at least about 100 kDa or more, including, e.g., at least about 150 kDa, at least about 200 kDa, at least about 250 kDa, at least about 300 kDa, at least about 350 kDa or more. In some embodiments, the silk fibroin proteins can have an average molecular weight of at least about 200 kDa or more. In some embodiments, the average molecular weight can be determined from a molecular weight distribution. In these embodiments, the molecular weights of silk fibroin proteins can be described by a molecular weight distribution with an average molecular weight defined herein, for example, of at least about 100 kDa or more, including about 150 kDa, at least about 200 kDa or more. In one embodiment, the molecular weights of silk fibroin proteins can be described by a molecular weight distribution with an average molecular weight of at least about 200 kDa or more. In these embodiments where silk fibroin has a molecular weight distribution, no more than 50%, for example, including, no more than 40%, no more than 30%, no more than 20%, no more than 10%, of the silk fibroin can have a molecular weight of less than 150 kDa, or less than 125 kDa, or less than 100 kDa. In some embodiments, no more than 30% of the silk fibroin can have a molecular weight of less than 100 kDa. Without wishing to be bound by theory, the high molecular weight silk fibroin generally has longer chains.

In other embodiments, all of the silk fibroin proteins can substantially have the same molecular weight as the average molecular weight defined herein (e.g., of at least about 100 kDa, at least about 150 kDa, or at least about 200 kDa or more). The molecular weights of silk fibroin can be generally measured by any methods known in the art, e.g., but not limited to, gel electrophoresis, gel permeation chromatography, light scattering, and/or mass spectrometry.

In some embodiments, the average molecular weight of silk fibroin can refer to the number average molecular weight of silk fibroin, which is the arithmetic mean or average of the molecular weights of individual silk fibroin proteins. Number average molecular weight can be determined by measuring the molecular weight of n silk fibroin proteins, summing the molecular weights of n silk fibroin proteins, and dividing by n. Methods for determining the number average molecular weight of a polymer are known in the art, including, e.g., but not limited to, gel permeation chromatography, and can be used to determine the number average molecular weight of silk fibroin proteins.

In some embodiments, the average molecular weight refers to the weight-average molecular weight of silk fibroin. Weight-average molecular weight (M̅_̅{̅w̅}̅) can be determined as follows:

$$\overline{{M}_{w}} = \frac{{\sum }_{i} {N}_{i} {M}_{i}^{2}}{{\sum }_{i} {N}_{i} {M}_{i}} ,$$

where Nᵢ is the number of silk fibroin proteins with a molecular weight of Mᵢ. Methods for determining the weight-average molecular weight of a polymer are known in the art, including, e.g., but not limited to, static light scattering, small angle neutron scattering, and X-ray scattering, and can be used to determine the weight-average molecular weight of silk fibroin proteins.

In some embodiments, the molecular weights of the silk fibroin defined herein refers to molecular weights of silk fibroin in a solution as measured by gel electrophoresis, e.g., sodium dodecyl sulfate (SDS)-polyacrylamide gel electrophoresis (PAGE). One of skill in the art will readily appreciate that electrophoretic mobility can be influenced by, e.g., protein folding and/or molecular weight. Thus, any difference in protein folding between the marker protein and silk fibroin can also cause a discrepancy in readout of the silk fibroin molecular weight from the actual silk fibroin molecular weight. To account for such measurement discrepancy, for example, one can extract silk dope from silkworm (e.g., B. mori silk worm) and perform a SDS-PAGE analysis. Native fibroin is generally believed to have a molecular weight of about 350-370 kDa (see, e.g., Sasaki and Nodi, Biochimica et Biophysica Acta (BBA)—Protein Structure (1973) 310:76-90). Thus, a shift in the silk fibroin band from about 350-370 kDa on a SDS-PAGE gel can provide an estimate of the discrepancy from the actual molecular weights.

In accordance with some embodiments described herein, high molecular weight silk fibroin can be produced under a milder degumming condition. Accordingly, in some embodiments, high molecular weight silk fibroin can refer to silk fibroin produced by a process comprising degumming silk cocoons at a more gentle condition than a typical degumming condition known in the art. For example, in some embodiments, high molecular weight silk fibroin can refer to silk fibroin produced by a process comprising degumming silk cocoons at a temperature of at least about 90° C. or higher (e.g., up to boiling temperature) for no more than 20 minutes, no more than 15 minutes, no more than 10 minutes, no more than 5 minutes, no more than 4 minutes, no more than 3 minutes, no more than 2 minutes, no more than 1 minute, no more than 30 seconds, or less. In some embodiments, high molecular weight silk fibroin can refer to silk fibroin produced by a process comprising degumming silk cocoons at a temperature of at least about 90° C. for no more than 15 minutes, no more than 10 minutes, no more than 4 minutes, no more than 3 minutes or less.

In alternative embodiments, high molecular weight silk fibroin can refer to silk fibroin produced by a process comprising degumming silk cocoons at a temperature in a range of about 50° C. to about 90°, including, for example, about 60° C. to about 90° C., about 60° C. to less than 90° C., or about 60° C. to about 80° C., for at least about 20 minutes or more, for example, including at least about 30 minutes, at least about 45 minutes, at least about 60 minutes, at least about 90 minutes or more. In some embodiments, high molecular weight silk fibroin can refer to silk fibroin produced by a process comprising degumming silk cocoons at a temperature of about 60° C. to about 90° C. for at least about 30 minutes or longer, including, at least about 45 minutes, at least about 60 minutes or longer. In some embodiments, high molecular weight silk fibroin can refer to silk fibroin produced by a process comprising degumming silk cocoons at a temperature of about 70° C. for at least about 30 minutes or longer, including, at least about 45 minutes, at least about 60 minutes or longer.

Stated another way, in some embodiments, high molecular weight silk fibroin can refer to silk fibroin having a greater average molecular weight than that of silk fibroin after a typical degumming process. For example, high molecular weight silk fibroin can have an average molecular weight of at least about 10% or more, including, e.g., at least about 20%, at least about 30%, at least about 40%, at least about 50%, at least about 60%, at least about 70%, at least about 80%, at least about 90%, at least about 95% or more, greater than the molecular weight of silk fibroin produced by a process comprising degumming silk cocoons at a temperature of at least about 90° C. for about 20-30 minutes. In some embodiments, high molecular weight silk fibroin can have an average molecular weight of at least more than 1 fold, e.g., including, at least about 1.5 fold, at least about 2 fold, at least about 3 fold, at least about 4 fold or more, greater than the molecular weight of silk fibroin produced by a process comprising degumming silk cocoons at a temperature of at least about 90° C. for about 20-30 minutes.

The inventors have surprisingly discovered, in some embodiments, that degumming silk cocoons at a temperature of at least about 90° C. or higher (e.g., up to about boiling temperature) for less than 5 minutes (e.g., 3-5 minutes) is not only desirable to yield silk fibroin (e.g., silk fibroin solution) in high molecular weight ranges, but is also sufficient to substantially remove sericin from the silk fibers to make a high molecular weight silk fibroin solution. Accordingly, in some embodiments, the solid-state silk fibroin of the composition described herein can have high molecular weight silk fibroin and be substantially free of sericin. As used herein, the term “substantially free of sericin” refers to a sericin content of less than 10%, less than 8%, less than 6%, less than 5%, less than 4%, less than 3%, less than 2%, less than 1% or lower. In some embodiments, the term “substantially free of sericin” refers to a sericin content of less than 5% or lower.

Removal of sericin from native silk fibers is desirable due to its implication in inflammatory response in vivo. Accordingly, in some embodiments, the term “substantially free of sericin” can refer to an amount of sericin that does not substantially implicate any inflammatory response in vivo. Examples of an inflammatory response induced by sericin can include, but not limited to, increased production of interleukin (IL)-1 beta and/or tumor necrosis factor (TNF)-alpha by immune cells such as macrophages and monocytes. See, e.g., Aramwit et al., J. Biosci Bioeng. 2009; 107:556-561; Panilaitis B., Biomaterials, 2003. 24:3079-3085; and Altman et al. Immunoneutral Silk-Fiber-Based Medical Devices. 2004; Tissue Regeneration, Inc.: Unites States. p. 45.

High molecular weight silk fibroin can be used at any concentrations in a solid-state silk fibroin or silk fibroin article described herein, depending on desirable material properties in different applications. In some embodiments, high molecular weight silk fibroin can be present in the solid-state silk fibroin or silk fibroin article in an amount of about less than 1 wt % to about 50 wt %, about 0.25 wt % to about 30 wt %, about 0.5 wt % to about 15 wt %, or about 0.5 wt % to about 10 wt %, of the total weight or total volume. In some embodiments, silk fibroin can be present in the solid-state silk fibroin or silk fibroin article in an amount of about less than 1 wt % to about 20 wt % or higher, about 0.25 wt % to about 15 wt %, or about 0.5 wt % to about 10 wt %, of the total weight or volume. In some embodiments, high molecular weight silk fibroin can be present in the solid-state silk fibroin or silk fibroin article in an amount of about 5 wt % to about 50 wt %, about 10 wt % to about 40 wt %, about 20 wt % to about 30 wt %, of the total weight or volume.

Low Concentration of Silk Fibroin:

In some embodiments, high molecular weight silk fibroin can be used at a low concentration (e.g., in a range of about 5% w/v to as low as 0.5% w/v silk fibroin solution) to form a mechanically stable (e.g., ability to maintain shape and/or volume) but fast-degrading solid-state silk fibroin article or silk fibroin scaffold. As used herein, the term “fast-degrading” refers to an ability of a silk-based material to degrade at least about 10% or more, including, e.g., at least about 20%, at least about 30%, at least about 40% or more, of silk fibroin over a period of about 1 week in vivo or in the presence of a protease or silk-degrading enzyme.

As used herein, the term “mechanically stable” refers to an ability of a silk-based material to maintain shape and/or volume after physical manipulation, e.g., during silk processing, handling, and/or application (e.g., implantation). The term “maintain shape and/or volume” refers to no substantial change in shape and/or volume of a silk fibroin-based material, or alternatively, the change in shape and/or volume of a silk fibroin-based material being less than 30% or lower (including, e.g., less than 20%, less than 10% or lower), after physical manipulation, e.g., during silk processing, handling, and/or application (e.g., implantation). In some embodiments, a mechanically-stable silk fibroin-based material can deform under loading but restore to its original shape and/or shape (e.g., restore to at least about 50% or more, including, for example, at least about 60%, at least about 70%, at least about 80%, at least about 90%, at least about 95% or more, of its original shape and/or shape) after release of the loading.

Accordingly, another aspect provided herein relates to a composition comprising a mechanically-stable solid-state silk fibroin or silk fibroin article comprising a low concentration of silk fibroin. In some embodiments, the mechanically-stable solid-state silk fibroin or silk fibroin article can comprise a low concentration of high molecular weight silk fibroin. As used herein, the term “low concentration of silk fibroin” can refer to a mass concentration of silk fibroin (e.g., high molecular weight silk fibroin) present in a solid-state silk fibroin or silk fibroin article, at or below which high molecular weight silk fibroin, but not relatively low molecular weight silk fibroin (e.g., silk fibroin produced by a process involving a typical degumming process—heating silk cocoons at a temperature of at least about 90° C. for about 20-30 minutes), can form a mechanically-stable structure. In some embodiments, the term “low concentration of silk fibroin” can refer to a mass concentration of silk fibroin (e.g., high molecular weight silk fibroin) present in a solid-state silk fibroin or silk fibroin article, at or below which the resulting mechanically-stable structure can degrade in vivo, or in the presence of a protease or silk-degrading enzyme, at a rate at least comparable to or faster than the degradation rate of a solid-state silk fibroin or silk fibroin article formed from relatively low molecular weight silk fibroin at a minimum concentration required to yield a mechanically-stable structure. In some embodiments, the term “low concentration of silk fibroin” can refer to a mass concentration of silk fibroin (e.g., high molecular weight silk fibroin) present in a solid-state silk fibroin or silk fibroin article that is no more than 2% (w/v or w/w), including, e.g., no more than 1% (w/v or w/w), or no more than 0.5% (w/v or w/w), of the volume or mass of the solid-state silk fibroin or silk fibroin article.

In some embodiments, the volume of the resulting solid-state silk fibroin or silk fibroin article can be substantially the same as the volume of the silk fibroin solution used to form the solid-state silk fibroin or silk fibroin article. For example, there is no shrinkage in volume during formation of the solid-state silk fibroin or silk fibroin article from a specific volume of the silk fibroin solution. In these embodiments, the mass concentration of silk fibroin present in a solid-state silk fibroin or silk fibroin article can be substantially the same as the mass concentration of silk fibroin in a solution used to form the solid-state silk fibroin or silk fibroin article.

In other embodiments, the volume of the resulting solid-state silk fibroin or silk fibroin article can be smaller or larger than the volume of the silk fibroin solution used to form the solid-state silk fibroin or silk fibroin article. For example, there is a reduction or expansion in volume during formation of the solid-state silk fibroin or silk fibroin article from a specific volume of the silk fibroin solution.

The mechanical stability of the solid-state silk fibroin or silk fibroin article having a low concentration of silk fibroin described herein can be characterized by at least one of the mechanical properties, including, e.g., elastic modulus, shear modulus, tensile strength, compressive strength, and/or stiffness. For example, in some embodiments, the solid-state silk fibroin or silk fibroin article having a low concentration of silk fibroin (e.g., high molecular weight silk fibroin) can have an elastic modulus of at least about 0.1 kPa or more, including, e.g., at least about 0.2 kPa, at least about 0.3 kPa, at least about 0.4 kPa, at least about 0.5 kPa, at least about 0.6 kPa, at least about 0.7 kPa, at least about 0.8 kPa, at least about 0.9 kPa, at least about 1 kPa, at least about 2 kPa, at least about 3 kPa, at least about 4 kPa or higher. In some embodiments, the solid-state silk fibroin or silk fibroin article having a low concentration of silk fibroin (e.g., high molecular weight silk fibroin) can have an elastic modulus of at least about 0.2 kPa, or at least about 0.7 kPa, or more.

In other embodiments, the solid-state silk fibroin or silk fibroin article having a low concentration of silk fibroin (e.g., high molecular weight silk fibroin) can have an ultimate tensile strength of at least about 3 kPa or more, including, e.g., at least about 5 kPa, at least about 7.5 kPa, at least about 10 kPa, at least about 12.5 kPa, at least about 15 kPa, at least about 17.5 kPa, at least about 20 kPa, at least about 25 kPa or higher. In some embodiments, the solid-state silk fibroin or silk fibroin article having a low concentration of silk fibroin (e.g., high molecular weight silk fibroin) can have an ultimate tensile strength of at least about 5 kPa or at least about 10 kPa, or at least about 20 kPa, or more.

High Concentration of Silk Fibroin:

As described above, high molecular weight silk fibroin can be used at low concentrations. Alternatively, higher concentrations of high molecular weight silk fibroin can be desirable for use in other applications. As used herein, the term “higher concentrations of silk fibroin” can refer to concentrations of silk fibroin (e.g., high molecular weight silk fibroin) that are higher than the low concentrations as defined herein. In some embodiments, the term “higher concentrations of silk fibroin” can refer to a mass concentration of silk fibroin (e.g., high molecular weight silk fibroin) present in a solid-state silk fibroin or silk fibroin article that is more than 1% (w/v or w/w), including, e.g., more than 2% (w/v or w/w), or more than 3% (w/v or w/w), or more than 4% (w/v or w/w), or more than 5% (w/v or w/w), or more than 6% (w/v or w/w), or more than 7% (w/v or w/w), or more than 8% (w/v or w/w), or more than 9% (w/v or w/w), of the volume or mass of the solid-state silk fibroin or silk fibroin article. For example, higher concentrations of high molecular weight silk fibroin can be used to yield a solid-state silk fibroin or silk fibroin article with enhanced mechanical properties and/or slower degradation rate. In these embodiments, the solid-state silk fibroin or silk fibroin article having a higher concentration of silk fibroin (e.g., high molecular weight silk fibroin) can have an elastic modulus of at least about 0.7 kPa or more, including, e.g., at least about 0.8 kPa, at least about 0.9 kPa, at least about 1 kPa, at least about 1.5 kPa, at least about 2 kPa, at least about 3 kPa, at least about 4 kPa, at least about 5 kPa, at least about 6 kPa, or higher. In some embodiments, the solid-state silk fibroin or silk fibroin article having a higher concentration of silk fibroin (e.g., high molecular weight silk fibroin) can have an elastic modulus of at least about 1 kPa, or at least about 2 kPa, or more.

In other embodiments, the solid-state silk fibroin or silk fibroin article having a higher concentration of silk fibroin (e.g., high molecular weight silk fibroin) can have an ultimate tensile strength of at least about 20 kPa or more, including, e.g., at least about 30 kPa, at least about 40 kPa, at least about 50 kPa, at least about 60 kPa, at least about 70 kPa, at least about 80 kPa, at least about 90 kPa, at least about 100 kPa, at least about 200 kPa or higher. In some embodiments, the solid-state silk fibroin or silk fibroin article having a higher concentration of silk fibroin (e.g., high molecular weight silk fibroin) can have an ultimate tensile strength of at least about 20 kPa or at least about 40 kPa, or at least about 80 kPa, or more.

High molecular weight silk fibroin can be used to form a solid-state silk fibroin or silk fibroin article in any form. For example, the solid-state silk fibroin or silk fibroin article can be present in a form selected from the group consisting of a film (See, e.g., U.S. Pat. Nos. 7,674,882; and 8,071,722); a sheet (see, e.g., PCT/US13/24744 filed Feb. 5, 2013); a gel (see, e.g., U.S. Pat. No. 8,187,616; and U.S. Pat. App. Nos. US 2012/0070427; and US 2011/0171239); a mesh or a mat (see, e.g., International Pat. App. No. WO 2011/008842); a non-woven mat or fabric (see, e.g., International Pat. App. Nos. WO 2003/043486 and WO 2004/080346); a scaffold (see, e.g., U.S. Pat. Nos. 7,842,780; and 8,361,617); a tube (see, e.g., U.S. Pat. App. No. US 2012/0123519; International Pat. App. No. WO 2009/126689; and International Pat. App. Serial No. PCT/US13/30206 filed Mar. 11, 2013); a slab or block; a fiber (see, e.g., U.S. Pat. App. No. US 2012/0244143); a 3 dimensional construct (see, e.g., International Pat. App. No. WO 2012/145594, including, but not limited to, an implant, a screw, a plate); a high-density material (see, e.g., International Pat. App. Serial No. PCT/US13/35389 filed Apr. 5, 2013); a porous material such as a foam or sponge (see, e.g., U.S. Pat. Nos. 7,842,780; and 8,361,617); a coating (see, e.g., International Patent Application Nos. WO 2007/016524; WO 2012/145652); a magnetic-responsive material (see, e.g., International Pat. App. Serial No. PCT/US13/36539 filed Apr. 15, 2013); a needle (see, e.g., International Patent Application No. WO 2012/054582); a machinable material (see, e.g., U.S. Prov. App. No. 61/808,768 filed Apr. 5, 2013); powder; a lyophilized material; or any combinations thereof. The contents of each of the aforementioned patent applications are incorporated herein by reference in their entireties.

Silk Fibroin:

Silk fibroin is a particularly appealing protein polymer candidate to be used for various embodiments described herein, e.g., because of its versatile processing e.g., all-aqueous processing (Sofia et al., 54 J. Biomed. Mater. Res. 139 (2001); Perry et al., 20 Adv. Mater. 3070-72 (2008)), relatively easy functionalization (Murphy et al., 29 Biomat. 2829-38 (2008)), and biocompatibility (Santin et al., 46 J. Biomed. Mater. Res. 382-9 (1999)). For example, silk has been approved by U.S. Food and Drug Administration as a tissue engineering scaffold in human implants. See Altman et al., 24 Biomaterials: 401 (2003).

As used herein, the term “silk fibroin” or “fibroin” includes silkworm fibroin and insect or spider silk protein. See e.g., Lucas et al., 13 Adv. Protein Chem. 107 (1958). Any type of silk fibroin can be used according to aspects of the present invention. Silk fibroin produced by silkworms, such as Bombyx mori, is the most common and represents an earth-friendly, renewable resource. For instance, silk fibroin can be attained by extracting sericin from the cocoons of B. mori. Organic silkworm cocoons are also commercially available. There are many different silks, however, including spider silk (e.g., obtained from Nephila clavipes), transgenic silks, genetically engineered silks (recombinant silk), such as silks from bacteria, yeast, mammalian cells, transgenic animals, or transgenic plants, and variants thereof, that can be used. See for example, WO 97/08315 and U.S. Pat. No. 5,245,012, content of both of which is incorporated herein by reference in its entirety. In some embodiments, silk fibroin can be derived from other sources such as spiders, other silkworms, bees, and bioengineered variants thereof. In some embodiments, silk fibroin can be extracted from a gland of silkworm or transgenic silkworms. See for example, WO2007/098951, content of which is incorporated herein by reference in its entirety. In some embodiments, silk fibroin is free, or essentially free of sericin, i.e., silk fibroin is a substantially sericin-depleted silk fibroin.

In some embodiments, the high molecular weight silk fibroin can include an amphiphilic peptide. In other embodiments, the silk fibroin can exclude an amphiphilic peptide. “Amphiphilic peptides” possess both hydrophilic and hydrophobic properties. Amphiphilic molecules can generally interact with biological membranes by insertion of the hydrophobic part into the lipid membrane, while exposing the hydrophilic part to the aqueous environment. In some embodiment, the amphiphilic peptide can comprise a RGD motif. An example of an amphiphilic peptide is a 23RGD peptide having an amino acid sequence: HOOC-Gly-ArgGly-Asp-Ile-Pro-Ala-Ser-Ser-Lys-Gly-Gly-Gly-Gly-SerArg-Leu-Leu-Leu-Leu-Leu-Leu-Arg-NH2. Other examples of amphiphilic peptides include the ones disclosed in the U.S. Patent App. No.: US 2011/0008406, the content of which is incorporated herein by reference.

In various embodiments, the high molecular weight silk fibroin can be modified for different applications and/or desired mechanical or chemical properties (e.g., to facilitate formation of a gradient of an additive (e.g., an active agent) in silk fibroin-based materials). One of skill in the art can select appropriate methods to modify silk fibroins, e.g., depending on the side groups of the silk fibroins, desired reactivity of the silk fibroin and/or desired charge density on the silk fibroin. In one embodiment, modification of silk fibroin can use the amino acid side chain chemistry, such as chemical modifications through covalent bonding, or modifications through charge-charge interaction. Exemplary chemical modification methods include, but are not limited to, carbodiimide coupling reaction (see, e.g. U.S. Patent Application. No. US 2007/0212730), diazonium coupling reaction (see, e.g., U.S. Patent Application No. US 2009/0232963), avidin-biotin interaction (see, e.g., International Application No.: WO 2011/011347) and pegylation with a chemically active or activated derivatives of the PEG polymer (see, e.g., International Application No. WO 2010/057142). Silk fibroin can also be modified through gene modification to alter functionalities of the silk protein (see, e.g., International Application No. WO 2011/006133). For instance, the silk fibroin can be genetically modified, which can provide for further modification of the silk such as the inclusion of a fusion polypeptide comprising a fibrous protein domain and a mineralization domain, which can be used to form an organic-inorganic composite. See WO 2006/076711. In some embodiments, the silk fibroin can be genetically modified to be fused with a protein, e.g., a therapeutic protein. Additionally, the silk fibroin-based material can be combined with a chemical, such as glycerol, that, e.g., affects flexibility of the material. See, e.g., WO 2010/042798, Modified Silk films Containing Glycerol. The contents of the aforementioned patent applications are all incorporated herein by reference.

Active Agents:

In some embodiments, a solid-state silk fibroin or silk fibroin article can comprise at least one active agent as described in the section “Exemplary active agents” below. The active agent can be dispersed homogeneously or heterogeneously within silk fibroin, or dispersed in a gradient, e.g., using the carbodiimide-mediated modification method described in the U.S. Patent Application No. US 2007/0212730. In some embodiments, the active agent can be coated on a surface of the solid-state silk fibroin or silk fibroin article, e.g., via diazonium coupling reaction (see, e.g., U.S. Patent Application No. US 2009/0232963), and/or avidin-biotin interaction (see, e.g., International Application No.: WO 2011/011347). Non-limiting examples of the active agent can include cells, proteins, peptides, nucleic acids, nucleic acid analogs, nucleotides or oligonucleotides, peptide nucleic acids, aptamers, antibodies or fragments or portions thereof, antigens or epitopes, hormones, hormone antagonists, growth factors or recombinant growth factors and fragments and variants thereof, cell attachment mediators, cytokines, enzymes, antibiotics or antimicrobial compounds, viruses, toxins, therapeutic agents and prodrugs thereof, small molecules, and any combinations thereof. See, e.g., the International Patent Application No. WO/2012/145739 for compositions and methods for stabilization of at least one active agent with silk fibroin. In some embodiments, at least one active agent can be genetically fused to silk fibroin to form a fusion protein. The contents of the aforementioned patent applications are incorporated herein by reference.

Any amounts of an active agent can be present in a solid-state silk fibroin or silk fibroin article. For example, in some embodiments, an active agent can be present in the solid-state silk fibroin or silk fibroin article at a concentration of about 0.001 wt % to about 50 wt %, about 0.005 wt % to about 40 wt %, about 0.01 wt % to about 30 wt %, about 0.05 wt % to about 20 wt %, about 0.1 wt % to about 10 wt %, or about 0.5 wt % to about 5 wt %.

Additives:

In some embodiments, the composition described herein can comprise one or more (e.g., one, two, three, four, five or more) additives. In some embodiments, the additive(s) can be incorporated into the solid-state silk fibroin or silk fibroin article. Without wishing to be bound by theory, an additive can provide one or more desirable properties to the composition or solid-state silk fibroin or silk fibroin article, e.g., strength, flexibility, ease of processing and handling, biocompatibility, bioresorbility, lack of air bubbles, surface morphology, and the like. The additive can be covalently or non-covalently linked with silk fibroin and/or can be integrated homogenously or heterogeneously within the silk fibroin-based material.

An additive can be selected from small organic or inorganic molecules; biocompatible polymers; plasticizers; small organic or inorganic molecules; saccharides; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; glycogens or other sugars; immunogens; antigens; an extract made from biological materials such as bacteria, plants, fungi, or animal cells; animal tissues; naturally occurring or synthetic compositions; and any combinations thereof. Furthermore, the additive can be in any physical form. For example, the additive can be in the form of a particle, a fiber, a film, a tube, a gel, a mesh, a mat, a non-woven mat, a powder, a liquid, or any combinations thereof. In some embodiments, the additive can be a particle (e.g., a microparticle or nanoparticle).

Total amount of additives in the composition or in the solid-state silk fibroin can be in a range of about 0.1 wt % to about 0.99 wt %, about 0.1 wt % to about 70 wt %, about 5 wt % to about 60 wt %, about 10 wt % to about 50 wt %, about 15 wt % to about 45 wt %, or about 20 wt % to about 40 wt %, of the total silk fibroin in the composition.

In some embodiments, the additive can include a calcium phosphate (CaP) material. As used herein, the term “calcium phosphate material” refers to any material composed of calcium and phosphate ions. The term “calcium phosphate material” is intended to include naturally occurring and synthetic materials composed of calcium and phosphate ions. The ratio of calcium to phosphate ions in the calcium phosphate materials is preferably selected such that the resulting material is able to perform its intended function. For convenience, the calcium to phosphate ion ratio is abbreviated as the “Ca/P ratio.” In some embodiments, the Ca/P ratio can range from about 1:1 to about 1.67 to 1. In some embodiments, the calcium phosphate material can be calcium deficient. By “calcium deficient” is meant a calcium phosphate material with a calcium to phosphate ratio of less than about 1.6 as compared to the ideal stoichiometric value of approximately 1.67 for hydroxyapatite

The calcium phosphate material can be in the form of particles. Without limitations, the calcium phosphate material particles can be of any desired size. In some embodiments, the calcium phosphate material particles can have a size ranging from about 0.01 μm to about 1000 μm, about 0.05 μm to about 500 μm, about 0.1 μm to about 250 μm, about 0.25 μm to about 200 μm, or about 0.5 μm to about 100 μm. Further, the calcium phosphate material particle can be of any shape or form, e.g., spherical, rod, elliptical, cylindrical, capsule, or disc.

In some embodiments, the calcium phosphate material particle can be a microparticle or a nanoparticle. In some embodiments, the calcium phosphate material particle can have a particle size of about 0.01 μm to about 1000 μm, about 0.05 μm to about 750 μm, about 0.1 μm to about 500 μm, about 0.25 μm to about 250 μm, or about 0.5 μm to about 100 μm. In some embodiments, the silk particle can have a particle size of about 0.1 nm to about 1000 nm, about 0.5 nm to about 500 nm, about 1 nm to about 250 nm, about 10 nm to about 150 nm, or about 15 nm to about 100 nm.

The calcium phosphate material can be selected, for example, from one or more of brushite, octacalcium phosphate, tricalcium phosphate (also referred to as tricalcic phosphate and calcium orthophosphate), calcium hydrogen phosphate, calcium dihydrogen phosphate, apatite, and/or hydroxyapatite. Further, tricalcium phosphate (TCP) can be in the alpha or the beta crystal form. In some embodiments, the calcium phosphate material is beta-tricalcium phosphate or apatite, e.g., hydroxyapatite (HA).

The amount of the calcium phosphate material in the composition or solid-state silk fibroin can range from about 1% to about 99% (w/w or w/v). In some embodiments, the amount of the calcium phosphate material in the composition or solid-state silk fibroin can be from about 5% to about 95% (w/w or w/v), from about 10% to about 90% (w/w or w/v), from about 15% to about 80% (w/w or w/v), from about 20% to about 75% (w/w or w/v), from about 25% to about 60% (w/w or w/v), or from about 30% to about 50% (w/w or w/v). In some embodiments, the amount of the calcium phosphate material in the composition or solid-state silk fibroin can be less than 20%.

Generally, the composition can comprise any ratio of high molecular weight silk fibroin to calcium phosphate material. For example, the ratio of silk fibroin to calcium phosphate material in the composition can range from about 1000:1 to about 1:1000. The ratio can be based on weight or moles. In some embodiments, the ratio of silk fibroin to calcium phosphate material in the solution can range from about 500:1 to about 1:500 (w/w), from about 250:1 to about 1:250 (w/w), from about 50:1 to about 1:200 (w/w), from about 10:1 to about 1:150 (w/w) or from about 5:1 to about 1:100 (w/w). In some embodiments, ratio of silk fibroin to calcium phosphate material in the composition can be about 1:99 (w/w), about 1:4 (w/w), about 2:3 (w/w), about 1:1 (w/w) or about 4:1 (w/w).

In some embodiments, the composition and/or solid-state silk fibroin can comprise magnetic particles to form magneto-sensitive silk fibroin-based materials as described in International Patent Application No. PCT/US13/36539 filed Apr. 15, 2013, the content of which is incorporated herein by reference.

In some embodiments, the composition or the solid-state silk fibroin can comprise a silk material as an additive, for example, to produce a silk fibroin composite (e.g., 100% silk composite) with improved mechanical properties. Examples of silk materials that can be used as an additive include, without limitations, silk particles, silk fibers, silk micron-sized fibers, silk powder and unprocessed silk fibers. In some embodiments, the additive can be a silk particle or powder. Various methods of producing silk fibroin particles (e.g., nanoparticles and microparticles) are known in the art. In some embodiments, the silk particles can be produced by a polyvinyl alcohol (PVA) phase separation method as described in, e.g., International App. No. WO 2011/041395, the content of which is incorporated herein by reference in its entirety. Other methods for producing silk fibroin particles are described, for example, in U.S. App. Pub. No. U.S. 2010/0028451 and PCT App. Pub. No.: WO 2008/118133 (using lipid as a template for making silk microspheres or nanospheres), and in Wenk et al. J Control Release, Silk fibroin spheres as a platform for controlled drug delivery, 2008; 132: 26-34 (using spraying method to produce silk microspheres or nanospheres), content of all of which is incorporated herein by reference in its entirety.

Generally, silk fibroin particles or powder can be obtained by inducing gelation in a silk fibroin solution and reducing the resulting silk fibroin gel into particles, e.g., by grinding, cutting, crushing, sieving, sifting, and/or filtering. Silk fibroin gels can be produced by sonicating a silk fibroin solution; applying a shear stress to the silk solution; modulating the salt content of the silk solution; and/or modulating the pH of the silk solution. The pH of the silk fibroin solution can be altered by subjecting the silk solution to an electric field and/or reducing the pH of the silk solution with an acid. Methods for producing silk gels using sonication are described for example in U.S. Pat. App. Pub No. U.S. 2010/0178304 and Int. Pat. App. Pub. No. WO 2008/150861, contents of both which are incorporated herein by reference in their entirety. Methods for producing silk fibroin gels using shear stress are described, for example, in International Patent App. Pub. No.: WO 2011/005381, the content of which is incorporated herein by reference in its entirety. Methods for producing silk fibroin gels by modulating the pH of the silk solution are described, for example, in U.S. Pat. App. Pub. No.: US 2011/0171239, the content of which is incorporated herein by reference in its entirety.

In some embodiments, silk particles can be produced using a freeze-drying method as described in U.S. Provisional Application Ser. No. 61/719,146, filed Oct. 26, 2012; and International Pat. App. No. PCT/US13/36356 filed: Apr. 12, 2013, content of each of which is incorporated herein by reference in its entirety. Specifically, a silk fibroin foam can be produced by freeze-drying a silk solution. The foam then can be reduced to particles. For example, a silk solution can be cooled to a temperature at which the liquid carrier transforms into a plurality of solid crystals or particles and removing at least some of the plurality of solid crystals or particles to leave a porous silk material (e.g., silk foam). After cooling, liquid carrier can be removed, at least partially, by sublimation, evaporation, and/or lyophilization. In some embodiments, the liquid carrier can be removed under reduced pressure.

Optionally, the conformation of the silk fibroin in the silk fibroin foam can be altered after formation. Without wishing to be bound by theory, the induced conformational change can alter the crystallinity of the silk fibroin in the silk particles, e.g., silk II beta-sheet crystallinity. This can alter the rate of release of an active agent from the silk matrix. The conformational change can be induced by any methods known in the art, including, but not limited to, alcohol immersion (e.g., ethanol, methanol), water annealing, water vapor annealing, heat annealing, shear stress (e.g., by vortexing), ultrasound (e.g., by sonication), pH reduction (e.g., pH titration), and/or exposing the silk particles to an electric field and any combinations thereof.

In some embodiments, no conformational change in the silk fibroin is induced, i.e., crystallinity of the silk fibroin in the silk fibroin foam is not altered or changed before subjecting the foam to particle formation.

After formation, the silk fibroin foam can be subjected to grinding, cutting, crushing, or any combinations thereof to form silk particles. For example, the silk fibroin foam can be blended in a conventional blender or milled in a ball mill to form silk particles of desired size.

Without limitations, the silk fibroin particles can be of any desired size. In some embodiments, the particles can have a size ranging from about 0.01 μm to about 1000 μm, about 0.05 μm to about 500 μm, about 0.1 μm to about 250 μm, about 0.25 μm to about 200 μm, or about 0.5 μm to about 100 μm. Further, the silk particle can be of any shape or form, e.g., spherical, rod, elliptical, cylindrical, capsule, or disc.

In some embodiments, the silk fibroin particle can be a microparticle or a nanoparticle. In some embodiments, the silk particle can have a particle size of about 0.01 μm to about 1000 μm, about 0.05 μm to about 750 μm, about 0.1 μm to about 500 μm, about 0.25 μm to about 250 μm, or about 0.5 μm to about 100 μm. In some embodiments, the silk particle has a particle size of about 0.1 nm to about 1000 nm, about 0.5 nm to about 500 nm, about 1 nm to about 250 nm, about 10 nm to about 150 nm, or about 15 nm to about 100 nm.

The amount of the silk fibroin particles in the composition or solid-state silk fibroin can range from about 1% to about 99% (w/w or w/v). In some embodiments, the amount the silk particles in the composition or solid-state silk fibroin can be from about 5% to about 95% (w/w or w/v), from about 10% to about 90% (w/w or w/v), from about 15% to about 80% (w/w or w/v), from about 20% to about 75% (w/w or w/v), from about 25% to about 60% (w/w or w/v), or from about 30% to about 50% (w/w or w/v).). In some embodiments, the amount of the silk particles in the composition or solid-state silk fibroin can be less than 20%.

Generally, the composition described herein can comprise any ratio of high molecular weight silk fibroin to silk fibroin particles. For example, the ratio of silk fibroin to silk particles in the solution can range from about 1000:1 to about 1:1000. The ratio can be based on weight or moles. In some embodiments, the ratio of high molecular weight silk fibroin to silk particles in the solution can range from about 500:1 to about 1:500 (w/w), from about 250:1 to about 1:250 (w/w), from about 50:1 to about 1:200 (w/w), from about 10:1 to about 1:150 (w/w) or from about 5:1 to about 1:100 (w/w). In some embodiments, ratio of high molecular weight silk fibroin to silk particles in the solution can be about 1:99 (w/w), about 1:4 (w/w), about 2:3 (w/w), about 1:1 (w/w) or about 4:1 (w/w). In some embodiments, the amount of silk particles is equal to or less than the amount of the silk fibroin, i.e., a silk fibroin to silk particle ratio of 1:≤1. In some embodiments, the ratio of high molecular weight silk fibroin to silk particles in the composition can be about 1:1, about 1:0.75, about 1:0.5, or about 1:0.25.

In some embodiments, the additive can be a silk fiber. In some embodiments, silk fibers can be chemically attached by redissolving part of the fiber in HFIP and attaching to the composition or solid-state silk fibroin, for example, as described in US patent application publication no. US20110046686, the content of which is incorporated herein by reference.

In some embodiments, the silk fibers can be microfibers or nanofibers. In some embodiments, the additive can be micron-sized silk fiber (10-600 μm). Micron-sized silk fibers can be obtained by hydrolyzing the degummed silk fibroin or by increasing the boing time of the degumming process. Alkali hydrolysis of silk fibroin to obtain micron-sized silk fibers is described for example in Mandal et al., PNAS, 2012, doi: 10.1073/pnas.1119474109; and PCT application no. PCT/US13/35389, filed Apr. 5, 2013, content of all of which is incorporated herein by reference. Because regenerated silk fibers made from HFIP silk solutions are mechanically strong, in some embodiments, the regenerated silk fibers can also be used as an additive.

In some embodiments, the silk fiber can be an unprocessed silk fiber, e.g., raw silk or raw silk fiber. The term “raw silk” or “raw silk fiber” refers to silk fiber that has not been treated to remove sericin, and thus encompasses, for example, silk fibers taken directly from a cocoon. Thus, by unprocessed silk fiber is meant silk fibroin, obtained directly from the silk gland. When silk fibroin, obtained directly from the silk gland, is allowed to dry, the structure is referred to as silk I in the solid state. Thus, an unprocessed silk fiber comprises silk fibroin mostly in the silk I conformation. A regenerated or processed silk fiber on the other hand comprises silk fibroin having a substantial silk II or beta-sheet crystallinity.

In some embodiments, the additive can comprise at least one biocompatible polymer, including at least two biocompatible polymers, at least three biocompatible polymers or more. For example, the composition and/or the solid-state silk fibroin can comprise one or more biocompatible polymers in a total concentration of about 0.1 wt % to about 70 wt %, about 1 wt % to about 60 wt %, about 10 wt % to about 50 wt %, about 15 wt % to about 45 wt % or about 20 wt % to about 40 wt %. In some embodiments, the biocompatible polymer(s) can be incorporated homogenously or heterogeneously into the solid-state silk fibroin or silk fibroin article. In other embodiments, the biocompatible polymer(s) can be coated on a surface of the solid-state silk fibroin or silk fibroin article. In any embodiments, the biocompatible polymer(s) can be covalently or non-covalently linked to silk fibroin in a solid-state silk fibroin or silk fibroin article. In some embodiments, the biocompatible polymer(s) can be blended with silk fibroin within a solid-state silk fibroin or silk fibroin article. Examples of the biocompatible polymers can include non-degradable and/or biodegradable polymers, e.g., but are not limited to, polyethylene oxide (PEO), polyethylene glycol (PEG), collagen, fibronectin, keratin, polyaspartic acid, polylysine, alginate, chitosan, chitin, hyaluronic acid, pectin, polycaprolactone, polylactic acid, polyglycolic acid, polyhydroxyalkanoates, dextrans, polyanhydrides, polymer, PLA-PGA, polyanhydride, polyorthoester, polycaprolactone, polyfumarate, collagen, chitosan, alginate, hyaluronic acid, other biocompatible and/or biodegradable polymers and any combinations thereof. See, e.g., International Application Nos.: WO 04/062697; WO 05/012606. The contents of the international patent applications are all incorporated herein by reference. Other exemplary biocompatible polymers amenable to use according to the present disclosure include those described for example in U.S. Pat. Nos. 6,302,848; 6,395,734; 6,127,143; 5,263,992; 6,379,690; 5,015,476; 4,806,355; 6,372,244; 6,310,188; 5,093,489; 387,413; 6,325,810; 6,337,198; 6,267,776; 5,576,881; 6,245,537; 5,902,800; and 5,270,419, content of all of which is incorporated herein by reference.

In some embodiments, the biocompatible polymer can comprise PEG or PEO. As used herein, the term “polyethylene glycol” or “PEG” means an ethylene glycol polymer that contains about 20 to about 2000000 linked monomers, typically about 50-1000 linked monomers, usually about 100-300. PEG is also known as polyethylene oxide (PEO) or polyoxyethylene (POE), depending on its molecular weight. Generally PEG, PEO, and POE are chemically synonymous, but PEG has previously tended to refer to oligomers and polymers with a molecular mass below 20,000 g/mol, PEO to polymers with a molecular mass above 20,000 g/mol, and POE to a polymer of any molecular mass. PEG and PEO are liquids or low-melting solids, depending on their molecular weights. PEGs are prepared by polymerization of ethylene oxide and are commercially available over a wide range of molecular weights from 300 g/mol to 10,000,000 g/mol. While PEG and PEO with different molecular weights find use in different applications, and have different physical properties (e.g. viscosity) due to chain length effects, their chemical properties are nearly identical. Different forms of PEG are also available, depending on the initiator used for the polymerization process—the most common initiator is a monofunctional methyl ether PEG, or methoxypoly(ethylene glycol), abbreviated mPEG. Lower-molecular-weight PEGs are also available as purer oligomers, referred to as monodisperse, uniform, or discrete PEGs are also available with different geometries.

As used herein, the term PEG is intended to be inclusive and not exclusive. The term PEG includes poly(ethylene glycol) in any of its forms, including alkoxy PEG, difunctional PEG, multiarmed PEG, forked PEG, branched PEG, pendent PEG (i.e., PEG or related polymers having one or more functional groups pendent to the polymer backbone), or PEG With degradable linkages therein. Further, the PEG backbone can be linear or branched. Branched polymer backbones are generally known in the art. Typically, a branched polymer has a central branch core moiety and a plurality of linear polymer chains linked to the central branch core. PEG is commonly used in branched forms that can be prepared by addition of ethylene oxide to various polyols, such as glycerol, pentaerythritol and sorbitol. The central branch moiety can also be derived from several amino acids, such as lysine. The branched poly(ethylene glycol) can be represented in general form as R(-PEG-OH)m in which R represents the core moiety, such as glycerol or pentaerythritol, and m represents the number of arms. Multi-armed PEG molecules, such as those described in U.S. Pat. No. 5,932,462, which is incorporated by reference herein in its entirety, can also be used as biocompatible polymers.

Some exemplary PEGs include, but are not limited to, PEG20, PEG30, PEG40, PEG60, PEG80, PEG100, PEG115, PEG200, PEG 300, PEG400, PEG500, PEG600, PEG1000, PEG1500, PEG2000, PEG3350, PEG4000, PEG4600, PEG5000, PEG6000, PEG8000, PEG11000, PEG12000, PEG15000, PEG 20000, PEG250000, PEG500000, PEG100000, PEG2000000 and the like. In some embodiments, PEG is of MW 10,000 Dalton. In some embodiments, PEG is of MW 100,000, i.e. PEO of MW 100,000.

In some embodiments, the additive can include an enzyme that hydrolyzes silk fibroin. Without wishing to be bound by theory, such enzymes can be used to control the degradation of the composition and/or solid-state silk fibroin.

In some embodiments, the solid-state silk fibroin can have a porosity of at least about 1%, at least about 5%, at least about 10%, at least about 20%, at least about 30%, at least about 40%, at least about 50%, at least about 60%, at least about 70%, at least about 80%, at least about 90%, or higher. As used herein, the term “porosity” is a measure of void spaces in a material and is a fraction of volume of voids over the total volume, as a percentage between 0 and 100% (or between 0 and 1). Determination of porosity is well known to a skilled artisan, e.g., using standardized techniques, such as mercury porosimetry and gas adsorption, e.g., nitrogen adsorption.

The porous solid-state silk fibroin can have any pore size. As used herein, the term “pore size” refers to a diameter or an effective diameter of the cross-sections of the pores. The term “pore size” can also refer to an average diameter or an average effective diameter of the cross-sections of the pores, based on the measurements of a plurality of pores. The effective diameter of a cross-section that is not circular equals the diameter of a circular cross-section that has the same cross-sectional area as that of the non-circular cross-section. In some embodiments, the pores of the solid-state silk fibroin can have a size distribution ranging from about 50 nm to about 1000 μm, from about 250 nm to about 500 μm, from about 500 nm to about 250 μm, from about 1 μm to about 200 μm, from about 10 μm to about 150 μm, or from about 50 μm to about 100 μm. In some embodiments, the solid-state silk fibroin can be swellable when hydrated. The sizes of the pores can then change depending on the water content in the silk matrix. In some embodiment, the pores can be filled with a fluid such as water or air.

Another aspect provided herein relates to articles of manufacture comprising one or more embodiments of the composition described herein. Examples of articles of manufacture can include, but are not limited to, tissue engineering scaffolds, drug delivery devices, tissue sealants, wound healing devices, construction materials, reinforcement materials, and any combinations thereof.

### Methods of Producing a Silk Fibroin-Comprising Composition or Article Described Herein.

Another aspect provided herein relates to methods of producing a silk fibroin-comprising composition or article described herein. The method comprises providing high molecular weight silk fibroin and forming a silk fibroin-comprising composition or article. In some embodiments, the high molecular weight silk fibroin can have an average molecular weight of at least about 200 kDa, and wherein no more than 30% of the silk fibroin can have a molecular weight of less than 100 kDa.

In accordance with embodiments of various aspects described herein, the high molecular weight silk fibroin can be produced by a process comprising degumming silk cocoons at a more gentle condition than a typical degumming condition known in the art. For example, in some embodiments, the high molecular weight silk fibroin can be produced by a process comprising degumming silk cocoons at a temperature of at least about 90° C. or higher (e.g., up to boiling temperature) for no more than 20 minutes, no more than 15 minutes, no more than 10 minutes, no more than 5 minutes, no more than 4 minutes, no more than 3 minutes, no more than 2 minutes, no more than 1 minute, no more than 30 seconds, or less. In some embodiments, the high molecular weight silk fibroin can be produced by a process comprising degumming silk cocoons at a temperature of at least about 90° C. for no more than 15 minutes, no more than 10 minutes, no more than 4 minutes, no more than 3 minutes or less.

In alternative embodiments, the high molecular weight silk fibroin can be produced by a process comprising degumming silk cocoons at a temperature in a range of about 50° C. to about 90°, including, for example, about 60° C. to about 90° C., about 60° C. to less than 90° C., or about 60° C. to about 80° C., for at least about 20 minutes or more, for example, including at least about 30 minutes, at least about 45 minutes, at least about 60 minutes, at least about 90 minutes or more. In some embodiments, the high molecular weight silk fibroin can be produced by a process comprising degumming silk cocoons at a temperature of about 60° C. to about 90° C. for at least about 30 minutes or longer, including, at least about 45 minutes, at least about 60 minutes or longer. In some embodiments, the high molecular weight silk fibroin can be produced by a process comprising degumming silk cocoons at a temperature of about 70° C. for at least about 30 minutes or longer, including, at least about 45 minutes, at least about 60 minutes or longer.

As used herein, the term “degumming” refers to heating silk cocoons in an aqueous solution to remove at least a portion of sericin from the silk cocoons. In one embodiment, the aqueous solution is about 0.02 M Na₂CO₃. In some embodiments, degumming can refer to heating silk cocoons in an aqueous solution to substantially remove sericin from native silk fibers. For example, the degummed silk fibers can have a sericin content of less than 10%, less than 8%, less than 6%, less than 5%, less than 4%, less than 3%, less than 2%, less than 1% or lower. In some embodiments, the degummed silk fibers can have a sericin content of less than 5% or lower.

The inventors have surprisingly discovered that degumming silk cocoons under more gentle conditions can be sufficient to substantially remove sericin from silk cocoons. Accordingly, in one aspect, methods for substantially removing sericin from silk cocoons are also provided herein. In some embodiments, the method of substantially removing sericin from silk cocoons comprises degumming silk cocoons at a temperature of at least about 90° C. or higher (e.g., up to boiling temperature) for a shorter period of time than what is known in the art to be required for substantially removing sericin. For example, in some embodiments, the method can comprise degumming silk cocoons at a temperature of at least about 90° C. or higher (e.g., up to boiling temperature) for no more than 20 minutes, no more than 15 minutes, no more than 10 minutes, no more than 5 minutes, no more than 4 minutes, no more than 3 minutes, no more than 2 minutes, no more than 1 minute, no more than 30 seconds, or less. In some embodiments, the method can comprise degumming silk cocoons at a temperature of at least about 90° C. for no more than 15 minutes, no more than 10 minutes, no more than 4 minutes, no more than 3 minutes or less.

Alternatively, the method of substantially removing sericin from silk cocoons can comprise degumming silk cocoons at a temperature of no more than 90° C. for a longer period of time. For example, the method can comprise degumming silk cocoons at a temperature in a range of about 50° C. to about 90°, including, for example, about 60° C. to about 90° C., about 60° C. to less than 90° C., or about 60° C. to about 80° C., for at least about 20 minutes or more, for example, including at least about 30 minutes, at least about 45 minutes, at least about 60 minutes, at least about 90 minutes or more. In some embodiments, the method can comprise degumming silk cocoons at a temperature of about 60° C. to about 90° C. for at least about 30 minutes or longer, including, at least about 45 minutes, at least about 60 minutes or longer. In some embodiments, the method can comprise degumming silk cocoons at a temperature of about 70° C. for at least about 30 minutes or longer, including, at least about 45 minutes, at least about 60 minutes or longer.

After degumming, the cocoons are rinsed, for example, with water to extract the sericin proteins. To prepare a silk fibroin solution, the extracted silk can be dissolved in an aqueous salt solution. Salts that can be used for this purpose include lithium bromide, lithium thiocyanate, calcium nitrate, or other chemicals capable of solubilizing silk. In some embodiments, the extracted silk can be dissolved in about 8M-12 M LiBr solution. The salt can be consequently removed using, for example, dialysis.

If necessary, the silk fibroin solution can then be concentrated using, for example, dialysis against a hygroscopic polymer, for example, PEG, a polyethylene oxide, amylose or sericin. In some embodiments, the PEG is of a molecular weight of 8,000-10,000 g/mol and has a concentration of about 10% to about 50% (w/v). A slide-a-lyzer dialysis cassette (Pierce, MW CO 3500) can be used. However, any dialysis system can be used. The dialysis can be performed for a time period sufficient to result in a final concentration of aqueous silk solution between about 10% to about 30%. In most cases dialysis for 2-12 hours can be sufficient. See, for example, International Patent Application Publication No. WO 2005/012606, the content of which is incorporated herein by reference in its entirety.

Alternatively, the silk fibroin solution can be produced using organic solvents. Such methods have been described, for example, in Li, M., et al., J. Appl. Poly Sci. 2001, 79, 2192-2199; Min, S., et al. Sen'I Gakkaishi 1997, 54, 85-92; Nazarov, R. et al., Biomacromolecules 2004 May-June; 5(3):718-26, content of all which is incorporated herein by reference in their entirety. An exemplary organic solvent that can be used to produce a silk solution includes, but is not limited to, hexafluoroisopropanol (HFIP). See, for example, International Application No. WO2004/000915, content of which is incorporated herein by reference in its entirety.

In some embodiments, the silk fibroin solution can comprise an organic solvent, e.g., HFIP. In some other embodiments, the solution is free or essentially free of organic solvents, i.e., solvents other than water.

In some embodiments, the silk fibroin solution can be further processed to isolate silk fibroin having a specific high molecular weight, or within a specific high molecular weight distribution. Methods for purifying polymers with a desirable molecular weight or a molecular weight distribution are known in the art, e.g., but not limited to, gel permeation chromatography, and can be used to isolate silk fibroin with a specific molecular weight or molecular weight distribution.

Generally, any amount of high molecular weight silk fibroin can be present in the solution. For example, amount of silk in the solution or the composition prepared therefrom can range from about 0.1% (w/v or w/w) to about 50% (w/v or w/w) of silk, e.g., silk fibroin. In some embodiments, the amount of silk in the solution or the composition prepared therefrom can be from about 0.2% (w/v or w/w) to about 35% (w/v or w/w), from about 0.5% (w/v or w/w) to about 30% (w/v or w/w), from about 0.5% (w/v or w/w) to about 25% (w/v or w/w), from about 0.5% (w/v or w/w) to about 20% (w/v or w/w), or from about 0.5% (w/v or w/w) to about 10% (w/v or w/w). In one embodiment, the amount of silk in the solution or the composition prepared therefrom can be from about 0.1% (w/v or w/w) to about 10% (w/v or w/w). Depending on applications, degumming time, molecular weights of silk fibroin, and/or methods of making a solid-state silk fibroin, the amount of the high molecular weight silk fibroin can be optimized accordingly. For example, as shown in Example 5, the concentration of the high molecular weight silk fibroin solution can be at least about 10% (w/v or w/w), at least about 15% (w/v or w/w), at least about 20% (w/v or w/w) or more, in order to reach minimum viscosity requirement for gel spinning to form a tubular silk fibroin structure. In another instance as shown in Example 4, the concentration of the high molecular weight silk fibroin solution can be as low as 0.5% (w/v or w/w) to form a silk fibroin scaffold. Exact amount of silk in the silk solution can be determined by drying a known amount of the silk solution and measuring the mass of the residue to calculate the solution concentration.

Without wishing to be bound by theory, molecular weight and/or concentrations of silk fibroin can, in part, affect mechanical and/or degradation properties of the resulting silk fibroin-based compositions and/or article. Thus, in some embodiments, the method of producing a silk fibroin-based composition and/or article can comprise selecting high molecular weight silk fibroin at a pre-determined concentration for a desirable mechanical and/or degradation properties of the resulting silk fibroin-based composition and/or article. In some embodiments, the method can comprise controlling the degumming temperature and/or time as described herein in order to obtain the selected high molecular weight silk fibroin.

As silk fibroin can generally stabilize active agents, some embodiments of the composition or solid-state silk fibroin described herein can be used to encapsulate and/or deliver at least one an active agent. In these embodiments, at least one active agent can be dispersed into a high molecular weight silk fibroin solution. Non-limiting examples of the active agents can include cells, proteins, peptides, nucleic acids, nucleic acid analogs, nucleotides or oligonucleotides, peptide nucleic acids, aptamers, antibodies or fragments or portions thereof, antigens or epitopes, hormones, hormone antagonists, growth factors or recombinant growth factors and fragments and variants thereof, cell attachment mediators, cytokines, enzymes, antibiotics or antimicrobial compounds, viruses, toxins, therapeutic agents and prodrugs thereof, small molecules, and any combinations thereof.

In some embodiments, the silk fibroin solution can further comprise at least one additive as described herein.

In some embodiments, at least one active agent and/or additive described herein can be added to the silk fibroin solution before further processing into a solid-state silk fibroin described herein. In some embodiments, the active agent and/or additive can be dispersed homogeneously or heterogeneously within the silk fibroin, dispersed in a gradient, e.g., using the carbodiimide-mediated modification method described in the U.S. Patent Application No. US 2007/0212730.

In some embodiments, the solid-state silk fibroin can be first formed and then contacted with (e.g., dipped into or incubated with) at least one active agent and/or additive. In some embodiments, at least one active agent and/or additive described herein can be coated on an exposed surface of the solid-state silk fibroin upon the contacting. In some embodiments, at least one active agent and/or additive described here can diffuse into the solid-state silk fibroin upon the contacting.

The high molecular weight silk fibroin solution can be used directly to form a solid-state silk fibroin. For example, the silk fibroin solution can be treated to induce a conformational change in the silk fibroin therein, thereby forming a solid-state silk fibroin. In some embodiments, the silk solution can be placed in a mold prior to inducing conformational change in the silk fibroin therein. Alternatively, the resulting solid-state silk fibroin can be subsequently dissolved or be reduced to particles or powder, e.g., by grinding, milling, cutting, pulverizing, and any combinations thereof, to form a silk fibroin solution or powder for use in regenerating another solid-state silk fibroin. In some embodiments where the high molecular silk fibroin is provided as particles or powder, a solid-state silk fibroin can be formed, e.g., by molding such as sintering, metal injection molding and/or powder compaction. In one embodiment, the high molecular silk fibroin powder can be used to form a solid-state silk fibroin by powder compaction as described in U.S. Provisional Application No. 61/671,375 filed Jul. 13, 2012. Without wishing to be bound by a theory, forming a solid-state silk fibroin and dissolving it in a solvent or reducing it into particles or powder can allow one to obtain silk solutions of higher concentrations, or regenerate a new solid-state silk fibroin of higher density.

The solid-state silk fibroin can be in any form, shape or size. Examples of a solid-state silk fibroin include, but are not limited to, a film, a sheet, a gel or hydrogel, a mesh, a mat, a non-woven mat, a fabric, a scaffold, a tube, a slab or block, a fiber, a particle, powder, a 3-dimensional construct, an implant, a foam or a sponge, a needle, a high density material, a lyophilized material, and any combinations thereof.

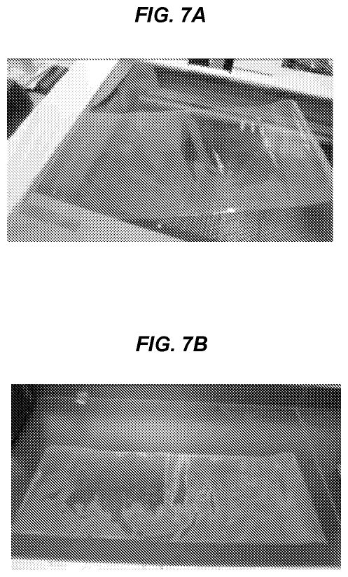

In some embodiments, the solid-state silk fibroin can be in the form of a film, e.g., a silk fibroin film. As used herein, the term “film” refers to a flat structure or a thin flexible substrate that can be rolled to form a tube. In some embodiments, the term “film” can also refer to a tubular flexible structure. It is to be noted that the term “film” is used in a generic sense to include a web, film, sheet, laminate, or the like. In some embodiments, the film can be a patterned film, e.g., nanopatterned film. Exemplary methods for preparing silk fibroin films are described in, for example, WO 2004/000915 and WO 2005/012606, content of both of which is incorporated herein by reference in its entirety. In some embodiments, a silk fibroin film can be produced by drying a silk fibroin solution on a substrate, e.g., a petri dish or a piece of acrylic. The resulting silk film can be further annealed, e.g., by water annealing or water vapor annealing, and then the resulting film can then be removed. As shown in FIGS. 7A and 7B, larger and higher quality silk films can be produced using high molecular weight silk fibroin. The mechanical toughness of these films can allow them to be handled without film failure and rolled into a tight spiral.

In some embodiments, the solid-state silk fibroin can be in the form of a silk particle, e.g., a silk nanosphere or a silk microsphere. As used herein, the term “particle” includes spheres; rods; shells; and prisms; and these particles can be part of a network or an aggregate. Without limitations, the particle can have any size from nm to millimeters. As used herein, the term “microparticle” refers to a particle having a particle size of about 1 μm to about 1000 μm. As used herein, the term “nanoparticle” refers to particle having a particle size of about 0.1 nm to about 1000 nm.

In some embodiments, the solid-state silk fibroin can be in the form of a gel or hydrogel. The term “hydrogel” is used herein to mean a silk-based material which exhibits the ability to swell in water and to retain a significant portion of water within its structure without dissolution. Methods for preparing silk fibroin gels and hydrogels are well known in the art. Methods for preparing silk fibroin gels and hydrogels include, but are not limited to, sonication, vortexing, pH titration, exposure to electric field, solvent immersion, water annealing, water vapor annealing, and the like. Exemplary methods for preparing silk fibroin gels and hydrogels are described in, for example, WO 2005/012606, content of which is incorporated herein by reference in its entirety. As shown in Example 3, high molecular weight silk fibroin (e.g., at a concentration of about 8% (w/v) can be used to form a higher-density and mechanically stiffer gel by electrogelation using a lower DC voltage, as compared to using lower molecular weight silk fibroin.

In some embodiments, the solid-state silk fibroin can be in the form of a foam or a sponge. Methods for preparing silk fibroin foams or sponges are well known in the art. In some embodiments, the foam or sponge is a patterned foam or sponge, e.g., nanopatterned foam or sponge. Exemplary methods for preparing silk foams and sponges are described in, for example, WO 2004/000915, WO 2004/000255, and WO 2005/012606, content of all of which is incorporated herein by reference in its entirety. Without wishing to be bound by theory, high molecular weight silk fibroin can provide a more continuous and tougher network of bonded silk between and around each pore in a foam construct, thus creating a foam construct with improved mechanical performance to a traditional cast silk foam using lower molecular weight silk fibroin. In some embodiments, a foam can be produced by using a freeze-drying process. Layered foams can be produced by applying at least one layer of high molecular weight silk fibroin solution on top of another frozen layers, and allowing the newly applied layer to freeze. The final frozen structure can then be placed in a lyophilizer where the structure is freeze-dried and water molecules are extracted from the construct. In some embodiments, the high molecular weight silk fibroin can form a foam that is not as susceptible to water dissolution.

In some embodiments, the solid-state silk fibroin can be in the form of a cylindrical matrix, e.g., a silk tube. The silk tubes can be made using any method known in the art. For example, tubes can be made using molding, dipping, electrospinning, gel spinning, and the like. Gel spinning is described in Lovett et al. (Biomaterials, 29(35):4650-4657 (2008)) and the construction of gel-spun silk tubes is described in PCT application no. PCT/US2009/039870, filed Apr. 8, 2009, content of both of which is incorporated herein by reference in their entirety. Construction of silk tubes using the dip-coating method is described in PCT application no. PCT/US2008/072742, filed Aug. 11, 2008, content of which is incorporated herein by reference in its entirety. Construction of silk fibroin tubes using the film-spinning method is described in PCT application No. PCT/US2013/030206, filed Mar. 11, 2013 and U.S. Provisional application No. 61/613,185, filed Mar. 20, 2012.

In some embodiments, the solid-state silk fibroin can be in the form of a fiber. A silk fibroin fiber can be formed from a high molecular weight silk fibroin solution with any methods known in the art, including, but not limited to, molding, machining, drawing, eletrogelation, electrospinning, or any combinations thereof. In some embodiments, a silk fibroin fiber can be formed by drying (e.g., by freezing) a silk fibroin solution in a mold that is in a form of an elongated tube. See, e.g., the International Patent Application No. WO 2012/145594, the content of which is incorporated herein by reference, for exemplary methods that can be modified to make a silk fibroin fiber described herein. In some embodiments, a silk fibroin fiber can be formed by drawing a fiber from a viscous high molecular weight silk fibroin solution that has been processed by electrogelation. See, e.g., the International Patent Application No. WO 2011/038401, the content of which is incorporated herein by reference, for exemplary methods that can be modified to making a silk fibroin fiber described herein. Electrospun silk materials, such as fibers, and methods for preparing the same are described, for example in WO2011/008842, content of which is incorporated herein by reference in its entirety. Micron-sized silk fibers (e.g., 10-600 μm in size) and methods for preparing the same are described, for example in Mandal et al., Proc Natl Acad Sci USA. 2012 May 15; 109(20):7699-704 “High-strength silk protein scaffolds for bone repair;” and PCT application no. PCT/US13/35389, filed Apr. 5, 2013, content of all of which is incorporated herein by reference.

In some embodiments, it can be desirable to have the solid-state silk fibroin to be porous as described earlier. Too high porosity can generally yield a solid-state silk fibroin and thus the resulting network thereof with lower mechanical properties, but too low porosity can affect the release of an active agent embedded therein, if any. One of skill in the art can adjust the porosity accordingly, based on a number of factors such as, but not limited to, desired release rates, molecular size and/or diffusion coefficient of the active agent, and/or concentrations and/or amounts of silk fibroin in a solid-state silk fibroin.

The porous solid-state silk fibroin can have any pore size as described earlier. Methods for forming pores in a solid-state silk fibroin are known in the art and include, but are not limited, porogen-leaching methods, freeze-drying methods, and/or gas-forming method. Exemplary methods for forming pores in a silk-based material are described, for example, in U.S. Pat. App. Pub. Nos.: US 2010/0279112 and US 2010/0279112; U.S. Pat. No. 7,842,780; and WO2004062697, content of all of which is incorporated herein by reference in its entirety.

Without wishing to be bound by theory, in some embodiments, long chains of high molecular weight silk fibroin can entangle with each other and hinder the packing of silk fibroin during formation of a solid-state silk fibroin. Accordingly, in some embodiments, it can be desirable to improve packing and/or molecular alignment of silk fibroin, which can facilitate chain-to-chain bonds, leading to crystallinity in silk fibroin and/or more mechanically robust properties. Thus, in these embodiments, forming a solid state silk fibroin from a high molecular weight silk fibroin composition can comprise inducing molecular/chain alignment and/or improving packing of silk fibroin. In some embodiments, the packing of silk fibroin can be improved by blending in some shorter chain fibroin (e.g., low molecular weight silk fibroin) into a high molecular weight silk fibroin solution. In other embodiments, a surfactant can be used to allow for chain mobility until post-process stabilization of silk fibroin chains into higher order conformation, e.g., beta sheet formation. In some embodiments, the packing of silk fibroin can be controlled by increasing pH of the high molecular weight silk fibroin solution. In other embodiments, molecular alignment and/or packing of silk fibroin can be induced by exposing a high molecular weight silk fibroin solution to vibration (e.g., sonication and/or vortexing as described in the International Appl. Nos. WO/2008/150861 and WO/2011005381, the contents of which are incorporated herein by reference), or casting the high molecular weight silk fibroin solution on a surface. In some embodiments, molecular alignment and/or packing of silk fibroin can be induced by exposing a high molecular weight silk fibroin solution to an electric field (e.g., as described in the International Appl. No. WO/2010/036992, the content of which is incorporated herein by reference).

After formation of the solid-state silk fibroin, in some embodiments, the solid-state silk fibroin can be further subjected to a post-treatment. A post-treatment can include any process that can alter a material or physical property of the solid-state silk fibroin. For example, in some embodiments, the solid-state silk fibroin can be further processed into a variety of desired shapes. Examples of such processing methods include, but are not limited to, machining, turning (lathe), rolling, thread rolling, drilling, milling, sanding, punching, die cutting, blanking, broaching, and any combinations thereof.

In some embodiments, the solid-state silk fibroin can be subjected to a post-treatment that can increase its mechanical performance. For example, in some embodiments, the solid-state silk fibroin, e.g., a film or a fiber can be further subjected to stretching or drawing over steam. The stretch or draw ratio (i.e., difference in length between before and after drawing divided by original length before drawing) can depend on the material property of the solid-state silk fibroin. In some embodiments, the stretch or draw ratio can range from about 0.1 to about 10, or from about 0.5 to about 5, or from about 1 to about 4. Without wishing to be bound by theory, stretching or drawing the solid-state silk fibroin, e.g., a film, or a fiber, can provide additional alignment of silk fibroin molecules, and thus yield a stronger and more ductile silk fibroin material. Example 2 shows effect of steam drawing of a silk fibroin film on improved mechanical properties of the drawn film.

In some embodiments, a post-treatment method can be applied to the solid-state silk fibroin to further induce a conformational change in the silk fibroin as described herein. In some embodiments, a conformational change in the silk fibroin can increase crystallinity of the silk fibroin, e.g., silk II beta-sheet crystallinity.

In some embodiments, the composition and/or solid-state silk fibroin described herein can be sterilized. Sterilization methods for biomaterials are well known in the art, including, but not limited to, gamma or ultraviolet radiation, autoclaving (e.g., heat/steam); alcohol sterilization (e.g., ethanol and methanol); and gas sterilization (e.g., ethylene oxide sterilization).

Further, the silk fibroin-based material described herein can take advantage of the many techniques developed to functionalize silk fibroin (e.g., active agents such as dyes and sensors). See, e.g., U.S. Pat. No. 6,287,340, Bioengineered anterior cruciate ligament; WO 2004/000915, Silk Biomaterials & Methods of Use Thereof; WO 2004/001103, Silk Biomaterials & Methods of Use Thereof; WO 2004/062697, Silk Fibroin Materials & Use Thereof; WO 2005/000483, Method for Forming inorganic Coatings; WO 2005/012606, Concentrated Aqueous Silk Fibroin Solution & Use Thereof; WO 2011/005381, Vortex-Induced Silk fibroin Gelation for Encapsulation & Delivery; WO 2005/123114, Silk-Based Drug Delivery System; WO 2006/076711, Fibrous Protein Fusions & Uses Thereof in the Formation of Advanced Organic/Inorganic Composite Materials; U.S. Application Pub. No. 2007/0212730, Covalently immobilized protein gradients in three-dimensional porous scaffolds; WO 2006/042287, Method for Producing Biomaterial Scaffolds; WO 2007/016524, Method for Stepwise Deposition of Silk Fibroin Coatings; WO 2008/085904, Biodegradable Electronic Devices; WO 2008/118133, Silk Microspheres for Encapsulation & Controlled Release; WO 2008/108838, Microfluidic Devices & Methods for Fabricating Same; WO 2008/127404, Nanopatterned Biopolymer Device & Method of Manufacturing Same; WO 2008/118211, Biopolymer Photonic Crystals & Method of Manufacturing Same; WO 2008/127402, Biopolymer Sensor & Method of Manufacturing Same; WO 2008/127403, Biopolymer Optofluidic Device & Method of Manufacturing the Same; WO 2008/127401, Biopolymer Optical Wave Guide & Method of Manufacturing Same; WO 2008/140562, Biopolymer Sensor & Method of Manufacturing Same; WO 2008/127405, Microfluidic Device with Cylindrical Microchannel & Method for Fabricating Same; WO 2008/106485, Tissue-Engineered Silk Organs; WO 2008/140562, Electroactive Biopolymer Optical & Electro-Optical Devices & Method of Manufacturing Same; WO 2008/150861, Method for Silk Fibroin Gelation Using Sonication; WO 2007/103442, Biocompatible Scaffolds & Adipose-Derived Stem Cells; WO 2009/155397, Edible Holographic Silk Products; WO 2009/100280, 3-Dimensional Silk Hydroxyapatite Compositions; WO 2009/061823, Fabrication of Silk Fibroin Photonic Structures by Nanocontact Imprinting; WO 2009/126689, System & Method for Making Biomaterial Structures.

In some embodiments, the silk fibroin-based material can include plasmonic nanoparticles to form photothermal elements, e.g., by adding plasmonic particles into a magnetic silk solution and forming a silk fibroin-based material therefrom. This approach takes advantage of the superior doping characteristics of silk fibroin. Thermal therapy has been shown to aid in the delivery of various agents, see Park et al., Effect of Heat on Skin Permeability, 359 Intl. J. Pharm. 94 (2008). In one embodiment, short bursts of heat on very limited areas can be used to maximize permeability with minimal harmful effects on surrounding tissues. Thus, plasmonic particle-doped silk fibroin matrices can add specificity to thermal therapy by focusing light to locally generate heat only via the silk fibroin matrices. In some embodiments, the silk fibroin matrices can include photothermal agents such as gold nanoparticles.

### Inducing a Conformation Change in Silk Fibroin

Inducing a conformational change in silk fibroin can facilitate formation of a solid-state silk fibroin and/or make the silk fibroin at least partially insoluble. Without wishing to be bound by a theory, in some embodiments, the induced conformational change can increase the crystallinity of the silk fibroin, e.g., silk II beta-sheet crystallinity, which can in turn modulate physical properties of silk fibroin (e.g., mechanical strength, degradability and/or solubility). Further, inducing formation of beta-sheet conformation structure in silk fibroin can prevent silk fibroin from contracting into a compact structure and/or forming an entanglement. R example, the conformational change in silk fibroin can be induced by one or more methods, including but not limited to, controlled slow drying (Lu et al., 10 Biomacromolecules 1032 (2009)); water annealing (Jin et al., 15 Adv. Funct. Mats. 1241 (2005); Hu et al., 12 Biomacromolecules 1686 (2011)); stretching (Demura & Asakura, 33 Biotech & Bioengin. 598 (1989)); compressing; solvent immersion, including methanol (Hofmann et al., 111 J Control Release. 219 (2006)), ethanol (Miyairi et al., 56 J. Fermen. Tech. 303 (1978)), glutaraldehyde (Acharya et al., 3 Biotechnol J. 226 (2008)), and 1-ethyl-3-(3-dimethyl aminopropyl) carbodiimide (EDC) (Bayraktar et al., 60 Eur J Pharm Biopharm. 373 (2005)); pH adjustment, e.g., pH titration and/or exposing a silk-based material to an electric field (see, e.g., U.S. Patent App. No. US2011/0171239); heat treatment; shear stress (see, e.g., International App. No.: WO 2011/005381), ultrasound, e.g., sonication (see, e.g., U.S. Patent Application Publication No. U.S. 2010/0178304, and International Patent Application No. WO2008/150861); constraint-drying (see, e.g., International Patent Application No. WO 2011/008842); and any combinations thereof. Content of all of the references listed above is incorporated herein by reference in their entirety.

As used herein, the term “constraint-drying” refers to a process where the silk material is dried while being constrained, such that it dries while undergoing a drawing or stretching force. Without wishing to be bound by theory, as water molecules evaporate, hydrophobic domains at the surface substrate and throughout the bulk region of the protein can initiate the loss of free volume from the interstitial space of the non-woven cast and within bulk region of the material. The loss of free volume can thus cause the material to contract. An exemplary method of constraint-drying a silk fibroin-based material can employ a magnetic field to maintain a silk fibroin-based material being stretched until it becomes naturally or blown dry.

In some embodiments, the conformation of the silk fibroin can be altered by water annealing. Without wishing to be bound by a theory, it is believed that physical temperature-controlled water vapor annealing (TCWVA) provides a simple and effective method to obtain refined control of the molecular structure of silk biomaterials. The silk materials can be prepared with control of crystallinity, from a low content using conditions at 4° C. (a helix dominated silk I structure), to highest content of ˜60% crystallinity at 100° C. (β-sheet dominated silk II structure). This physical approach covers the range of structures previously reported to govern crystallization during the fabrication of silk materials, yet offers a simpler, green chemistry, approach with tight control of reproducibility. Temperature controlled water vapor annealing is described, for example, in Hu et al., Regulation of Silk Material Structure By Temperature Controlled Water Vapor Annealing, Biomacromolecules, 2011, 12(5): 1686-1696, content of which is incorporated herein by reference in its entirety.

In some embodiments, alteration in the conformation of the silk fibroin can be induced by immersing in alcohol, e.g., methanol, ethanol, etc. The alcohol concentration can be at least 10%, at least 20%, at least 30%, at least 40%, at least 50%, at least 60%, at least 70%, at least 80%, at least 90% or 100%. In some embodiment, alcohol concentration is 100%. If the alteration in the conformation is by immersing in a solvent, the silk composition can be washed, e.g., with solvent/water gradient to remove any of the residual solvent that is used for the immersion. The washing can be repeated one, e.g., one, two, three, four, five, or more times.

Alternatively, the alteration in the conformation of the silk fibroin can be induced with shear stress (see, e.g., International Pat. App. No. WO/2011005381, and U.S. patent application Ser. No. 12/934,666, the content of each of which is incorporated herein by reference). The shear stress can be applied, for example, by passing the silk composition through a needle. Other methods of inducing conformational changes include applying an electric field, applying pressure, or changing the salt concentration.

The treatment time for inducing the conformational change can be any period of time to provide a desired silk II (beta-sheet crystallinity) content. In some embodiments, the treatment time can range from about 1 hour to about 12 hours, from about 1 hour to about 6 hours, from about 1 hour to about 5 hours, from about 1 hour to about 4 hours, or from about 1 hour to about 3 hours. In some embodiments, the treatment time can range from about 2 hours to about 4 hours or from 2.5 hours to about 3.5 hours.

When inducing the conformational change is by solvent immersion, treatment time can range from minutes to hours. For example, immersion in the solvent can be for a period of at least about 15 minutes, at least about 30 minutes, at least about 1 hour, at least about 2 hours, at least 3 hours, at least about 6 hours, at least about 18 hours, at least about 12 hours, at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, or at least about 14 days. In some embodiments, immersion in the solvent can be for a period of about 12 hours to about seven days, about 1 day to about 6 days, about 2 to about 5 days, or about 3 to about 4 days.

After the treatment to induce the conformational change, silk fibroin in the silk composition can comprise a silk II beta-sheet crystallinity content of at least about 5%, at least about 10%, at least about 20%, at least about 30%, at least about 40%, at least about 50%, at least about 60%, at least about 70%, at least about 80%, at least about 90%, or at least about 95% but not 100% (i.e., all the silk is present in a silk II beta-sheet conformation). In some embodiments, silk fibroin in the silk composition is present completely in a silk II beta-sheet conformation, i.e., 100% silk II beta-sheet crystallinity.

Exemplary Applications and/or Uses of Compositions or Silk Fibroin Articles Described Herein

Different embodiments of solid-state silk fibroin or silk fibroin-based materials made from high molecular weight silk fibroin described herein can be adapted for use in various applications, and/or in forming novel compositions and/or articles. Modulating molecular weight of silk fibroin, concentration of silk fibroin, and/or packing and/or crystallinity of silk fibroin can yield silk fibroin-based compositions and/or articles of different structural, mechanical and/or degradation properties. For example, long silk fibroin chains (high molecular weight silk fibroin) with poor packing (e.g., due to entanglements of long chains) and/or low crystallinity (e.g., low higher-order conformation such as low beta-sheet content) can yield silk fibroin-based compositions and/or articles with weaker mechanical strength and/or faster degradation, as compared to long silk fibroin chains (high molecular weight silk fibroin) with great packing (e.g., where the silk fibroin molecules are aligned) and/or crystallinity.

High molecular weight silk fibroin can be used at any concentrations as described herein for desirable structural, mechanical and/or degradation properties. For example, Example 5 shows that silk tubes made from lower concentrations of high molecular weight silk fibroin can have larger pore sizes and/or higher porosity, and thus degrade faster than their lower molecular weight counterparts which require higher concentrations in order to achieve a minimum viscosity for gel-spinning Without wishing to be bound by theory, it is possible that the larger pore sizes of the high molecular weight silk fibroin tubes allow for greater fluid transport and/or enzyme exposure, thus facilitating its more rapid degradation. Accordingly, high molecular weight silk fibroin can be used to fabricate novel silk fibroin-based compositions and/or articles with material properties (e.g., combination of mechanical and degradation properties) that cannot be achieved using lower molecular weight counterparts otherwise.

Bioresorbable Implants:

In some embodiments, high molecular weight silk fibroin can be used to form bioresorbable implants, such as bioresorbable silk tubes, e.g., for blood vessel repair/replacement, and/or bioresorbable silk scaffold such as a tissue scaffold or wound dressing. By “bioresorbable” is meant the ability of a material to be resorbed or remodeled in vivo. The resorption process involves degradation and elimination of the original implant material through the action of body fluids, enzymes or cells. The resorbed materials can be used by the host in the formation of new tissue, or it can be otherwise re-utilized by the host, or it can be excreted. The bioresorbable silk fibroin article described herein can have a resorption half-life ranging from a few hours to weeks to months. In some embodiments, the resorption half-life of the bioresorbable silk fibroin article described herein can be in a range of about 6 hours to about 4 weeks, about 12 hours to about 3 weeks, about 24 hours to about 2 weeks. In some embodiments, the resorption half-life of the bioresorbable silk fibroin article described herein can be at least about 1 months, at least about 2 months, at least about 3 months, at least about 4 months, at least about 5 months, at least about 6 months, at least about 7 months, at least about 8 months, at least about 9 months, at least about 10 months, at least about 11 months, at least about 12 months or longer. In some embodiments, the resorption half-life of the bioresorbable silk fibroin article described herein can be about 1 month to about 3 months, or about 3 months to about 6 months, or about 6 months to about 12 months.

Tissue Scaffolds:

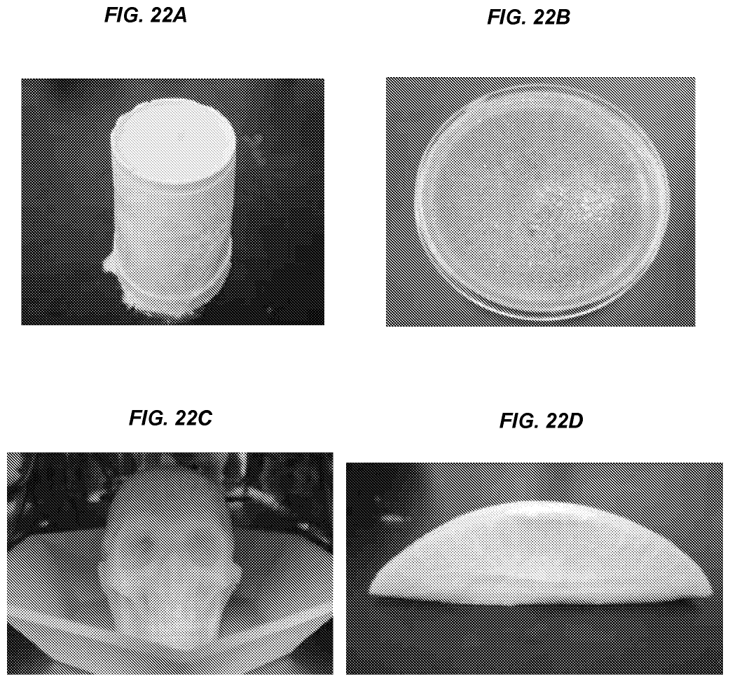

In some embodiments, high molecular weight silk fibroin can be used to form a tissue scaffold. Scaffolds can be made using low concentration (e.g., ˜0.5%-˜15%) of high molecular weight silk fibroin e.g., to create high porosity with large pores in order to mimic a physiological tissue architecture, while maintaining structural integrity. In some embodiments, scaffolds can be made using low concentration (e.g., ˜0.5%-˜15%) of high molecular weight silk fibroin to form a softer construct while maintaining structural integrity, e.g., a breast implant as shown in FIG. 22D. Alternatively, scaffolds can be made using high concentration of high molecular weight silk fibroin for enhanced mechanical performance. The mechanical robustness of the silk fibroin scaffolds formed from high molecular weight silk fibroin can be used, for example, in void filling, stabilization and/or repair of mechanically loaded tissues, e.g., but not limited to bones.

In some embodiments, the silk fibroin scaffold can have compressive strength, compressive toughness and compressive elastic modulus values approximate to those of healthy human bone and enables load-bearing. Without wishing to be bound by a theory, load-bearing properties can also prevent unwanted resorption of adjacent bone resulting from high local stress concentration or stress-shielding.

Compressive toughness is the capacity of a material to resist fracture when subjected to axially directed pushing forces. By definition, the compressive toughness of a material is the ability to absorb mechanical (or kinetic) energy up to the point of failure. Toughness is measured in units of joules per cubic meter (Jm⁻³) and can be measured as the area under a stress-strain curve. In some embodiments, the silk fibroin scaffold described herein can have a compressive toughness of about 1 kJ m⁻³ to about 20 kJm⁻³ or about 1 kJm⁻³ to approximately 5 kJm³ at 6% strain as measured by the J-integral method. In one embodiment, the silk fibroin scaffold can have a compressive toughness of about 1.3 kJm⁻³, which is the approximate compressive toughness of healthy bone.

Compressive strength is the capacity of a material to withstand axially directed pushing forces. By definition, the compressive strength of a material is that value of uniaxial compressive stress reached when the material fails completely. A stress-strain curve is a graphical representation of the relationship between stress derived from measuring the load applied on the sample (measured in MPa) and strain derived from measuring the displacement as a result of compression of the sample. The ultimate compressive strength of the material can depend upon the target site of implantation. For example, if the material is for placement next to osteoporotic cancellous bone, to avoid high stress accumulation and stress shielding, the material can comprise a compressive strength (stress to yield point) of approximately 0.1 MPa to approximately 2 MPa. If the material is intended for placement next to healthy cancellous bone, the material can comprise an ultimate compressive strength (stress to yield point) of approximately 5 MPa. Alternatively, if the material is intended for placement next to cortical bone, the material can comprise an ultimate compressive strength (stress to yield point) of at least 40 MPa.

In some embodiments, the silk fibroin scaffold described herein can comprise an ultimate compressive strength (stress to yield point) of at least 5 MPa, at least 10 MPa, at least 15 MPa, at least 20 MPa, at least 25 MPa, at least 30 MPa, at least 35 MPa, at least 40 MPa, at least 45 MPa, at least 50 MPa, at least 55 MPa, at least 60 MPa, at least 65 MPa, at least 70 MPa, at least 75 MPa, at least 80 MPa, at least 85 MPa, at least 90 MPa, at least 95 MPa, at least 100 MPa, at least 105 MPa, at least 110 MPa, at least 115 MPa, at least 120 MPa, at least 125 MPa, at least 130 MPa, at least 135 MPa, at least 140 MPa, at least 145 MPa, at least 150 MPa, or at least 155 MPa, for example, at 5% strain.

Compressive elastic modulus is the mathematical description of the tendency of a material to be deformed elastically (i.e. non-permanently) when a force is applied to it. The Young's modulus (E) describes tensile elasticity, or the tendency of a material to deform along an axis when opposing forces are applied along that axis; it is defined as the ratio of tensile stress to tensile strain (measured in MPa) and is otherwise known as a measure of stiffness of the material. The elastic modulus of an object is defined as the slope of the stress-strain curve in the elastic deformation region. The silk fibroin scaffold described herein can comprise a compressive elastic modulus of between approximately 100 MPa and approximately 5,000 MPa GPa at 5% strain. In some embodiments, the silk fibroin scaffold described herein can comprise a compressive elastic modulus of between approximately 200 MPa and 750 MPa, between approximately 250 MPa and 700 MPa, between approximately 300 MPa and 650 MPa, between approximately 400 MPa and 600 MPa, or between approximately 450 MPa and 550 MPa, for example, at 5% strain.

In some embodiments, the silk fibroin scaffold described herein can have a mean compressive elastic modulus of about 525 MPa. In some embodiments, the silk fibroin scaffold described herein can comprise a compressive elastic modulus of at least 100 MPa, at least 150 MPa, at least 200 MPa, at least 250 MPa, at least 300 MPa, at least 350 MPa, at least 400 MPa, at least 450 MPa, at least 500 MPa, or at least 525 MPa.

Not only can high molecular weight silk fibroin be used to produce high-strength materials, but high molecular weight silk fibroin can also be used to make a three-dimensional construct with a complex geometry, for example a skull as shown in FIG. 22C, and other medical devices such as bone screws and plates.

Wound Dressing/Tissue Sealants:

Without wishing to be bound by theory, high molecular weight silk fibroin in solution can self-assemble faster than lower molecular weight silk fibroin. Thus, high molecular weight silk fibroin can form a gel faster than when lower molecular weight silk fibroin is used. The faster gelation of high molecular weight silk fibroin in solution can be desired in applications where rapid gelation is needed, e.g., for treatment of a wound, e.g., to stop bleeding. In one embodiment, the high molecular weight silk fibroin can be provided as powder, which can be reconstituted in solution when it is ready for use, e.g., to apply to a wound.

Thin-Walled Three-Dimensional Constructs (Hollow Constructs):

The longer silk fibroin chains (high molecular weight silk fibroin) can provide a more continuous and tougher network of bonded silk, thus providing enhanced mechanical performance even in a thin-walled or hollow structure. For example, FIG. 22A shows a large, fairly thin-walled cup made from high molecular weight silk fibroin. Without wishing to be limiting, in some embodiments, high molecular weight silk fibroin can be used to form any hollow construct such as hollow organs, e.g., but not limited to stomach, intestine, heart, and urinary bladder.

Reinforcement Materials:

In another embodiment, high molecular weight silk fibroin can be used to form reinforcement materials such as silk fibers, silk microfibers and/or silk particles that can be added to enhance the mechanical property (e.g., increased stiffness) of a bulk material. In some embodiments, a solid-state silk fibroin made from high molecular weight silk fibroin can be reduced (e.g., by milling or grinding) into silk fibroin particles or powder.

Flexible Electronics:

In some embodiments, high molecular weight silk fiborin can be used to form a substrate for flexible electronics (Hwang S.-W., et al., Science, 2012, 377 (6102): 1640-1644). As shown in FIGS. 7A and 7B, large and high quality (e.g., mechanically strong and tough) silk films can be produced using silk fibroin of high molecular weights. In some embodiments, the mechanical toughness of the high molecular weight silk fibroin film can give the film a “plastic-like” feel and allow it to be handled without film failure and rolled into a tight spiral. In some embodiments, the surface of the film can comprise small features such as an optical pattern, e.g., but not limited to a diffraction pattern.

Sutures:

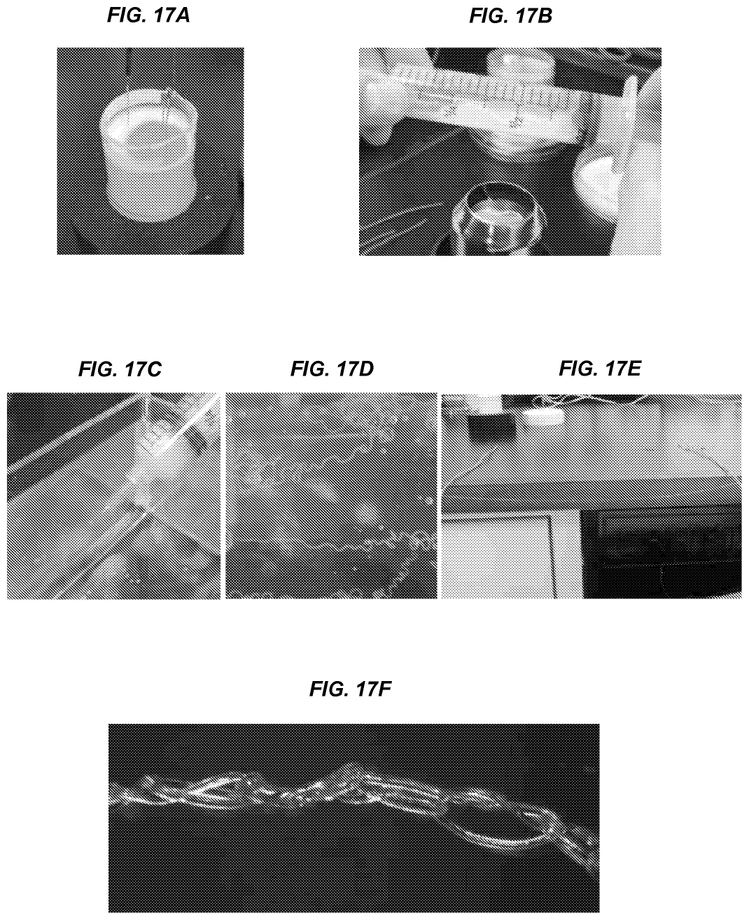

High molecular weight silk fibroin can be used to produce silk fibers with enhanced mechanical properties. Silk fibers have a variety of applications including, but not limited to, sutures and tissue engineering. FIG. 17F shows that a high molecular weight silk fibroin fiber is mechanically strong enough to form several knots.

Drug Delivery Devices:

In alternative embodiments, a drug delivery device (e.g., an implantable microchip or scaffold, or an injectable drug depot) or wound dressing (e.g., a bandage or an adhesive) can comprise a solid-state silk fibroin having high molecular weight silk fibroin encapsulated with at least one active agent therein. In some embodiments, a multi-layered silk fibroin structure can comprise at least one layer having high molecular weight silk fibroin encapsulated with at least one active agent therein.

Without limitations, high molecular weight silk fibroin can also be used in applications such as protective clothing, energy, immobilization of enzymes, cosmetics and affinity membranes (See, e.g., Bhardwaj, N. and S. C. Kundu, (2010) “Electrospinning: A fascinating fiber fabrication technique” Biotechnology Advances. 28(3): p. 325-347; Huang, Z.-M., et al., A review on polymer nanofibers by electrospinning and their applications in nanocomposites. Composites Science and Technology, 2003. 63(15): p. 2223-2253; Nisbet, D. R., et al., Review Paper: A Review of the Cellular Response on Electrospun Nanofibers for Tissue Engineering. Journal of Biomaterials Applications, 2009. 24(1): p. 7-29).

### Exemplary Active Agents

Active agent(s) can be introduced into the composition or solid-state silk fibroin described herein during or after its formation. For example, active agent(s) can be mixed into the silk fibroin solution prior to fabrication of the solid-state silk fibroin. Alternatively, the solid-state silk fibroin described herein can be fabricated and shaped into a desired shape, and then exposed to the active agent(s) in solution. As used herein, the term “active agent” refers to any molecule, compound or composition that is biologically active or has biological activity.

As used herein, the term “biological activity” refers to the ability of an agent to affect a biological sample. Biological activity can include, without limitation, elicitation of a stimulatory, inhibitory, regulatory, toxic or lethal response in a biological assay at the molecular, cellular, tissue or organ levels. For example, a biological activity can refer to the ability of a compound to exhibit or modulate the effect/activity of an enzyme, block a receptor, stimulate a receptor, modulate the expression level of one or more genes, modulate cell proliferation, modulate cell division, modulate cell morphology, modulate cell adhesion, modulate migration, or any combination thereof. In some instances, a biological activity can refer to the ability of a compound to produce a toxic effect in a biological sample, or it can refer to an ability to chemically modify a target molecule or cell.

At least one active agent (e.g., 1, 2, 3, 4, 5 or more active agents) can be included in the composition or solid-state silk fibroin described herein. Examples of active agent(s) include, without limitation, a therapeutic agent, or a biological material, such as cells (including stem cells such as induced pluripotent stem cells), proteins, peptides, nucleic acids (e.g., DNA, RNA, siRNA), nucleic acid analogs, nucleotides, oligonucleotides, peptide nucleic acids (PNA), aptamers, antibodies or fragments or portions thereof (e.g., paratopes or complementarity-determining regions), antigens or epitopes, hormones, hormone antagonists, growth factors or recombinant growth factors and fragments and variants thereof, cell attachment mediators (such as RGD), cytokines, enzymes, small molecules, antibiotics or antimicrobial compounds, viruses, antivirals, toxins, therapeutic agents and prodrugs, small molecules and any combinations thereof. See, e.g., WO 2009/140588; U.S. Patent Application Ser. No. 61/224,618). The active agent can also be a combination of any of the above-mentioned agents. Encapsulating either a therapeutic agent or biological material, or the combination of them, is desirous because the encapsulated composition can be used for numerous biomedical purposes.

In some embodiments, the active agent can also be an organism such as a fungus, plant, animal, bacterium, or a virus (including bacteriophage). Moreover, the active agent may include neurotransmitters, hormones, intracellular signal transduction agents, pharmaceutically active agents, toxic agents, agricultural chemicals, chemical toxins, biological toxins, microbes, and animal cells such as neurons, liver cells, and immune system cells. The active agents may also include therapeutic compounds, such as pharmacological materials, vitamins, sedatives, hypnotics, prostaglandins and radiopharmaceuticals.

Exemplary cells suitable for use herein may include, but are not limited to, progenitor cells or stem cells (including, e.g., induced pluripotent stem cells), smooth muscle cells, skeletal muscle cells, cardiac muscle cells, epithelial cells, endothelial cells, urothelial cells, fibroblasts, myoblasts, ocular cells, chondrocytes, chondroblasts, osteoblasts, osteoclasts, keratinocytes, kidney tubular cells, kidney basement membrane cells, integumentary cells, bone marrow cells, hepatocytes, bile duct cells, pancreatic islet cells, thyroid, parathyroid, adrenal, hypothalamic, pituitary, ovarian, testicular, salivary gland cells, adipocytes, and precursor cells. The active agents can also be the combinations of any of the cells listed above. See also WO 2008/106485; WO 2010/040129; WO 2007/103442.

As used herein, the terms “proteins” and “peptides” are used interchangeably herein to designate a series of amino acid residues connected to the other by peptide bonds between the alpha-amino and carboxy groups of adjacent residues. The terms “protein”, and “peptide”, which are used interchangeably herein, refer to a polymer of protein amino acids, including modified amino acids (e.g., phosphorylated, glycated, etc.) and amino acid analogs, regardless of its size or function. Although “protein” is often used in reference to relatively large polypeptides, and “peptide” is often used in reference to small polypeptides, usage of these terms in the art overlaps and varies. The term “peptide” as used herein refers to peptides, polypeptides, proteins and fragments of proteins, unless otherwise noted. The terms “protein” and “peptide” are used interchangeably herein when referring to a gene product and fragments thereof. Thus, exemplary peptides or proteins include gene products, naturally occurring proteins, homologs, orthologs, paralogs, fragments and other equivalents, variants, fragments, and analogs of the foregoing.

The term “nucleic acids” used herein refers to polynucleotides such as deoxyribonucleic acid (DNA), and, where appropriate, ribonucleic acid (RNA), polymers thereof in either single- or double-stranded form. Unless specifically limited, the term encompasses nucleic acids containing known analogs of natural nucleotides, which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer, et al., Nucleic Acid Res. 19:5081 (1991); Ohtsuka, et al., J. Biol. Chem. 260:2605-2608 (1985), and Rossolini, et al., Mol. Cell. Probes 8:91-98 (1994)). The term “nucleic acid” should also be understood to include, as equivalents, derivatives, variants and analogs of either RNA or DNA made from nucleotide analogs, and, single (sense or antisense) and double-stranded polynucleotides. The term “nucleic acid” also encompasses modified RNA (modRNA). The term “nucleic acid” also encompasses siRNA, shRNA, or any combinations thereof.

The term “modified RNA” means that at least a portion of the RNA has been modified, e.g., in its ribose unit, in its nitrogenous base, in its internucleoside linkage group, or any combinations thereof. Accordingly, in some embodiments, a “modified RNA” may contain a sugar moiety which differs from ribose, such as a ribose monomer where the 2′—OH group has been modified. Alternatively, or in addition to being modified at its ribose unit, a “modified RNA” may contain a nitrogenous base which differs from A, C, G and U (a “non-RNA nucleobase”), such as T or MeC. In some embodiments, a “modified RNA” may contain an internucleoside linkage group which is different from phosphate (—O—P(O)2-O—), such as —O—P(O,S)—O—. In some embodiments, a modified RNA can encompass locked nucleic acid (LNA).

The term “short interfering RNA” (siRNA), also referred to herein as “small interfering RNA” is defined as an agent which functions to inhibit expression of a target gene, e.g., by RNAi. An siRNA can be chemically synthesized, it can be produced by in vitro transcription, or it can be produced within a host cell. siRNA molecules can also be generated by cleavage of double stranded RNA, where one strand is identical to the message to be inactivated. The term “siRNA” refers to small inhibitory RNA duplexes that induce the RNA interference (RNAi) pathway. These molecules can vary in length (generally 18-30 base pairs) and contain varying degrees of complementarity to their target mRNA in the antisense strand. Some, but not all, siRNA have unpaired overhanging bases on the 5′ or 3′ end of the sense 60 strand and/or the antisense strand. The term “siRNA” includes duplexes of two separate strands, as well as single strands that can form hairpin structures comprising a duplex region.

The term “shRNA” as used herein refers to short hairpin RNA which functions as RNAi and/or siRNA species but differs in that shRNA species are double stranded hairpin-like structure for increased stability. The term “RNAi” as used herein refers to interfering RNA, or RNA interference molecules are nucleic acid molecules or analogues thereof for example RNA-based molecules that inhibit gene expression. RNAi refers to a means of selective post-transcriptional gene silencing. RNAi can result in the destruction of specific mRNA, or prevents the processing or translation of RNA, such as mRNA.

The term “enzymes” as used here refers to a protein molecule that catalyzes chemical reactions of other substances without it being destroyed or substantially altered upon completion of the reactions. The term can include naturally occurring enzymes and bioengineered enzymes or mixtures thereof. Examples of enzyme families include, but are not limited to, peroxidase, lipase, amylose, organophosphate dehydrogenase, ligases, restriction endonucleases, ribonucleases, DNA polymerases, glucose oxidase, laccase, kinases, dehydrogenases, oxidoreductases, GTPases, carboxyl transferases, acyl transferases, decarboxylases, transaminases, racemases, methyl transferases, formyl transferases, and α-ketodecarboxylases.

As used herein, the term “aptamers” means a single-stranded, partially single-stranded, partially double-stranded or double-stranded nucleotide sequence capable of specifically recognizing a selected non-oligonucleotide molecule or group of molecules. In some embodiments, the aptamer recognizes the non-oligonucleotide molecule or group of molecules by a mechanism other than Watson-Crick base pairing or triplex formation. Aptamers can include, without limitation, defined sequence segments and sequences comprising nucleotides, ribonucleotides, deoxyribonucleotides, nucleotide analogs, modified nucleotides and nucleotides comprising backbone modifications, branchpoints and nonnucleotide residues, groups or bridges. Methods for selecting aptamers for binding to a molecule are widely known in the art and easily accessible to one of ordinary skill in the art.

As used herein, the term “antibody” or “antibodies” refers to an intact immunoglobulin or to a monoclonal or polyclonal antigen-binding fragment with the Fc (crystallizable fragment) region or FcRn binding fragment of the Fc region. The term “antibodies” also includes “antibody-like molecules”, such as fragments of the antibodies, e.g., antigen-binding fragments. Antigen-binding fragments can be produced by recombinant DNA techniques or by enzymatic or chemical cleavage of intact antibodies. “Antigen-binding fragments” include, inter alia, Fab, Fab′, F(ab′)2, Fv, dAb, and complementarity determining region (CDR) fragments, single-chain antibodies (scFv), single domain antibodies, chimeric antibodies, diabodies, and polypeptides that contain at least a portion of an immunoglobulin that is sufficient to confer specific antigen binding to the polypeptide. Linear antibodies are also included for the purposes described herein. The terms Fab, Fc, pFc′, F(ab′) 2 and Fv are employed with standard immunological meanings (Klein, Immunology (John Wiley, New York, N.Y., 1982); Clark, W. R. (1986) The Experimental Foundations of Modern Immunology (Wiley & Sons, Inc., New York); and Roitt, I. (1991) Essential Immunology, 7th Ed., (Blackwell Scientific Publications, Oxford)). Antibodies or antigen-binding fragments specific for various antigens are available commercially from vendors such as R&D Systems, BD Biosciences, e-Biosciences and Miltenyi, or can be raised against these cell-surface markers by methods known to those skilled in the art.

Exemplary antibodies that may be incorporated in silk fibroin include, but are not limited to, abciximab, adalimumab, alemtuzumab, basiliximab, bevacizumab, cetuximab, certolizumab pegol, daclizumab, eculizumab, efalizumab, gemtuzumab, ibritumomab tiuxetan, infliximab, muromonab-CD3, natalizumab, ofatumumab omalizumab, palivizumab, panitumumab, ranibizumab, rituximab, tositumomab, trastuzumab, altumomab pentetate, arcitumomab, atlizumab, bectumomab, belimumab, besilesomab, biciromab, canakinumab, capromab pendetide, catumaxomab, denosumab, edrecolomab, efungumab, ertumaxomab, etaracizumab, fanolesomab, fontolizumab, gemtuzumab ozogamicin, golimumab, igovomab, imciromab, labetuzumab, mepolizumab, motavizumab, nimotuzumab, nofetumomab merpentan, oregovomab, pemtumomab, pertuzumab, rovelizumab, ruplizumab, sulesomab, tacatuzumab tetraxetan, tefibazumab, tocilizumab, ustekinumab, visilizumab, votumumab, zalutumumab, and zanolimumab. The active agents can also be the combinations of any of the antibodies listed above.

As used herein, the term “Complementarity Determining Regions” (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (i.e. about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop.

The expression “linear antibodies” refers to the antibodies described in Zapata et al., Protein Eng., 8(10):1057-1062 (1995). Briefly, these antibodies comprise a pair of tandem Fd segments (VH-CH1-VH-CH1) which, together with complementary light chain polypeptides, form a pair of antigen binding regions. Linear antibodies can be bispecific or monospecific.

The expression “single-chain Fv” or “scFv” antibody fragments, as used herein, is intended to mean antibody fragments that comprise the VH and VL domains of antibody, wherein these domains are present in a single polypeptide chain. Preferably, the Fv polypeptide further comprises a polypeptide linker between the VH and VL domains which enables the scFv to form the desired structure for antigen binding. (The Pharmacology of Monoclonal Antibodies, vol. 113, Rosenburg and Moore eds., Springer-Verlag, New York, pp. 269-315 (1994)).

The term “diabodies,” as used herein, refers to small antibody fragments with two antigen-binding sites, which fragments comprise a heavy-chain variable domain (VH) Connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. (EP 404,097; WO 93/11161; Hollinger et ah, Proc. Natl. Acad. Sd. USA, P0:6444-6448 (1993)).

As used herein, the term “small molecules” refers to natural or synthetic molecules including, but not limited to, peptides, peptidomimetics, amino acids, amino acid analogs, polynucleotides, polynucleotide analogs, aptamers, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “antibiotics” or “antimicrobial compound” is used herein to describe a compound or composition which decreases the viability of a microorganism, or which inhibits the growth or reproduction of a microorganism. As used in this disclosure, an antibiotic is further intended to include an antimicrobial, bacteriostatic, or bactericidal agent. Exemplary antibiotics can include, but are not limited to, actinomycin; aminoglycosides (e.g., neomycin, gentamicin, tobramycin); β-lactamase inhibitors (e.g., clavulanic acid, sulbactam); glycopeptides (e.g., vancomycin, teicoplanin, polymixin); ansamycins; bacitracin; carbacephem; carbapenems; cephalosporins (e.g., cefazolin, cefaclor, cefditoren, ceftobiprole, cefuroxime, cefotaxime, cefipeme, cefadroxil, cefoxitin, cefprozil, cefdinir); gramicidin; isoniazid; linezolid; macrolides (e.g., erythromycin, clarithromycin, azithromycin); mupirocin; penicillins (e.g., amoxicillin, ampicillin, cloxacillin, dicloxacillin, flucloxacillin, oxacillin, piperacillin); oxolinic acid; polypeptides (e.g., bacitracin, polymyxin B); quinolones (e.g., ciprofloxacin, nalidixic acid, enoxacin, gatifloxacin, levaquin, ofloxacin, etc.); sulfonamides (e.g., sulfasalazine, trimethoprim, trimethoprim-sulfamethoxazole (co-trimoxazole), sulfadiazine); tetracyclines (e.g., doxycyline, minocycline, tetracycline, etc.); monobactams such as aztreonam; chloramphenicol; lincomycin; clindamycin; ethambutol; mupirocin; metronidazole; pefloxacin; pyrazinamide; thiamphenicol; rifampicin; thiamphenicl; dapsone; clofazimine; quinupristin; metronidazole; linezolid; isoniazid; piracil; novobiocin; trimethoprim; fosfomycin; fusidic acid; or other topical antibiotics. Optionally, the antibiotic agents may also be antimicrobial peptides such as defensins, magainin and nisin; or lytic bacteriophage. The antibiotic agents can also be the combinations of any of the agents listed above. See also PCT/US2010/026190.

As used herein, the term “antigens” refers to a molecule or a portion of a molecule capable of being bound by a selective binding agent, such as an antibody, and additionally capable of being used in an animal to elicit the production of antibodies capable of binding to an epitope of that antigen. An antigen may have one or more epitopes. The term “antigen” can also refer to a molecule capable of being bound by an antibody or a T cell receptor (TCR) if presented by MHC molecules. The term “antigen”, as used herein, also encompasses T-cell epitopes. An antigen is additionally capable of being recognized by the immune system and/or being capable of inducing a humoral immune response and/or cellular immune response leading to the activation of B- and/or T-lymphocytes. This may, however, require that, at least in certain cases, the antigen contains or is linked to a Th cell epitope and is given in adjuvant. An antigen can have one or more epitopes (B- and T-epitopes). The specific reaction referred to above is meant to indicate that the antigen will preferably react, typically in a highly selective manner, with its corresponding antibody or TCR and not with the multitude of other antibodies or TCRs which may be evoked by other antigens. Antigens as used herein may also be mixtures of several individual antigens.

As used herein, the term “therapeutic agent” generally means a molecule, group of molecules, complex or substance administered to an organism for diagnostic, therapeutic, preventative medical, or veterinary purposes. As used herein, the term “therapeutic agent” includes a “drug” or a “vaccine.” This term include externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term can also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA), ribonucleic acid (RNA), modified DNA or RNA, or mixtures or combinations thereof, including, for example, DNA nanoplexes.

The term “therapeutic agent” also includes an agent that is capable of providing a local or systemic biological, physiological, or therapeutic effect in the biological system to which it is applied. For example, the therapeutic agent can act to control infection or inflammation, enhance cell growth and tissue regeneration, control tumor growth, act as an analgesic, promote anti-cell attachment, and enhance bone growth, among other functions. Other suitable therapeutic agents can include anti-viral agents, hormones, antibodies, or therapeutic proteins. Other therapeutic agents include prodrugs, which are agents that are not biologically active when administered but, upon administration to a subject are converted to biologically active agents through metabolism or some other mechanism. Additionally, a silk-based composition can contain combinations of two or more therapeutic agents.

In some embodiments, different types of therapeutic agents that can be encapsulated or dispersed in a silk fibroin-based material can include, but not limited to, proteins, peptides, antigens, immunogens, vaccines, antibodies or portions thereof, antibody-like molecules, enzymes, nucleic acids, modified RNA, siRNA, shRNA, aptamers, small molecules, antibiotics, and any combinations thereof.

Exemplary therapeutic agents include, but are not limited to, those found in Harrison's Principles of Internal Medicine, 13th Edition, Eds. T. R. Harrison et al. McGraw-Hill N.Y., NY; Physicians Desk Reference, 50th Edition, 1997, Oradell N.J., Medical Economics Co.; Pharmacological Basis of Therapeutics, 8th Edition, Goodman and Gilman, 1990; United States Pharmacopeia, The National Formulary, USP XII NF XVII, 1990, the complete contents of all of which are incorporated herein by reference.

Therapeutic agents include the herein disclosed categories and specific examples. It is not intended that the category be limited by the specific examples. Those of ordinary skill in the art will recognize also numerous other compounds that fall within the categories and that are useful according to the present disclosure. Examples include a radiosensitizer, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifingal agent, a vaccine, a protein, or a nucleic acid. In a further aspect, the pharmaceutically active agent can be coumarin, albumin, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetaminophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists such as clonidine; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; antiarrhythmic agents such as quinidine, lidocaine, tocainide hydrochloride, mexiletine hydrochloride, digoxin, verapamil hydrochloride, propafenone hydrochloride, flecainide acetate, procainamide hydrochloride, moricizine hydrochloride, and disopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as Coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hdyrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides.

Anti-cancer agents include alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists/antagonists, endothelinA receptor antagonists, retinoic acid receptor agonists, immuno-modulators, hormonal and antihormonal agents, photodynamic agents, and tyrosine kinase inhibitors.

Antibiotics include aminoglycosides (e.g., gentamicin, tobramycin, netilmicin, streptomycin, amikacin, neomycin), bacitracin, corbapenems (e.g., imipenem/cislastatin), cephalosporins, colistin, methenamine, monobactams (e.g., aztreonam), penicillins (e.g., penicillin G, penicillinV, methicillin, natcillin, oxacillin, cloxacillin, dicloxacillin, ampicillin, amoxicillin, carbenicillin, ticarcillin, piperacillin, mezlocillin, azlocillin), polymyxin B, quinolones, and vancomycin; and bacteriostatic agents such as chloramphenicol, clindanyan, macrolides (e.g., erythromycin, azithromycin, clarithromycin), lincomyan, nitrofurantoin, sulfonamides, tetracyclines (e.g., tetracycline, doxycycline, minocycline, demeclocyline), and trimethoprim. Also included are metronidazole, fluoroquinolones, and ritampin.

Enzyme inhibitors are substances which inhibit an enzymatic reaction. Examples of enzyme inhibitors include edrophonium chloride, N-methylphysostigmine, neostigmine bromide, physostigmine sulfate, tacrine, tacrine, 1-hydroxy maleate, iodotubercidin, p-bromotetramiisole, 10-(alpha-diethylaminopropionyl)-phenothiazine hydrochloride, calmidazolium chloride, hemicholinium-3,3,5-dinitrocatechol, diacylglycerol kinase inhibitor I, diacylglycerol kinase inhibitor II, 3-phenylpropargylamine, N′-monomethyl-Larginine acetate, carbidopa, 3-hydroxybenzylhydrazine, hydralazine, clorgyline, deprenyl, hydroxylamine, iproniazid phosphate, 6-Me0-tetrahydro-9H-pyrido-indole, nialamide, pargyline, quinacrine, semicarbazide, tranylcypromine, N,N-diethylaminoethyl-2,2-diphenylvalerate hydrochloride, 3-isobutyl-1-methylxanthne, papaverine, indomethacind, 2-cyclooctyl-2-hydroxyethylamine hydrochloride, 2,3-dichloro-a-methylbenzylamine (DCMB), 8,9-dichloro-2,3,4, 5-tetrahydro-1H-2-benzazepine hydrochloride, p-amino glutethimide, p-aminoglutethimide tartrate, 3-iodotyrosine, alpha-methyltyrosine, acetazolamide, dichlorphenamide, 6-hydroxy-2-benzothiazolesulfonamide, and allopurinol.

Antihistamines include pyrilamine, chlorpheniramine, and tetrahydrazoline, among others.

Anti-inflammatory agents include corticosteroids, nonsteroidal anti-inflammatory drugs (e.g., aspirin, phenylbutazone, indomethacin, sulindac, tolmetin, ibuprofen, piroxicam, and fenamates), acetaminophen, phenacetin, gold salts, chloroquine, D-Penicillamine, methotrexate colchicine, allopurinol, probenecid, and sulfinpyrazone.

Muscle relaxants include mephenesin, methocarbomal, cyclobenzaprine hydrochloride, trihexylphenidyl hydrochloride, levodopa/carbidopa, and biperiden.

Anti-spasmodics include atropine, scopolamine, oxyphenonium, and papaverine.

Analgesics include aspirin, phenybutazone, idomethacin, sulindac, tolmetic, ibuprofen, piroxicam, fenamates, acetaminophen, phenacetin, morphine sulfate, codeine sulfate, meperidine, nalorphine, opioids (e.g., codeine sulfate, fentanyl citrate, hydrocodone bitartrate, loperamide, morphine sulfate, noscapine, norcodeine, normorphine, thebaine, nor-binaltorphimine, buprenorphine, chlomaltrexamine, funaltrexamione, nalbuphine, nalorphine, naloxone, naloxonazine, naltrexone, and naltrindole), procaine, lidocain, tetracaine and dibucaine.

Ophthalmic agents include sodium fluorescein, rose bengal, methacholine, adrenaline, cocaine, atropine, alpha-chymotrypsin, hyaluronidase, betaxalol, pilocarpine, timolol, timolol salts, and combinations thereof.

Prostaglandins are art recognized and are a class of naturally occurring chemically related, long-chain hydroxy fatty acids that have a variety of biological effects.

Anti-depressants are substances capable of preventing or relieving depression. Examples of anti-depressants include imipramine, amitriptyline, nortriptyline, protriptyline, desipramine, amoxapine, doxepin, maprotiline, tranylcypromine, phenelzine, and isocarboxazide.

Trophic factors are factors whose continued presence improves the viability or longevity of a cell. Trophic factors include, Without limitation, platelet-derived growth factor (PDGP), neutrophil-activating protein, monocyte chemoattractant protein, macrophage-inflammatory protein, platelet factor, platelet basic protein, and melanoma growth stimulating activity; epidermal growth factor, transforming growth factor (alpha), fibroblast growth factor, platelet-derived endothelial cell growth factor, insulin-like growth factor, glial derived growth neurotrophic factor, ciliary neurotrophic factor, nerve growth factor, bone growth/cartilage-inducing factor (alpha and beta), bone morphogenetic proteins, interleukins (e.g., interleukin inhibitors or interleukin receptors, including interleukin 1 through interleukin 10), interferons (e.g., interferon alpha, beta and gamma), hematopoietic factors, including erythropoietin, granulocyte colony stimulating factor, macrophage colony stimulating factor and granulocyte-macrophage colony stimulating factor; tumor necrosis factors, and transforming growth factors (beta), including beta-1, beta-2, beta-3, inhibin, and activin.

Hormones include estrogens (e.g., estradiol, estrone, estriol, diethylstibestrol, quinestrol, chlorotrianisene, ethinyl estradiol, mestranol), anti-estrogens (e.g., clomiphene, tamoxifen), progestins (e.g., medroxyprogesterone, norethindrone, hydroxyprogesterone, norgestrel), antiprogestin (mifepristone), androgens (e.g, testosterone cypionate, fluoxymesterone, danazol, testolactone), anti-androgens (e.g., cyproterone acetate, flutamide), thyroid hormones (e.g., triiodothyronne, thyroxine, propylthiouracil, methimazole, and iodixode), and pituitary hormones (e.g., corticotropin, sumutotropin, oxytocin, and vasopressin). Hormones are commonly employed in hormone replacement therapy and/or for purposes of birth control. Steroid hormones, such as prednisone, are also used as immunosuppressants and anti-inflammatories.

Embodiments of various aspects described herein can be defined in any of the following numbered paragraphs:

One aspect provided herein is a composition comprising a solid-state silk fibroin, wherein the silk fibroin has an average molecular weight of at least about 200 kDa, and wherein no more than 30% of the silk fibroin has a molecular weight of less than 100 kDa.

In one embodiment of the composition, the solid-state silk fibroin can have a sericin content of less than 5%.

In some embodiments of the above-identified composition, the solid-state silk fibroin can be in a form selected from the group consisting of a film, a sheet, a gel or hydrogel, a mesh, a mat, a non-woven mat, a fabric, a scaffold, a tube, a slab or block, a fiber, a particle, powder, a 3-dimensional construct, an implant, a foam or a sponge, a needle, a lyophilized article, and any combinations thereof.

In some embodiments of the above-identified composition, the composition can further comprise an additive.

In one embodiment of the above-identified composition, the additive can be selected from the group consisting of biocompatible polymers; plasticizers; stimulus-responsive agents; small organic or inorganic molecules; saccharides; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; glycogens or other sugars; immunogens; antigens; an extract made from biological materials such as bacteria, plants, fungi, or animal cells; animal tissues; naturally occurring or synthetic compositions; and any combinations thereof.

In some embodiments of the above-identified composition, the additive can be in a form selected from the group consisting of a particle, a fiber, a tube, a film, a gel, a mesh, a mat, a non-woven mat, a powder, and any combinations thereof.

In one embodiment of the above-identified composition where the additive comprises a particle, the particle can be a nanoparticle or a microparticle.

In some embodiments of the above-identified composition, the additive can comprise a calcium phosphate (CaP) material, e.g., apatite.

In some embodiments of the above-identified composition, the additive can comprise a silk material, e.g., silk particles, silk fibers, micro-sized silk fibers, and unprocessed silk fibers.

In some embodiments of the above-identified composition, the composition can further comprise an active agent.

In one embodiment of the above-identified composition, the active agent can comprise a therapeutic agent.

In some embodiments of the above-identified composition, the composition can comprise from about 0.1% (w/w) to about 99% (w/w) of the additive agent and/or active agent.

Another aspect provided herein relates to an article comprising any one of the above-identified embodiments of the composition.

A further aspect provided herein is a silk fibroin article comprising silk fibroin at a mass concentration of no more than 2 grams of the silk fibroin per cubic centimeters of the silk fibroin article, and having an elastic modulus of at least about 0.15 kPa or an ultimate tensile strength of at least about 5 kPa.

In one embodiment of the above-identified silk fibroin article, at least about 70% of the silk fibroin can have a molecular weight of at least about 100 kDa.

In some embodiments of the above-identified silk fibroin article, the silk fibroin article can be in a form selected from the group consisting of a film, a sheet, a gel or hydrogel, a mesh, a mat, a non-woven mat, a fabric, a scaffold, a tube, a slab or block, a fiber, a particle, powder, a 3-dimensional construct, an implant, a foam or a sponge, a needle, a lyophilized article, and any combinations thereof.

Another aspect provided herein is a method of producing a silk fibroin article comprising: (i) providing a composition comprising silk fibroin having an average molecular weight of at least 200 kDa, and wherein no more than 30% of the silk fibroin has a molecular weight of less than 100 kDa; and (ii) forming the silk fibroin article from the composition.

Also provided herein is a method of producing a silk fibroin article comprising: (i) providing a composition comprising silk fibroin, wherein the silk fibroin is produced by degumming silk cocoons at a temperature in a range of about 60° C. to about 90° C.; and (ii) forming the silk fibroin article from the composition. In one embodiment, the silk cocoons can be degummed for at least about 30 minutes.

A further aspect provided herein is a method of producing a silk fibroin article comprising: (i) providing a composition comprising silk fibroin, wherein the silk fibroin is produced by degumming silk cocoons for no more than 15 minutes at a temperature of at least about 90° C.; and (ii) forming the silk fibroin article from the composition.

In some embodiments of various aspects of the above-identified methods, the silk fibroin article can be formed from the composition by a process selected from the group consisting of gel spinning, lyophilization, casting, molding, electrospinning, machining, wet-spinning, dry-spinning, milling, spraying, phase separation, template-assisted assembly, rolling, compaction, and any combinations thereof.

In some embodiments of various aspects of the above-identified methods, the composition can be a solution or powder.

In some embodiments of various aspects of the above-identified methods, the method can further comprise subjecting the silk fibroin article to a post-treatment.

In one embodiment of the above-identified method, the post-treatment can comprise steam drawing.

In one embodiment of the above-identified method, the post-treatment can induce a conformational change in the silk fibroin in the article. In some embodiments, inducing conformational change can comprise one or more of lyophilization, water annealing, water vapor annealing, alcohol immersion, sonication, shear stress, electrogelation, pH reduction, salt addition, air-drying, electrospinning, stretching, or any combination thereof.

In some embodiments of various aspects of the above-identified methods, the silk fibroin article can be in a form selected from the group consisting of a film, a sheet, a gel or hydrogel, a mesh, a mat, a non-woven mat, a fabric, a scaffold, a tube, a slab or block, a fiber, a particle, powder, a 3-dimensional construct, an implant, a foam or a sponge, a needle, a lyophilized article, and any combinations thereof.

In some embodiments of various aspects of the above-identified methods, the silk fibroin article can further comprise an additive. In some embodiments, the additive can be selected from the group consisting of biocompatible polymers; plasticizers; stimulus-responsive agents; small organic or inorganic molecules; saccharides; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; glycogens or other sugars; immunogens; antigens; an extract made from biological materials such as bacteria, plants, fungi, or animal cells; animal tissues; naturally occurring or synthetic compositions; and any combinations thereof. In some embodiments, the additive can be in a form selected from the group consisting of a particle, a fiber, a film, a gel, a tube, a mesh, a mat, a non-woven mat, a powder, and any combinations thereof. In some embodiments, the particle can be a nanoparticle or a microparticle. In some embodiments, the additive can comprise a calcium phosphate (CaP) material, e.g., apatite. In some embodiments, the additive can comprise a silk material, e.g., silk particles, silk fibers, micro-sized silk fibers, and unprocessed silk fibers.

In some embodiments of various aspects of the above-identified methods, the silk fibroin article can further comprise an active agent. In one embodiment, the active agent can comprise a therapeutic agent.

In some embodiments of various aspects of the above-identified methods, the composition can comprise from about 0.1% (w/w) to about 99% (w/w) of the additive and/or active agent.

A still another aspect provided herein is a method of substantially removing sericin from silk cocoons comprising: (i) degumming silk cocoons for less than 5 minutes at a temperature of at least about 90° C.; or (ii) degumming silk cocoons for at least about 30 minutes at a temperature in a range of about 60° C. to about 90° C.

A yet another aspect provided herein is a composition comprising silk fibroin, wherein the solution is substantially free of sericin, and wherein sericin is removed by (i) degumming silk cocoons for less than 5 minutes at a temperature of at least about 90° C.; or (ii) degumming silk cocoons for at least about 30 minutes at a temperature in a range of about 60° C. to about 90° C.

A method of making a tubular composition is also provided herein. The method comprises (i) providing an aqueous solution of silk fibroin, wherein the molecular weight of silk fibroin is selected for a pre-determined degradation rate of a tubular composition to be formed; (ii) forming a tubular structure from the aqueous solution of silk fibroin; (iii) drying the tubular structure; and (iv) removing said preparation from said rod, whereby a tube comprising silk fibroin is prepared.

In one embodiment of the above-identified method, the method can further comprise preparing the aqueous solution by a method comprising degumming cocoons for at least about 5 mins, at least about 10 mins, at least about 20 mins, at least about 30 mins, at least about 1 hour.

In some embodiments of the above-identified method, decreasing degumming time can yield higher average molecular weight of silk fibroin. Accordingly, lower concentrations of high molecular weight silk fibroin can be used to form the tubular composition. Without wishing to be bound by theory, using lower concentrations of high molecular weight silk fibroin can increase the degradation rate of the tubular composition as compared to lower molecular weight counterparts at higher concentrations.

In some embodiments of the above-identified method, the tubular structure can be formed by contacting a rod of a selected diameter with the aqueous solution of silk fibroin to coat said rod in silk fibroin.

In one embodiment of the above-identified method, the method can further comprising removing the dried tubular structure from the rod, thereby forming a tubular structure comprising silk fibroin.

In some embodiments of the above-identified method, the tubular composition can comprise an active agent described herein. In some embodiments, the active agent can comprise a therapeutic agent selected from the group consisting of a protein, a peptide, a nucleic acid, an aptamer, an antibody, a therapeutic agent, a small molecule, and any combinations thereof.

In some embodiments of the above-identified method, the tubular composition can have an inner lumen diameter of less than 6 mm.

In some embodiments of the above-identified method, the tubular composition can have an inner lumen diameter of 0.1 mm to 6 mm.

### Some Selected Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are essential to the invention, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise.

The term “a plurality of” as used herein refers to 2 or more, including, e.g., 3 or more, 4 or more, 5 or more, 6 or more, 7 or more, 8 or more, 9 or more, 10 or more, 20 or more, 30 or more, 40 or more, 50 or more, 100 or more, 500 or more, 1000 or more, 5000 or more, or 10000 or more.

Other than in the operating examples, or where otherwise indicated, all numbers expressing quantities of ingredients or reaction conditions used herein should be understood as modified in all instances by the term “about.” The term “about” when used in connection with percentages may mean±5% of the value being referred to. For example, about 100 means from 95 to 105.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The term “statistically significant” or “significantly” refers to statistical significance and generally means at least two standard deviation (2SD) away from a reference level. The term refers to statistical evidence that there is a difference. It is defined as the probability of making a decision to reject the null hypothesis when the null hypothesis is actually true.

As used interchangeably herein, the term “substantially” means a proportion of at least about 60%, or preferably at least about 70% or at least about 80%, or at least about 90%, at least about 95%, at least about 97% or at least about 99% or more, or any integer between 70% and 100%. In some embodiments, the term “substantially” means a proportion of at least about 90%, at least about 95%, at least about 98%, at least about 99% or more, or any integer between 90% and 100%. In some embodiments, the term “substantially” can include 100%.

As used herein, the phrase “silk fibroin-based material” refers to a material in which the silk fibroin constitutes at least about 10% of the total material, including at least about 20%, at least about 30%, at least about 40%, at least about 50%, at least about 60%, at least about 70%, at least about 80%, at least about 90%, at least about 95%, up to and including 100% or any percentages between about 30% and about 100%, of the total material. In certain embodiments, the silk fibroin-based material can be substantially formed from silk fibroin. In various embodiments, the silk fibroin-based material can be substantially formed from silk fibroin and at least one active agent. In some embodiments where the silk fibroin constitute less than 100% of the total material, the silk fibroin-based material can comprise a different material and/or component including, but not limited to, a metal, a synthetic polymer, e.g., but not limited to, poly(vinyl alcohol) and poly(vinyl pyrrolidone), a hydrogel, nylon, an electronic component, an optical component, an active agent, any additive described herein, and any combinations thereof.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated may be further modified to incorporate features shown in any of the other embodiments disclosed herein.

The disclosure is further illustrated by the following examples which should not be construed as limiting. The examples are illustrative only, and are not intended to limit, in any manner, any of the aspects described herein. The following examples do not in any way limit the invention.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### Example 1. Exemplary Materials and Methods Used for Generating a Composition Comprising High Molecular Weight Silk Fibroin Having an Average Molecular Weight of at Least about 200 kDa

Silk Fibroin Solution.

Silkworm Bombyx mori cocoons were degummed through a modified extraction process as described in Sofia S et al. (2001) Journal of Biomedical Materials Research; 54: 139-148. Provided herein is an exemplary protocol to produce a composition of high molecular weight silk fibroin.
Cut cocoons and remove the pupae, pupae skins and any other dirt from the inside of the cocoon;
Degum the cocoon pieces in a ˜0.02M boiling sodium carbonate (Na₂CO₃) solution using a degumming time of 15 minutes or less; or degum the cocoon pieces at a temperature of about 60° C. to about 90° C. in a ˜0.02M sodium carbonate (Na₂CO₃) solution for at least about 30 minutes or longer; (in some embodiments, the cocoon pieces can be degummed at a temperature of about 60° C. to about 90° C. in a ˜0.02M sodium carbonate (Na₂CO₃) solution for less than 30 minutes or shorter, if the presence of some sericin is not a concern for a specific application)
Rinse the degummed silk fibroin in water (e.g., Milli-Q water) at least thrice, for at least half an hour each time.
Air dry the rinsed silk fibroin.
Dissolve the silk fibroin in a 9.3 M lithium bromide solution (Sigma Aldrich, Mo., USA, ReagentPlus >99%) at 60° C. and dialyze against water (e.g., Milli-Q water), e.g., with Slide-a-Lyzer dialysis cassettes (Thermo Scientific, IL, USA, MWCO 3,500) for about 2 days, regularly changing the water, e.g., every 6 hours.
Centrifuge the resulting aqueous silk solution twice, at approximately 11,000 rpm, for 20 minutes each time.
The resulting aqueous high molecular weight silk fibroin solution has a concentration between 7% wt/vol and 9% wt/vol silk fibroin.
Store the silk fibroin solution in a cooler at 4° C.

Wray, et al. discussed the degradation of silk proteins during degumming, assessing molecular weights of solutions degummed from 5 to 60 minutes in 0.02 M Na₂CO₃ solutions at boiling conditions. The results showed a shift toward lower molecular weights as the boiling time was increased. However, there was not a concomitant change in the conformation of the proteins as measured with FTIR (Wray, L. S., et al., Journal of Biomedical Materials Research Part B: Applied Materials, 2011, 99B (1): 89-101). Yamada et al. also discussed differences in the resulting molecular weight distributions according to degumming conditions; however, they were unable to work with the silk fibroin solution without significant gelling of the fibroin polymer.

So far no one has reported the manufacturing of silk materials or articles based on high molecular weight silk fibroin, and thus no one has been able to tested their mechanical properties.

Sericin Content.

Sericin content of the solutions was determined by calculating the percentage mass loss during the degumming process and comparing it to the average 26.3% sericin for Japanese cocoons. Silk cocoons were weighed prior to degumming and following complete drying after removal of the sericin coating. All data represent n=6 for boiled conditions and n=3 for 70° C. conditions. Percent residual sericin was calculated by subtracting the percent mass loss from 26.3% and then divided by 26.3%.

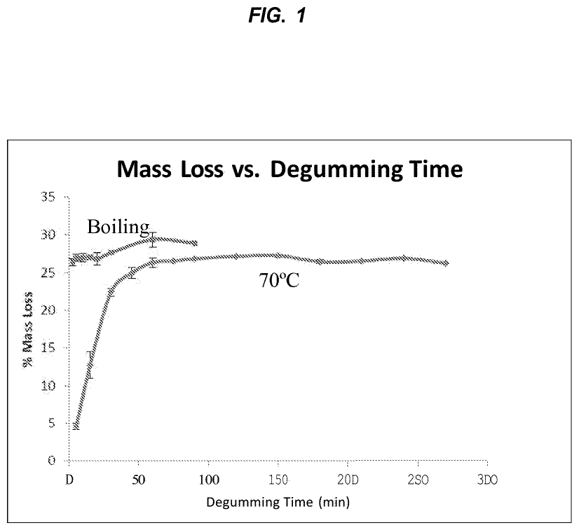

Effective removal of the sericin protein from the silk fibers is a fundamental step in preparing solution for use in vivo. The results of mass loss experiments indicated that sericin is substantially removed from the silk fibers after degumming for 2.5 minutes or less (e.g., less than 2 minutes or shorter) at a boiling temperature, or after 60 minutes when held at 70° C., as can be seen in FIG. 1. For the boiling condition there were no statistically significant differences (p>0.05) between the 2.5 mb, 5 mb, 10 mb, 15 mb, 20 mb and 30 mb groups. However, the 60 mb and 90 mb groups were significantly different (p<0.01) than all shorter degumming times, losing approximately 1% more mass than the other 6 conditions.

The 70° C. degumming in 0.02 M Na₂CO₃ solution resulted in almost complete sericin removal in approximately 60 minutes, with statistically significant additional mass loss (p<0.05) occurring at durations of 120 and 150 minutes. For both these groups an additional 0.5% of the initial fiber mass was lost during the degumming process. The 270 minute group exhibited a significant decrease in mass loss (p<0.05) as compared to the 90, 120, 150 and 240 minute groups. In addition to the verification of at least 26.3% loss of initial mass, the percent residual sericin was calculated for the 70° C.—5, 15, 30 and 45 minute groups as shown in Table 1. These calculations indicate that the amount of sericin removed is roughly proportional to the amount of time it is exposed to the 70° C., sodium carbonate degumming solution.

TABLE 1
Residual sericin content for degumming
in 70° C. sodium carbonate solution

| Degumming time at 70° C. | % Mass Loss | % Residual |
|---|---|---|
| 5 | 4.6 | 82.5 |
| 15 | 12.8 | 51.5 |
| 30 | 22.4 | 14.7 |
| 45 | 25.0 | 5.1 |
| 60 | 26.3 | 0.0 |

Gel Electrophoresis.

Gel electrophoresis is used to determine the molecular weight distribution of silk fibroin. The electrophoretic mobility of the fibroin molecules was determined using sodium dodecyl sulfate polyacrylamide gel electrophoresis (SDS-PAGE). For each condition of interest, 5 μg of silk protein was reduced and loaded into a 3-8% Tris Acetate gel (NuPAGE, Life Technologies, Grand Island, N.Y.). The gel was run under reducing conditions for 45 minutes at 200V, with a high molecular weight ladder as a reference (HiMark Unstained, Life Technologies) and stained with a Colloidal Blue staining kit (Life Technologies). The molecular weight distribution of the silk solutions was determined by imaging the gels, performing pixel density analysis and normalizing across all the lanes for a peak intensity value of one (ImageJ, NIH, Bethesda, Md.).

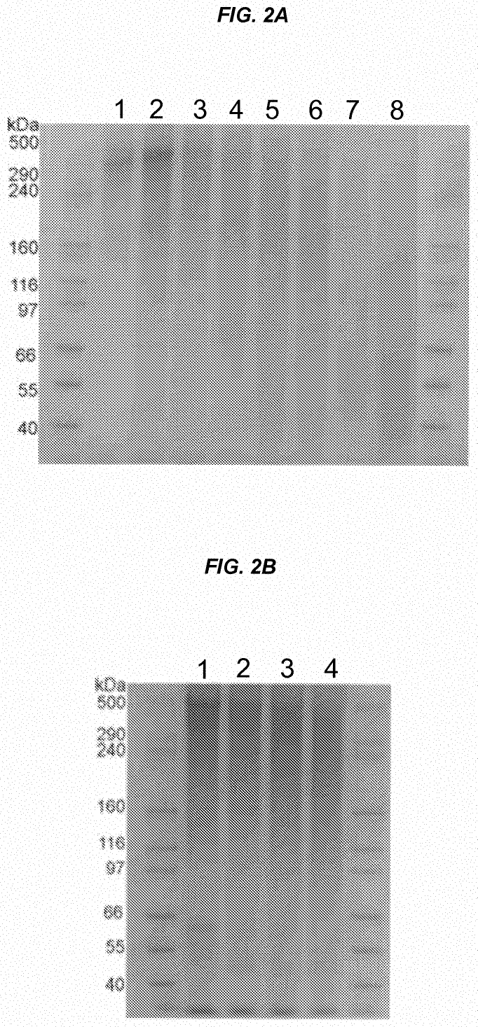

Increased degumming times and temperatures of the silk fibers correlated directly with a decrease in the average molecular weight of the proteins and resulted in a downward shift of the smear exhibited on the SDS-PAGE gels. This degradation with longer degumming times is clearly shown for both boiling temperatures and 70° C. degumming conditions in FIGS. 2A and 2B respectively.

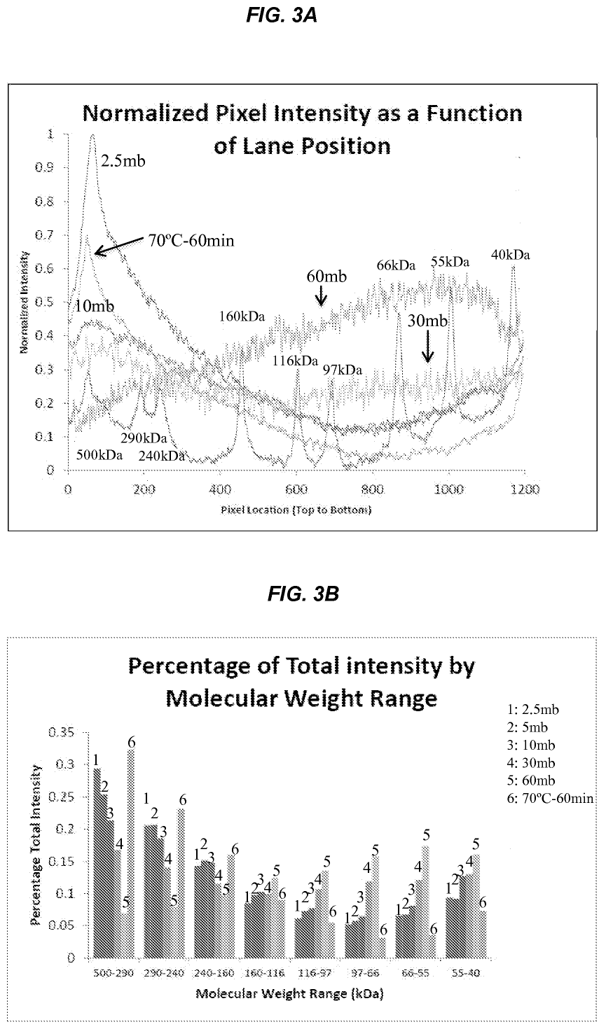

In addition to qualitative visual analysis, densiometric image analysis was performed on the electrophoresis gels. The raw pixel intensity for each was collected and the intensity values were normalized across all data groups to provide an intensity range from 0 to 1. Following normalization a wide range of groups were plotted against their lane position as shown in FIG. 3A. This clearly shows that the bulk of the proteins in low degumming conditions including 2.5 mb, 5 mb, 70 C-60 m and 70 C-90 m (not all data shown) are in the high molecular weight bands at approximately 500 kDa. At 10 min of degumming the pronounced peak at about 500 kDa has been eroded and the molecular weight distribution becomes more distributed between about 500 kDa to 100 kDa. At 30 minutes of boiling the protein is degraded to where its distribution is nearly equal across the whole range of weights visualized by the gel, including down to the 40 kDa range. Degumming for 60 minutes resulted in a pronounced shift in the molecular weight distribution of the silk solution, with a peak concentration occurring at approximately 60 kDa. The relative degradation profile of the silk solutions degummed at 70° C. is similar to that for boiled solutions, however, the kinetics is significantly retarded. This is clearly indicated by the similar characteristics of the 70 C-60 m group with the 2.5 mb group in FIG. 3A.

As shown in FIG. 3B, it presents the densiometric analysis while also accounting for differences in protein loading between the lanes. This plot shows the average contribution of the protein density between marker peaks as a function of the total protein loading for the lane. This analysis further clarifies the substantial impact on the molecular weight from additional degumming times. In addition, it suggests that the 70° C. degumming temperature may result in less degradation to the protein molecule as there is less of a contribution to the overall loading from bands below 160 kDa.

Viscosity measurements. Silk solutions were diluted to a concentration of 5% w/v, gently mixed and allowed to equilibrate overnight at 4° C. The following day the solutions were slowly brought to room temperature (25° C.) and dynamic viscosity of the solutions was tested using an RVDV-II+ cone and plate viscometer (Brookfield Engineering, Middleboro, Mass.). For solutions with a plastic viscosity above 20 cP, testing was done using a CP-52 cone, with a 1.2 cm cone radius and 3° cone angle over a shear rate range from 10-300 l/s. Solutions with a plastic viscosity below 20 cP were tested using CP-40 cone, with a 2.4 cm radius and 0.8° cone angle over a shear rate range of 37.5-1500 l/s. Following collection of the shear rate and torque, the data were analyzed and fitted using the Bingham Plastic model using Rheocalc V3.3 software (Brookfield Engineering). The Bingham plastic model assumes a Newtonian fluid behavior after an initial yield stress is overcome. The data is fitted to equation τ=τ⁰+ηD, where τ is the measured shear stress, τ^{o} is the yield stress, 11 is the plastic viscosity and D is the shear rate. During the analysis procedure the first two data points of each sample were removed to allow for full engagement of the sample with the cone. In addition, samples that exhibited signs of gelation, a rapid increase in shear stress, were eliminated and the tests repeated. Data represents three samples from three separate batches of silk solution.

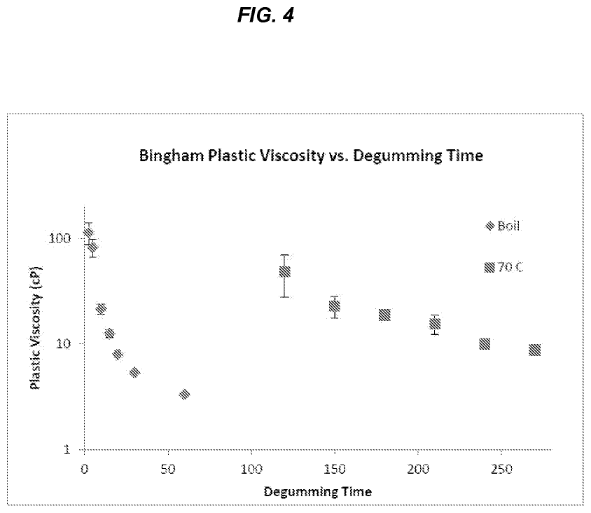

The plastic viscosities of solutions produced from a wide range of degumming conditions were characterized as shown in FIG. 4. The viscosities exhibited a roughly exponential behavior with a rapid decrease from a maximal plastic viscosity of 113 cP for 2.5 mb solution to a low of 3.3 cP for 60 mb solution. The same behavior was seen with the 70° C. solutions with a plastic viscosity of 48 cP for 70 C-120 m solution to 8.77 cP for 70 C-270 m solution. Note that viscosities were not collected for the 70 C-60 m and 70 C-90 m groups as there was a propensity for the solutions to gel upon the application of any shear which prevented consistent data collection.

Rheometry. Rheological measurements were taken using an ARES strain-controlled rheometer (TA Instruments, New Castle, Del.). Dynamic oscillatory frequency sweeps were taken using a 50 mm parallel plate geometry at room temperature (25° C.). The silk solution was loaded onto the bottom platen in a manner as to minimize shear and the upper platen was lowered to a gap distance of 0.5 mm with a maximum applied normal force of 0.05 N. The viscoelastic response of the silk solution was recorded with a strain magnitude of 1% and a wide range of frequencies from 0.1-100 rad/s. All solutions were at a concentration of 7.5% and were tested within 3 days of being removed from dialysis.

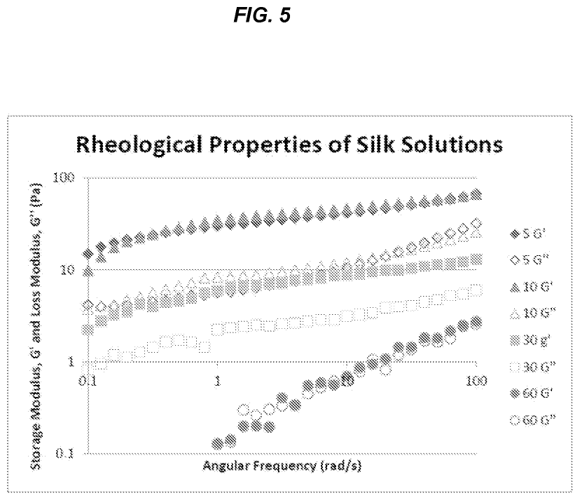

Full rheological behaviors of solutions were collected over a wide range of degumming conditions. As shown in FIG. 5, the shear and loss moduli for 5 mb, 10 mb, 30 mb and 60 mb cover a range of three orders of magnitude from 0.1 to 100 Pa and indicate a storage modulus greater than the loss magnitude. This indicates that the solutions are acting more like a “solid” or “elastic” material than that of a viscous liquid. The only sample that does not exhibit this behavior is the 60 mb group; however, the torque values are below the minimum range of the instrument and are of suspect validity. In addition, it is interesting to note that the 5 mb and 10 mb groups show similar behaviors and magnitudes despite the fact that the 10 mb sample was exposed to twice the degradation time.

Molecular weight analysis and viscosity data confirm that as degumming time and temperature are increased the fibroin proteins are subjected to greater degradation. While the kinetics of degradation is significantly slower at 70° C. versus boiling, the general trends are consistent with the sharp band near the 500 kDa marker at low degumming times slowly spreading and shifting downward as immersion times increased.

One potential concern with the SDS-PAGE gels is that the gently degummed silk has an apparent molecular weight that is on the order of 150 kDa higher than the generally accepted 350-370 kDa for native fibroin (Yeo, J. H., et al., Biological and Pharmaceutical Bulletin, 2000, 23(10): 1220-1223; Sasaki, T. and Noda, H., Biochimica et Biophysica Acta-Protein Structure, 1973, 310(1): 76-90). In order to allay these concerns, silk dope extracted from the B. mori silkworm was tested using the same protocol and the distinct fibroin and sericin bands were shifted up by the same 150 kDa (data not shown). Without wishing to be bound by theory, this discrepancy is likely due to differences in protein folding between the marker protein and silk fibroin as electrophoretic mobility is influenced by both protein folding and molecular weight.

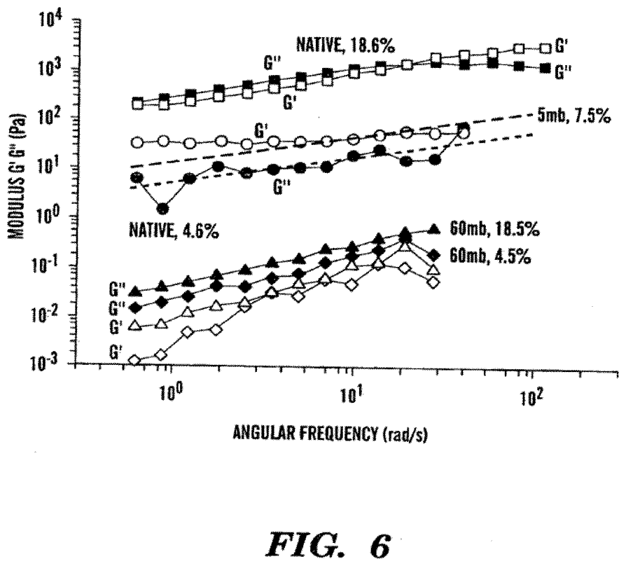

As shown in FIG. 6, where the rheological data from a 5 mb, 7.5% w/v solution are superimposed on Holland et al.'s data (Holland, C. et al., Polymer, 2007, 48(12):3388-3392), the 5 mb solution, while of slightly higher concentration, 7.5% w/v, than Holland et al.'s, 4.6%, displays the same behavior and modulus the low concentration native dope. In addition to their analogous moduli, the native 4.6% and regenerated 5 mb, 7.5% solutions exhibit the same behavior. Namely, the G′ and G″ values are inverted, suggesting a gel like state, instead of the viscous fluid expected. This inversion of properties is likely related to entanglements between unfolded protein chains.

### Example 2. Exemplary Methods Used for Making Silk Films and the Use Thereof

Silk films were casted at room temperature (about 25° C.) and a relative humidity of 15%-30% in a 100 mm polystyrene petri dish. Based on the solution concentration, an appropriate volume of silk solution to generate a 75 μm thick film, was gently poured into the petri dish, spread to achieve proper dispersion and any air bubbles removed. The films were allowed to dry for 24 hours before handling to ensure complete self-assembly and water evacuation and stored at room temperature and humidity. All solutions were casted within 10 days of their generation. As shown in FIGS. 7A-7B, the silk fibroin solution with short degumming time can be used to produce very large, high-quality films that are both strong and tough. In addition, the films can be formed on a diffraction pattern (FIG. 7B), suggesting the ability to embed small features on the surface of the time. The surprising toughness of the films give them a “plastic-like” feel, allowing the films to be handled and even rolled into a tight spiral. The traditional 30 minute or greater degumming time typically produces a film that is considerable more challenging to handle without film failure and has typically limited the size of the films to 2″×2″.

Post-treatments were performed on select films to determine inter-group differences in treatment response. Films from 5 mb, 15 mb, 30 mb and 60 mb groups were treated in either methanol or water annealed to induce transition to β-sheet. Methanol treated films were cut into 6.2 mm wide strips and soaked in 100% methanol at room temperature for 4 hours. The film strips were then removed from the methanol and placed in a hood and allowed to dry overnight to allow evaporation of residual methanol. Water annealed films were cut into 6.2 mm wide strips and placed in an evacuated bell-jar container with water in the bottom, at 37° C. for 2 hours. The films were subsequently removed and allowed to dry overnight in a hood.

Film Drawing.

One of the properties of silk that makes it useful for numerous applications is its overall toughness, or its ability to absorb energy without failure. This property is directly related to the fibroins extensibility. However, reconstituted silks are typically brittle under ambient, dry conditions. In order to improve the functionality of regenerated silks in their dry state, the extensibility of the materials needs to be increased toward that of the native silk fiber. Recent efforts have shown that the best method for improving silk film or fiber extensibility is to draw the specimen in the presence of a plasticizer after it has been formed.

Controlled drawing of rehydrated silk films produced by low molecular weight silk fibroin after casting and ethanol treatment was shown to improve tensile strength, elastic modulus, extensibility and tenacity by Yin, et al. (Yin, J., et al., Biomacromolecules, 2010, 11 (11): 2890-2895). Specifically silk films of 200 μm thickness, were casted from solutions that had been degummed twice for 30 minutes each, were rendered insoluble with ethanol treatment and allowed to fully rehydrate for 30 minutes in distilled water. The films were then stretched to 2 or 3 times their original lengths, allowed to dry and subjected to tensile testing. The results suggest that molecular alignment is critical to produce mechanical properties similar to those of native silk fibers.

Extensibility was increased after drawing, but not the modulus or tensile strengths, in films of low β-sheet content as reported by Lu, et al. Instead of inducing β-sheet via post-treatment with ethanol to generate insoluble films, the drying kinetics were retarded during casting, which results in films with higher a content that are also insoluble in water. These films were then hydrated for 30 minutes and stretched to 200% of their initial length. Zhang, C., et al. discusses that extensibility was increased by a factor of 10, while modulus and strength were halved (Zhang, C., et al., Biomacromolecules, 2012, 13 (7): 2148-2153).

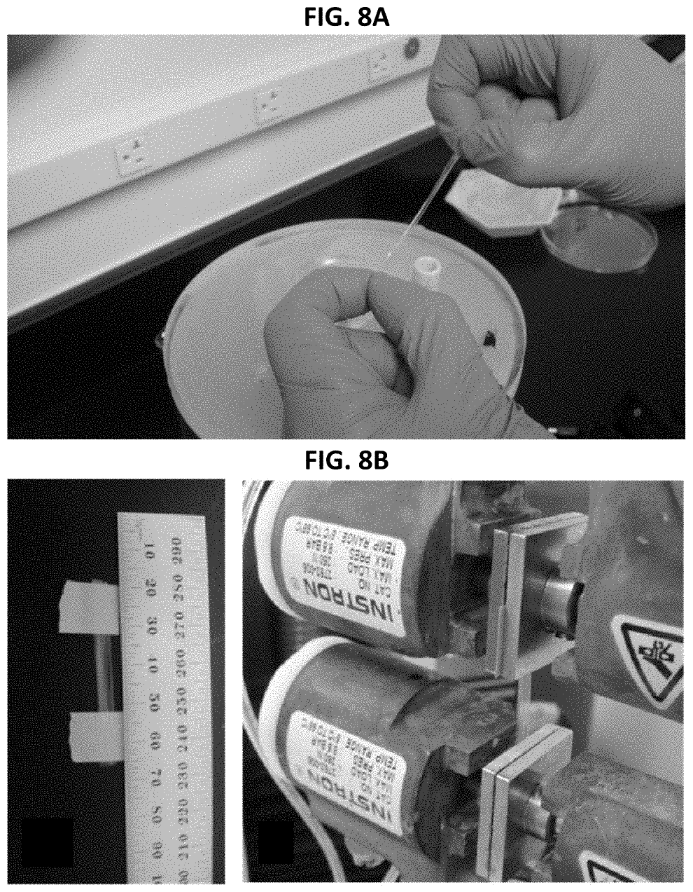

To evaluate drawing on silk films produced by high molecular weight silk fibroin, films were steam drawn in order to induce alignment of the molecules. Steam was chosen as the preferred plasticizer for drawing as it does not necessitate the film to be water insoluble. Insoluble films require treatment with methanol or water annealing which locks in the structure of material. By avoiding this step we increase the mobility of the molecules and should allow for a greater degree of workability and increased molecular alignment. After casting and drying, films were cut into 6.2 mm wide strips. These strips were hand drawn over a steam jet, as shown in FIG. 8A. Drawing commenced at one end of the film and proceeded along its length as the area exposed to steam reached its maximum extension. Maximum extension was determined when the application of additional tensile force or steam exposure would lead to film failure as tested in a screening strip. The distance between the grip locations was measured to the nearest millimeter before and after drawing. The draw ratio is obtained by dividing the overall length change by the initial length.

Tensile Testing.

All tensile testing was performed as previously described (Lu, Q. et al., Acta Biomaterialia, 2010, 6(4): 1380-1387). Specifically, a sample of 20 mm gage length was tested at a crosshead speed of 1.2 mm/min (0.1% strain/sec) and a preload of 0.5 N, using an Instron 3366 testing frame (Instron, Norwood, Mass.), with 100 N load cell. To prevent slippage or failure due to stress concentration at grip edges, specimens were prepared by applying a piece of doubled over tape at each grip location. Samples were then measured for length and width, values recorded and the sample mounted in the test fixture as shown in FIG. 8B. The specimens were tested until failure and load and extension data collected. All testing was performed at ambient temperature and humidity.

Tensile data were analyzed for linear elastic modulus, extensibility and ultimate tensile stress using a custom LabVIEW program. The modulus was calculated as the least squares fit between 1.5 to 3.5% strain. The extensibility was the strain achieved before a >10% decrease in applied load and the ultimate tensile stress was taken as the maximum engineering stress achieved throughout the test.

Conformational differences in the silk films were analyzed using a JASCO FTIR 6200 spectrometer (JASCO, Tokyo, Japan) with a MIRacle™ attenuated total reflection (ATR) Ge crystal cell in reflection mode. Silk films were tested fully dried and at ambient conditions. Background spectra were taken and subtracted from all sample readings. Each measurement represents the average of 64 scans taken at a resolution of 4 cm⁻¹ and wavenumbers ranging from 500 to 4000 cm⁻¹. Data were truncated to only include the amide I band from 1595 to 1705 cm⁻¹ and peak normalized.

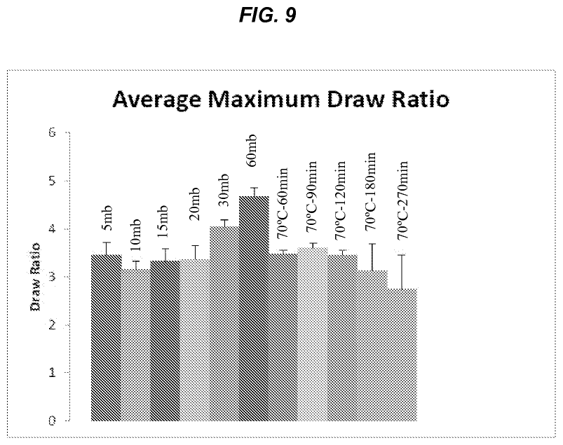

Steam drawing of the films resulted in a consistent draw ratio of 3.2-3.4 times the initial film length, regardless of degumming conditions, as indicated in FIG. 9. The only exception to this was in the samples from the 30 mb and 60 mb groups which exhibited significantly greater (p<0.01) draw ratios of 4 and 4.7 respectively.

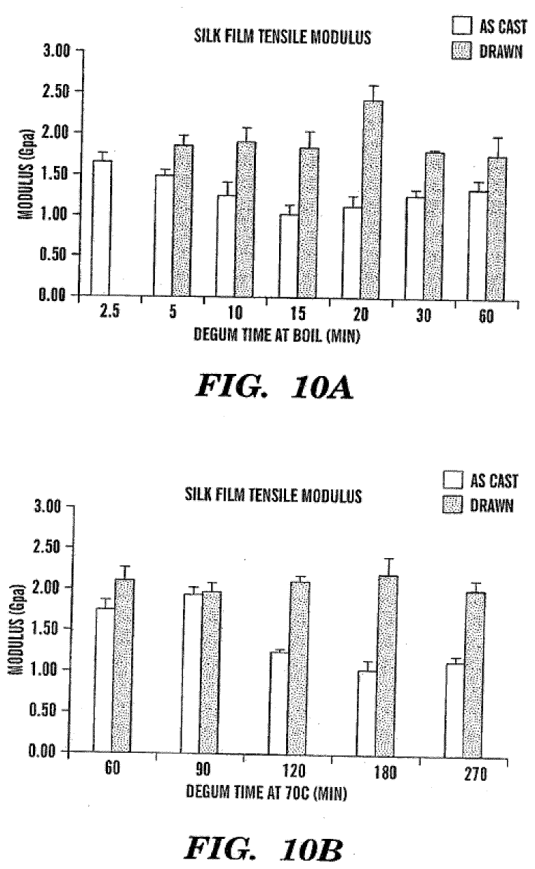

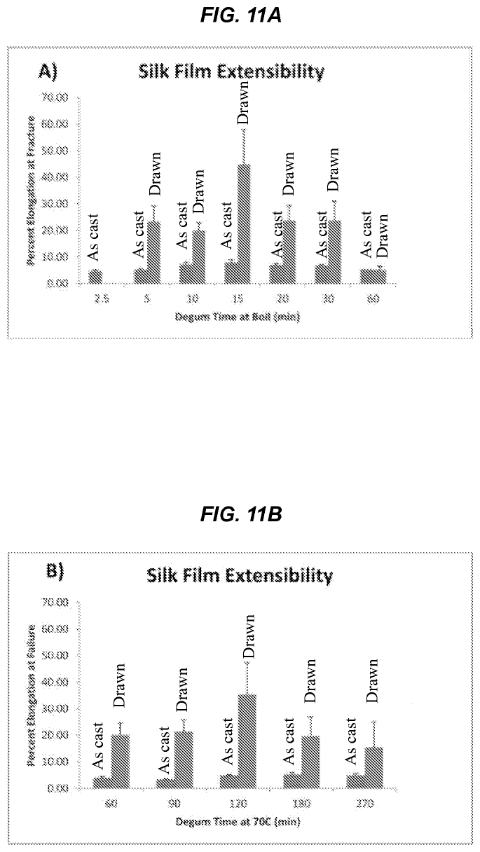

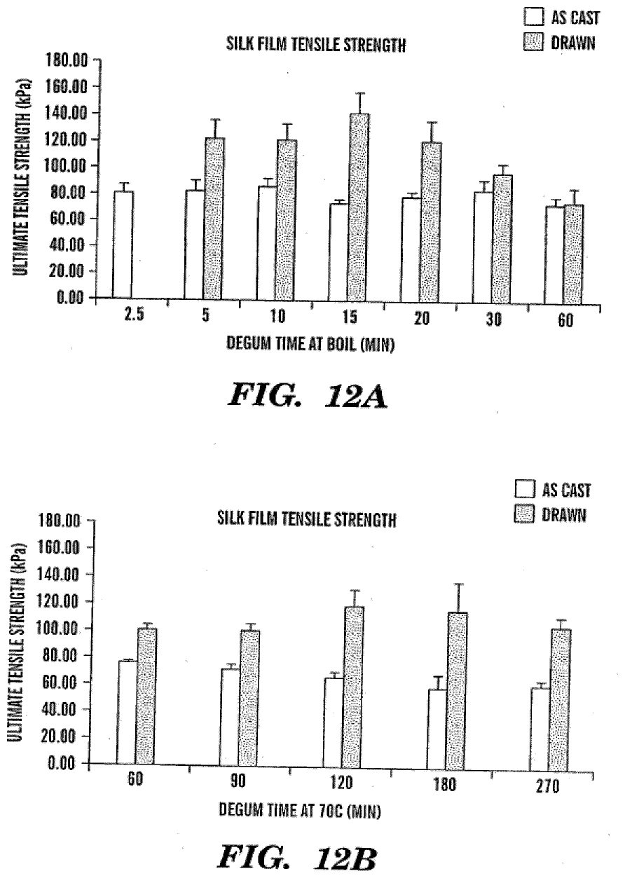

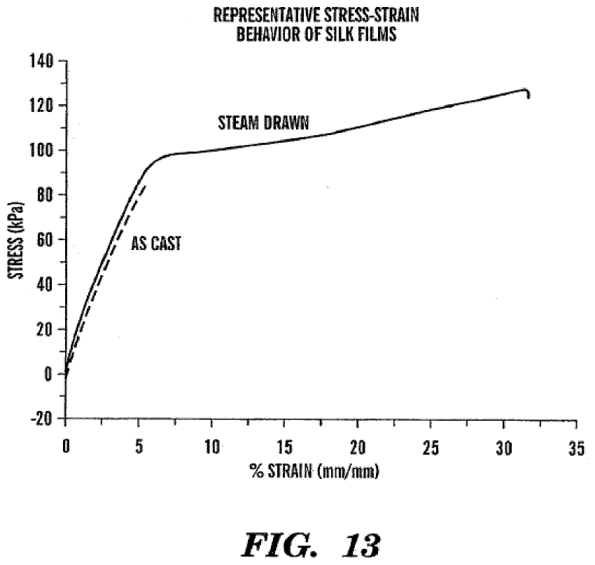

The linear elastic modulus, extensibility and ultimate tensile strength of differently degummed films in as cast and steam drawn conditions are shown in FIGS. 10, 111 and 12, respectively. Tabulated values of averages and standard deviation are also provided in Table 2. Representative stress-strain curves for as cast and steam drawn samples are shown in FIG. 13. In general, all as cast film samples, regardless of degumming conditions, exhibited a purely brittle behavior, with no distinct yield point and failure within the linear elastic region. The steam drawn samples, with the exception of the 60 mb group, showed behavior more typical of a ductile material, with a prominent yield and subsequent work hardening behavior until failure. In addition to overall material behavior changes with steam drawing, the stretching resulted in higher elastic moduli, extensibility and ultimate strengths for all experimental groups except the 70 C-90 m and 60 mb groups. The steam drawn 70 C-90 m group only showed increased extensibility and tensile strength while the 60 mb group only had increased modulus after drawing. The as cast modulus and steam drawn extensibility were also inversely related with the 15 mb and 70 C-120 m groups having moduli of approximately 1 GPa with extensibilities of 40% or greater after drawing.

TABLE 2
Tabulated mechanical data for as cast and steam drawn films
for boiled and 70° C. degummed samples

| | | | | Steam | | Steam |
|---|---|---|---|---|---|---|
| | | Steam | As Cast | Drawn | As Cast | Drawn |
| | As Cast | Drawn | Exten- | Exten- | Tensile | Tensile |
| | Modulus | Modulus | sibility | sibility | Strength | Strength |
| 5 mb | 1.5 ± 0.1 | 1.9 ± 0.1 | 5.3 ± 0.4 | 23.4 ± 5.8 | 83.3 ± 7.5 | 122.8 ± 13.4 |
| 10 mb | 1.3 ± 0.2 | 1.9 ± 0.2 | 7.2 ± 0.8 | 20.0 ± 2.8 | 86.5 ± 6.1 | 122.5 ± 11.2 |
| 15 mb | 1.0 ± 0.1 | 1.8 ± 0.2 | 7.8 ± 1.2 | 44.9 ± 13.1 | 74.3 ± 2.7 | 142.5 ± 15.2 |
| 20 mb | 1.1 ± 0.1 | 2.4 ± 0.2 | 7.1 ± 0.4 | 23.7 ± 2.8 | 79.1 ± 4.0 | 121.0 ± 14.9 |
| 30 mb | 1.3 ± 0.1 | 1.8 ± 0.0 | 6.8 ± 0.3 | 23.8 ± 7.2 | 84.7 ± 7.4 | 98.0 ± 6.6 |
| 60 mb | 1.3 ± 0.1 | 1.8 ± 0.2 | 5.4 ± 0.5 | 5.1 ± 1.3 | 74.3 ± 5.5 | 75.5 ± 11.2 |
| 70 C.- | 1.8 ± 0.1 | 2.1 ± 0.2 | 4.1 ± 0.3 | 20.2 ± 4.5 | 76.3 ± 1.2 | 101.0 ± 3.9 |
| 60 m | | | | | | |
| 70 C.- | 2.0 ± 0.1 | 2.0 ± 0.1 | 3.4 ± 0.3 | 21.4 ± 4.5 | 71.7 ± 3.5 | 100.3 ± 5.0 |
| 90 m | | | | | | |
| 70 C.- | 1.3 ± 0.0 | 2.1 ± 0.1 | 5.0 ± 0.2 | 35.4 ± 12.0 | 66.5 ± 3.6 | 119.9 ± 11.6 |
| 120 m | | | | | | |
| 70 C.- | 1.0 ± 0.1 | 2.2 ± 0.2 | 5.3 ± 0.7 | 19.7 ± 7.2 | 59.9 ± 8.8 | 117.4 ± 20.0 |
| 180 m | | | | | | |
| 70 C.- | 1.1 ± 0.1 | 2.0 ± 0.1 | 5.0 ± 0.5 | 15.6 ± 9.5 | 61.9 ± 4.0 | 105.4 ± 7.3 |
| 270 m | | | | | | |

FTIR Spectra.

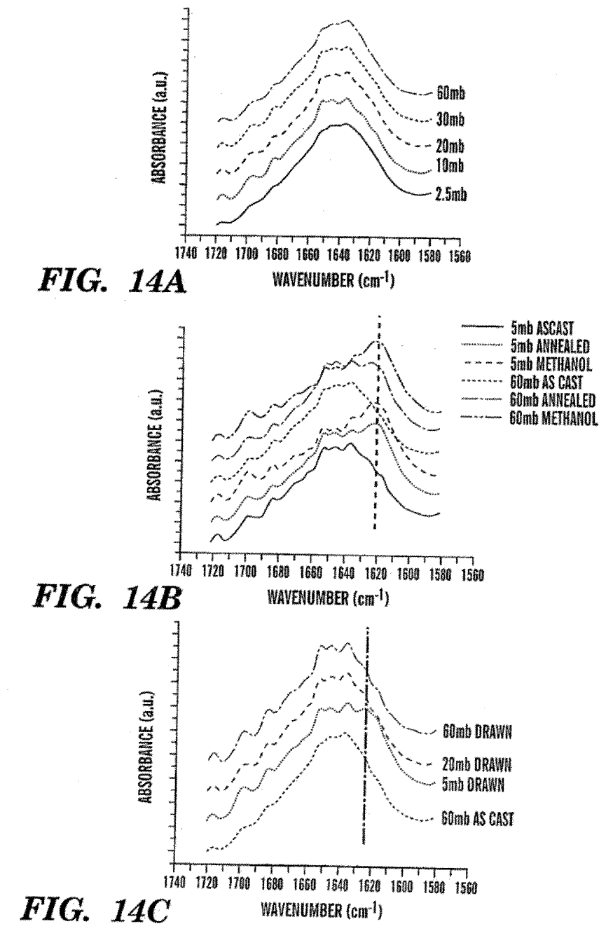

Comparison of the FTIR spectra of films casted from differently degummed solutions did not reveal any between-group differences in the as cast, post-treated, or steam drawn conditions as shown in FIGS. 14A-14C respectively. As cast samples all exhibited a primarily silk I conformation with a broad peak between 1650 and 1630 cm⁻¹. The conformational response to post-treatment was similarly not influenced by the molecular weight distribution of the fibroin. The spectral shifts toward 1620 cm⁻¹ exhibited in 5 mb, 30 mb (not shown) and 60 mb were characteristic of the transition to β-sheet in response to methanol treatment and water annealing. However, there were no differences in the response between experimental conditions. Additionally, the spectral shifts exhibited due to steam drawing did not indicate differences in conformation between the groups.

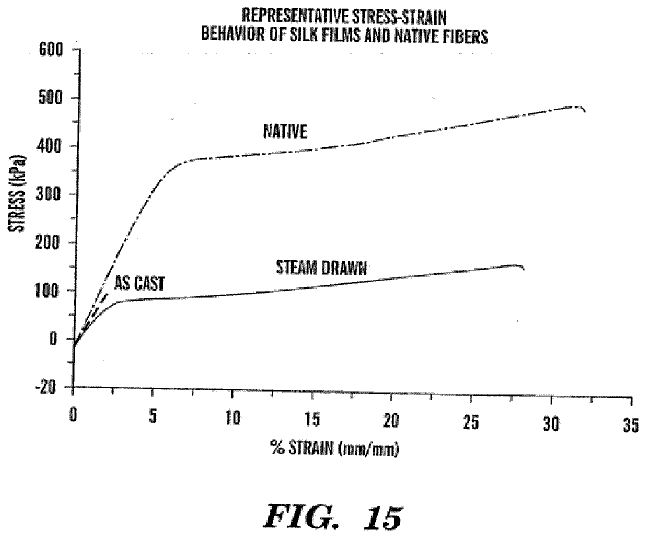

While gentle degumming conditions were able to generate silk solutions with similar rheological properties to native dope, they were unable to generate films to match the robust mechanical properties of the silk fibers. As depicted in FIG. 15, the best steam stretched films were able to roughly match the modulus and extensibility of native fibers, however, the breaking strength of the films was below 30% of the strength of fibers. In addition, the FTIR spectra of films from differently degummed silks respond similarly to post treatment steps, indicating that degumming does not significantly affect the conformation of the proteins.

Without wishing to be bound by theory, we propose mechanisms for three types of regenerated solutions, gently degummed (2.5 mb or 5 mb), standard degummed (15 mb or 20 mb) and aggressively degummed (60+mb).

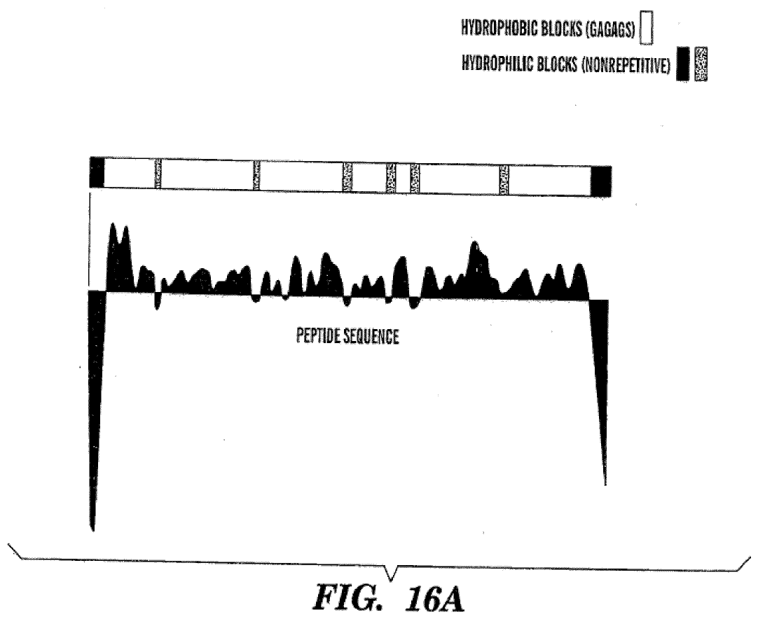

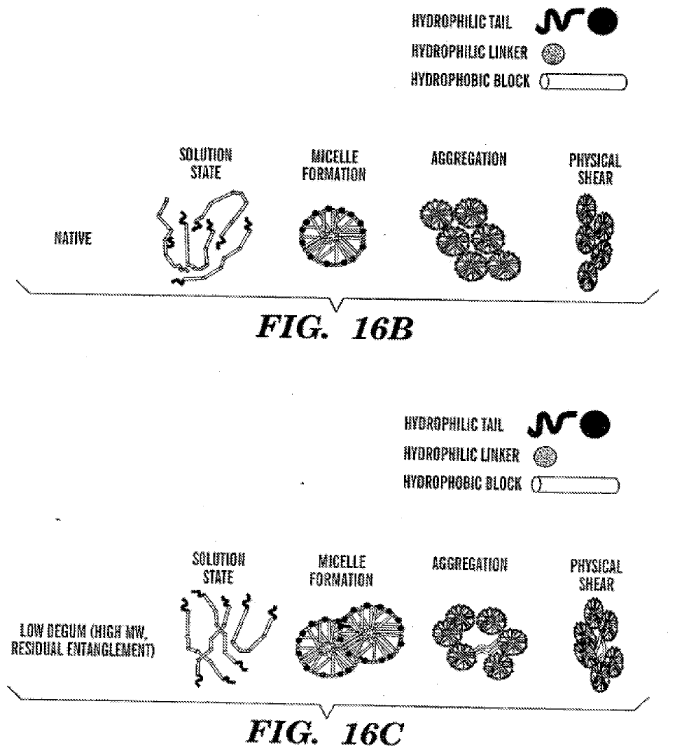

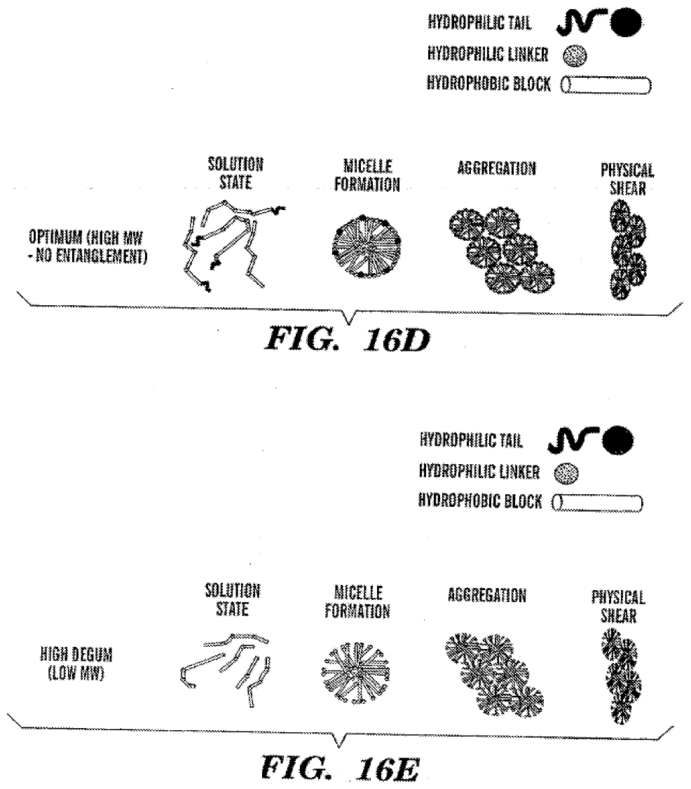

As shown in FIG. 16, for the gently degummed solutions, we suggest that the hydrophilic N and C terminals are largely intact and the fresh solution is close to native in both behavior and make-up. The difference between these regenerated solutions and native is the existence of residual entanglements that were not completely removed during degumming and salvation. Thus, even though the individual protein strands have their native hydrophilic-hydrophobic-hydrophilic tri-block structure, they are prevented from properly folding and assembling into micelles. As the drying and concentration processes progress, the incompletely formed micelles, condense into nanofilaments and crystallize in place. When these structures are subject to shear they initially behave as fully formed micelles, however, as strain is increased, the residual entanglements are engaged, limiting the extensional flow of the molecules. This inhibition of molecular movement then results in stiffening and failure of the material. As the orientation of these entanglements may be off the axis of drawing, both the tensile strength and elasticity of the sample are degraded.

In optimized or standard degumming solutions, a significant number of the N or C terminals have been cleaved during reconstitution, resulting in a number of linker sequences serving as de facto hydrophilic terminals. However, unlike the gently degummed solutions, residual entanglements are substantially broken. As the linker sequences are not as highly hydrophilic as the N or C terminals, the micelle formation is retarded as the propensity for the hydrophilic ends to shield the hydrophobic interior is lessened. From this point, self-assembly progresses as for native fibroin. When these micelles are subject to shear forces they readily flow and elongate. However, due to the fact that they are more loosely associated, they are able to undergo a greater degree of elongation, but are unable to completely engage reducing the tensile strength as compared to native silks.

When degumming times are increased beyond the 15 to 20 minute time frame, significant degradation of the protein chain is experienced. All of the hydrophilic terminals are lost and the linkers are forced to serve as the hydrophilic outer layer during micelle formation. The result is a weakly formed micelle that lacks the highly ordered and layered architecture of native silk. When these materials are sheared the lack of interfacial association between hydrophilic outer layers limits tensile strength, while shortened chain lengths inhibit extensibility.

### Example 3. Exemplary Methods Used for Making Silk Fibers and the Use Thereof

Some studies aim to investigate the effects of degumming on the mechanical properties of native silk fiber. Jiang et al. directly compared the impact on the mechanical behavior of silk fibers that were degummed using different chemical agents that are commonly reported in the literature. Included in the study were distilled water (100° C., 90 min), 0.2 M boracic acid in 0.05 mol/L sodium borate buffer (98° C., 90 min), succinic acid (100° C., 90 min), 8 M urea (80° C., 15 min) and sodium carbonate (80° C., 15 min). Following degumming individual fibers were subjected to tensile testing and stress-strain responses were compared. The results indicated that the chemical composition, temperature and degumming time significantly impacted the strength of silk fibers. In particular the boracic buffer solution at a pH of 9.0 resulted in the highest elastic modulus, ultimate tensile strength and extensibility (Jiang, P., et al. Materials Letters, 2006, 60 (7): 919-925).

In order to assess the inherent variability of silk tensile properties within between cocoons, Zhao et al. unwound cocoons and performed numerous tensile tests on 5 cm segments throughout the length of the resultant fiber. There is significant variability in modulus, tensile strength and extensibility both within the individual fiber that makes up a cocoon and between cocoons spun by different silkworms. While the general material behavior of all fiber segments was comparable, all mechanical properties were shown to vary by nearly an order of magnitude, both within and between the silk fibers (Zhao, H. P., et al., Materials Science and Engineering: C, 2007, 27 (4): 675-683).

In addition to degumming using chemical reagents at elevated temperatures, proteolytic degumming has been proposed as a more environmentally friendly and energy efficient means to remove sericin. Freddi, et al. assessed the effectiveness of 3 different enzymes and found that the GC897-H enzyme was nearly as effective as degumming with alkali soap, with a 25% mass loss as compared with 27% for the soap, as shown in FIG. 6. However, the enzyme degumming can be done at significantly lower temperatures 40-60° C. versus 100° C. and with a lower volume of caustic wastes produced (Freddi, G., et al., Journal of Biotechnology, 2003, 106 (1): 101-112).

While many studies have addressed the impact of the altering the degumming solutions, Ho, et al. used a constant degumming solution and temperature and modulated the duration of fiber immersion. Ho et al. studies silk fibers from tussah, wild type silkworms, which have undergone a degumming in boiling water. They tested native fibers and samples that had been degummed for 15, 30, 45 and 60 minutes and found a significant decrease in mechanical properties with longer degum times. In particular there was a substantial decrease in tensile strength and modulus when the dwell time was increased from 15 to 30 minutes (Ho, M., et al., Applied Surface Science, 2012, 258 (8): 3948-3955). However, Ho does not teach or suggest that substantial amount of sericin can be removed by degumming silk cocoons at boiling temperature for less than 15 minutes, or less than 10 minutes, or less than 5 minutes, while preserving higher molecular weight silk fibroins.

As presented herein, high molecular weight silk fibroin can be produced in milder degumming conditions. In some embodiments, silk fibers based on high molecular weight silk fibroin are produced by electrogelation. Silk electrogelation is a process in which the application of a DC voltage to a silk solution via electrodes causes a conformation change. The resulting gel-like material (“egel”) has many potential applications due to the ability of the meta-stable material to be reversed back to a random coil conformation (silk solution conformation) or further processed into a beta sheet conformation (crystalline, non-reversible conformation). It is known that not all silk solutions form a high-quality egel, depending on how the solution was processed and the material characteristics.

Experiments were conducted to enhance the ability to make quality egel over a range of conditions by utilizing silk fibroin of high molecular weights. Using the standard degumming protocol for all other parameters, silk solution was produced using degumming times of 15, 20, 30, and 60 minutes (boiling milli-Q water). The remaining stages of the solution process (dissolving and dialysis) were then conducted. It was found that the shorter the degumming time, the faster egel forms, producing a higher-quality gel (higher density, larger volume of solution converted to gel, and stiffer). In addition, it was observed that with the lower degumming time solution (higher molecular weights), egel could be formed using lower DC voltage. This is likely due to the density/viscosity of the solution and the improved electrochemical response (conductivity, electron/proton flow). These results are highly significant in terms of the range of conditions over which egel can form, the compatibility of e-gel formation in vivo for biological tissue repair, and generally demonstrate the significant influence of retention of high molecular weight silk on processibility and material properties.

A final modification to improve electrogelation was to utilize higher concentration. By increasing concentration from the standard solution concentration of 7-8% w/v to greater than 25% w/v, silk electrogelation was greatly enhanced.

In order to regenerate silk fibers, an exemplary protocol is described as follows (FIGS. 17A-17E): (a) formation of the silk egel using 10 minute degummed silk solution and platinum electrodes with direct application of DC voltage; (b) heating of the egel to reduce the viscosity and allow ejection from a syringe-based spinneret (c); (d) after fast ejection into a pure water bath; and (e) after drawing of fiber out of water bath. Given higher molecular weight is preserved with shorter degumming time, both egel becomes more effective and the resulting wet-spun regenerated fibers are more robust and stronger. The regenerated silk fibers are shown to be tough enough to tie tight knots in fully dry fiber samples (FIG. 17F). The preservation of long molecular chains due to decreased degumming time is believed to be a key requirement.

### Example 4. Exemplary Methods of Making Silk Foams and the Use Thereof

In order to generate a silk foam, in some embodiments, the silk fibroin solution is poured into a mold and store in a cooler at −10° C. for about 3-5 days. Then it is remove from the cooler and lyophilized for 1 week. Finally, the silk foam-based article is detached from the mold.

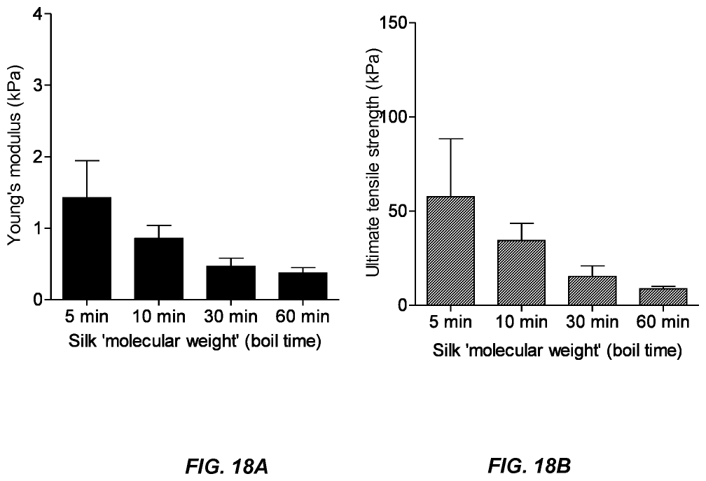

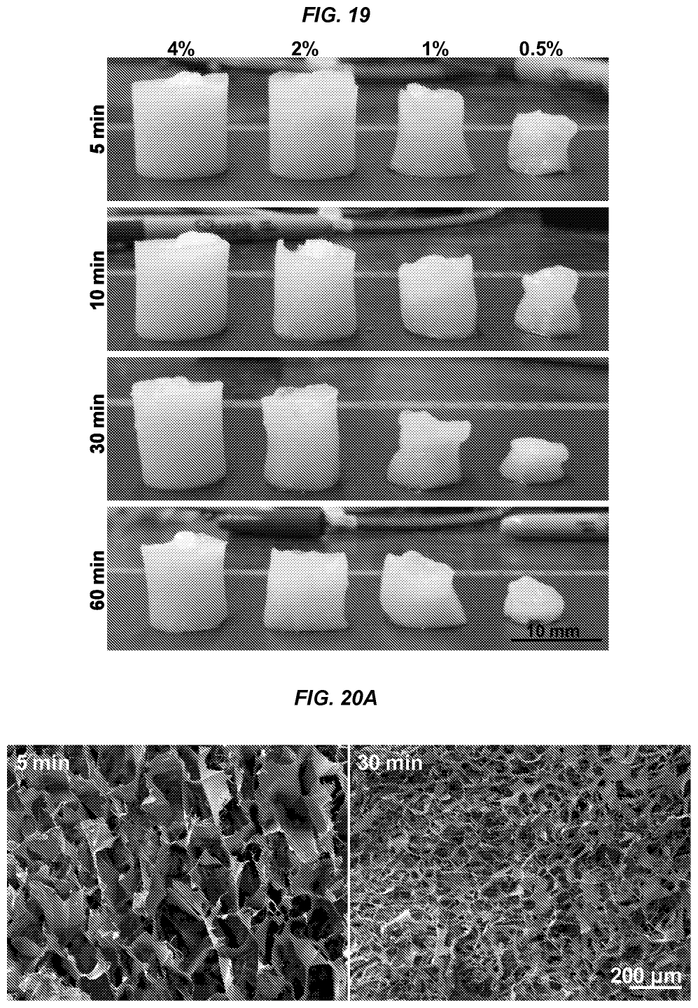

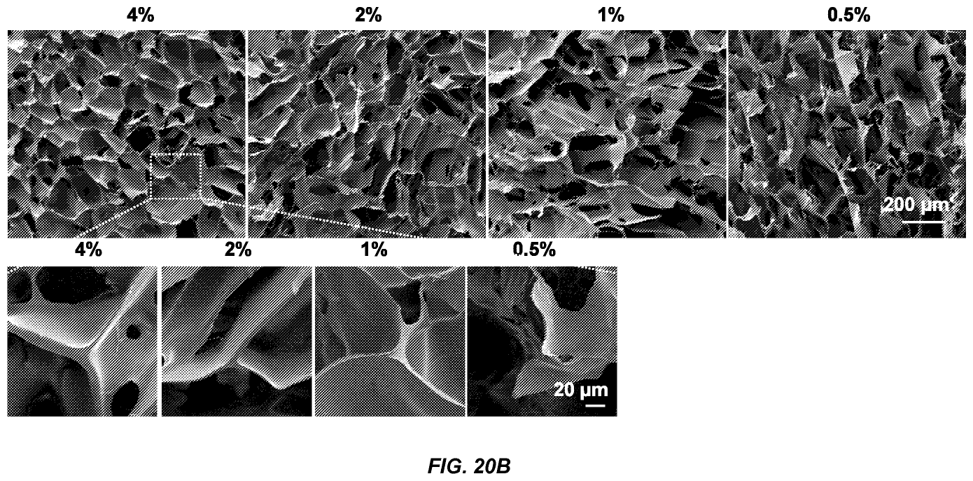

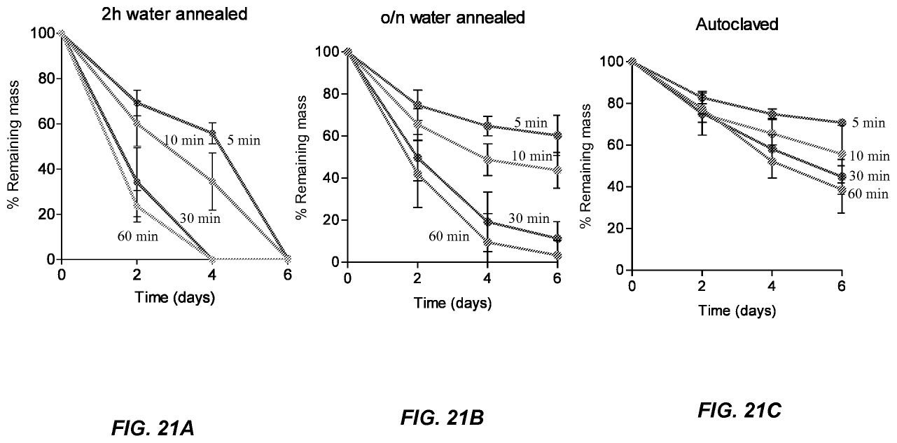

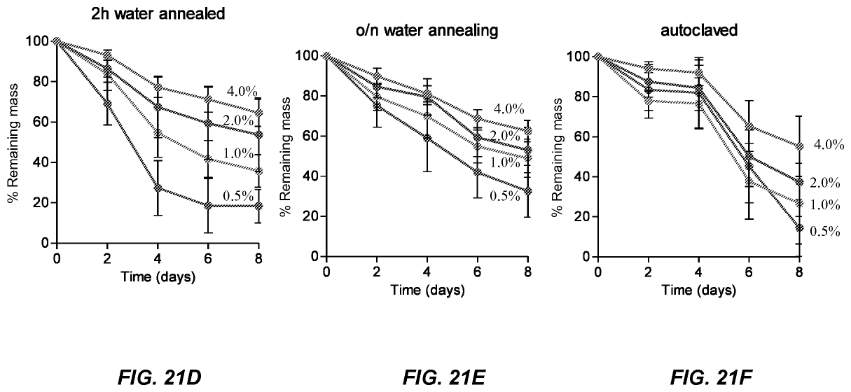

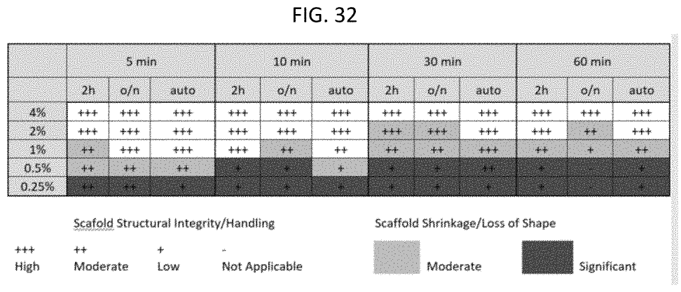

Foams that were created using silk solutions that underwent shorter degumming times had better mechanical performance to traditional cast silk foams. FIG. 18 and FIG. 32 both show that the mechanical properties improve as the boiling time decreases from 60 minutes to 5 minutes. At 0.5% and 1% wt/v, as FIG. 19 shows, all scaffolds underwent shrinkage and some loss of structural integrity. Scaffolds comprising high-molecular-weight fibroin were robust enough to handle and retained their shape relatively well, while those comprising low-molecular-weight fibroin did not maintain their shape and structural integrity. Not wishing to be bound by theory, this difference in mechanical strength can be explained by the presence of lamellas in the scaffolds comprising high-molecular-weight fibroin (FIG. 20A left). FIG. 20B shows the decrease of lamellae wall as the concentration decreases for scaffolds comprising high-molecular-weight fibroin. The wall thickness decrease in turn can explain the degradation kinetics in FIGS. 21A to 21F. Scaffolds of lower concentration degrade faster than those of higher concentration. It is worthwhile to point out that it was not possible to manufacture scaffolds at 0.5% previously because silk fibroin of low molecular weights would render such structures mechanically unstable.

A variety of silk foam-based articles can be created using the protocol described herein. In one embodiment, gold nanoparticles (FIG. 22B) are mixed with the silk fibroin solution before the cooling steps. The gold-doped film can be used as a light-activating heating element for medical purposes and potentially interface with other thermoelectronic components to allow wireless powering of implanted devices.

In some embodiments, three-dimensional constructs can be made using silk foams, as shown in FIGS. 22A & 22C.

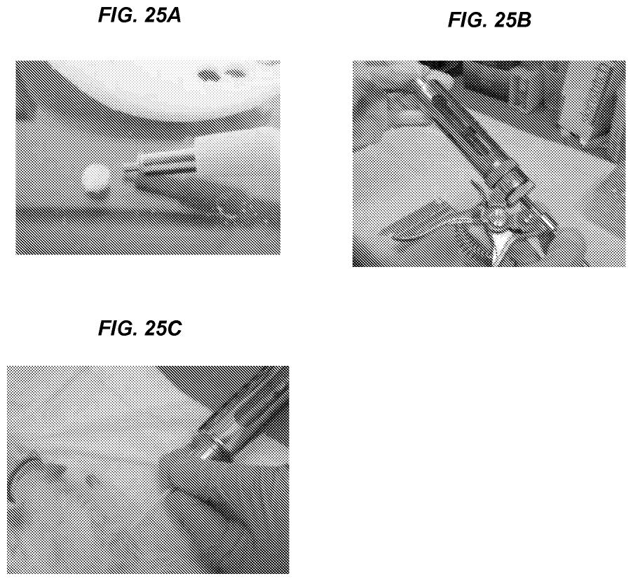

In some embodiments, medical implants can be made using silk foams, as shown in FIGS. 22D and 25A. Along with the control of porosity by silk concentration, good control over the morphology, strength, and toughness of the foams is achievable. Over the range of concentrations tested (1, 2, 3, 4, 5, 6, and ˜7% w/v), the lower concentrations lead to higher porosity and a softer foam geometry.

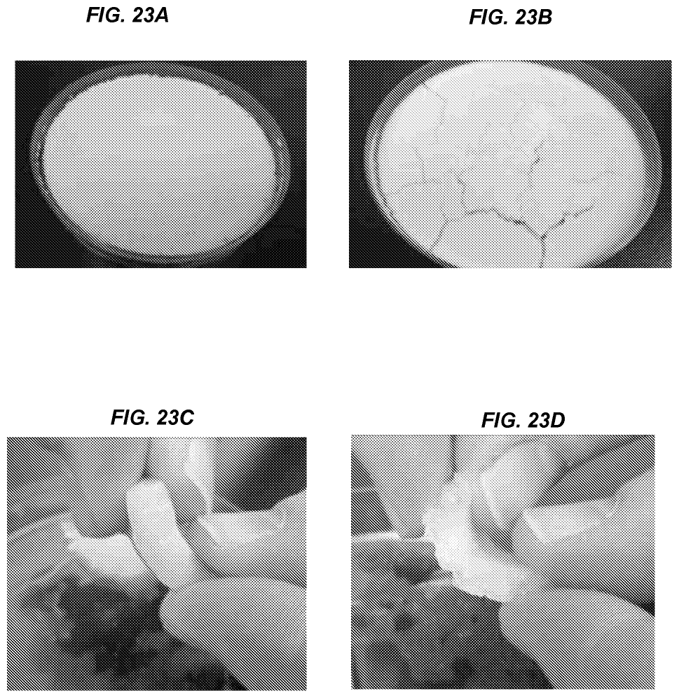

In some embodiments, raw eggs can be stabilized in silk foams. Egg yolk and egg white are mixed with the silk fibroin solution separately before forming the foams. FIGS. 23A-23D show egg yolk and egg white stabilized in a thin foam sheet of silk.

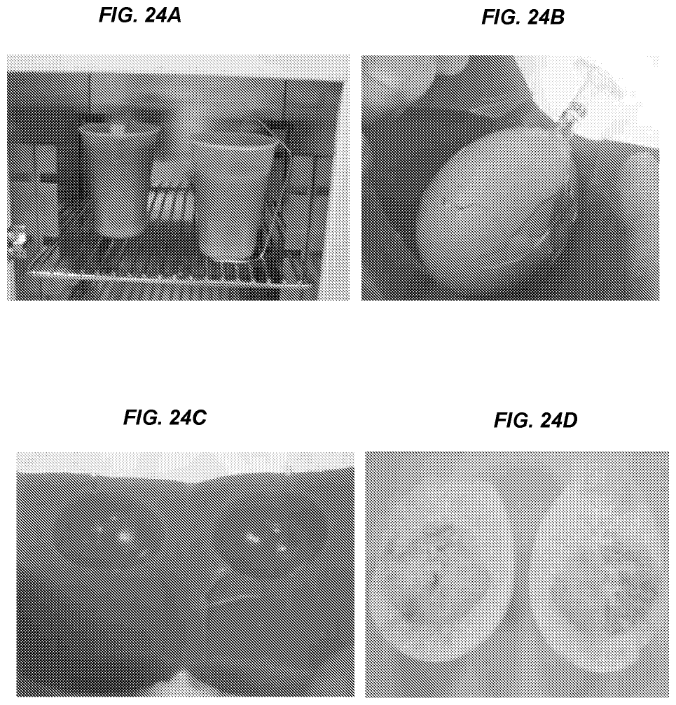

In some embodiments, a solid raw egg/silk integrated construct was fabricated. A hard-boiled egg was suspended in a bath of uncured platinum-cured silicone rubber (DragonSkin from Smooth-On, Inc.). After storing in a 60° C. for 2 hours (FIG. 24A), the fully cured silicone mold was parted with a razor blade and the boiled egg removed (FIG. 24B). The same approach was used to create a mold for the egg yolk, with the exception that a spherical ball (about the expected size of a raw egg yolk) was used as a molding positive (FIG. 24C). The final integrated egg construct is shown in FIG. 24D. The egg material and color was fairly uniform throughout the egg.

In some embodiments, silk foams can be used as subcutaneous implants. Small injectable constructs were excised from the silk foam sheets using a biopsy punch (FIG. 25A). The foams could be loaded in a specially modified syringe injector (FIG. 25B) for subsequent injection into the subcutaneous area of rats (FIG. 25C). The strength and toughness of the foams created using shorter degumming times (and molecular weight preservation), allow them to be initially stored in the injector in a compressed state, squeezed through small-gauge needles, then re-expanded once injected into the subcutaneous area of rats.

### Example 5. Exemplary Methods of Making Silk Tubes and the Use Thereof

In some embodiments, silk fiborin of high molecular weights can be used to form silk tubes. Silk tubes have a wide range of applications including, but not limited to, grafts for tissue engineering and drug delivery. Methods described in the International Application Nos. WO2009/126689 and WO/2009/023615, can be used to form the tubular structure. The contents of those International Application publications are incorporated herein by reference. For example, the tubes can be prepared by using an aqueous gel-spinning approach which allows for precise control of the silk polymer and resultant tube properties. The gel-spinning process comprises that a concentrated silk solution is ejected onto a mandrel such that it evenly coats the surface and maintains a tubular geometry—upon lyophilization and cross-linking, a degradable, porous, and tubular graft material is formed.

In some embodiments, the tubular structure can be formed by contacting a rod of a selected diameter with the aqueous solution of silk fibroin to coat said rod in silk fibroin. The rod can be made of any material that will not strongly stick to the dried silk fibroin. In one embodiment, the rod can be made of stainless steel. In these embodiments, the method can further comprise removing the dried tubular structure from said rod, whereby a tube comprising silk fibroin is made.

Previous reports have shown that the lyophilized gel-spun silk tubes based on silk fibroin of low molecular weights were degrading too slowly and contained too dense a pore architecture to allow for rapid and uniform cellular colonization across the full thickness of the tube walls. Most importantly, this barrier appeared to limit smooth muscle cell activity mainly to the inner-most lumen of the tube and fostered neointimal hyperplasia, a chronic problem with vascular grafts. The invention described herein show that silk fibroin of high molecular weights allows gel spinning at lower concentrations, and the resulting lyophilized gel-spun tubes have larger pores and faster degradation rates.

By improving and refining the gel-spinning process, improved reproducibility of the tubes and added flexibility in processing can form newer and more functional tubes, e.g., designed to act as vessel surrogates. To this end, the inventors have evaluated the effect of modulating the molecular weight of the starting silk solution to form spinning solutions with varying viscosity. For example, once the silk was ejected onto the rotating mandrel, it was found that solutions with a higher molecular weight and thus higher viscosity did not require as high a concentration (% Wt/Vol) to be achieved prior to successful gel-spinning. It was also discovered that the resultant tubes formed from lower concentration solutions had unique pore architectures which scaled in pore size with increasing molecular weight spinning solutions.

The inventors have discovered that the porous structure and/or organization can vary with molecular weight of silk fibroin in the aqueous solution. Accordingly, in some embodiments, an aqueous solution of silk fibroin can be prepared by a method comprising boiling cocoons for at least about 5 mins, at least about 10 mins, at least about 20 mins, at least about 30 mins, at least about 1 hour. The boiling time of silk cocoons generally vary molecular weight of silk fibroin. In some embodiments, the degradation rate of the tubular composition can increase by decreasing boiling time of silk cocoons.

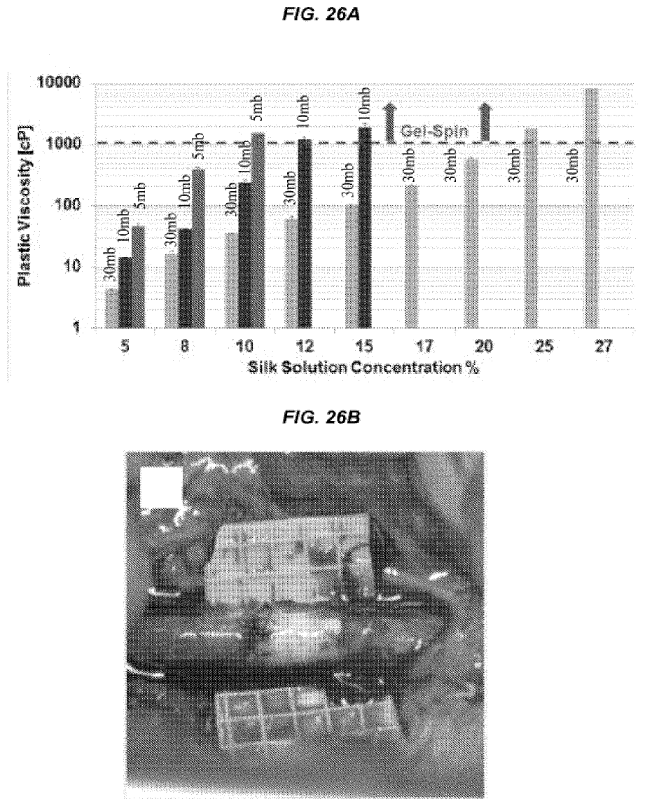

Silk solutions can only be gel-spun when sufficiently concentrated in order for the gel to remain associated with the collection mandrel during rotation (Lovett et al., Biomaterials 2008). Molecular weight and starting solution viscosity were decreased with increased boiling time (Wray L S, Hu X, Gallego J, Georgakoudi I, Omenetto F G, Schmidt D, et al. Effect of processing on silk based biomaterials: reproducibility and biocompatibility. Journal of biomedical materials research Part B, Applied biomaterials. 2011; 99:89-101). The concentrations are desired to be sufficiently increased in order to surpass a minimum viscosity threshold that allowed the resultant gel to remain associated with the collection mandrel during its continuous rotation. However, if the solutions were too heavily concentrated, they were too viscous to eject from the needle used for deposition. As shown in FIG. 26A, increasing boil time decreases viscosity; therefore, less-concentrated solutions are required for gel-spinning solutions from lower boil times. In some embodiments, adequate spinning solutions from the 5 mb, 10 mb, 20 mb, and 30 mb groups can be obtained at concentrations 8-11%, 13-17%, 23-26%, and 30-36%, respectively. In some embodiments, tubes from all boil time solutions can be later lyophilized and then methanol treated for 1 hour in order to induce cross-linking (Lovett et al., Biomaterials 2008) and can be later ethylene oxide sterilized as described previously (Lovett et al., Organogenesis 2010).

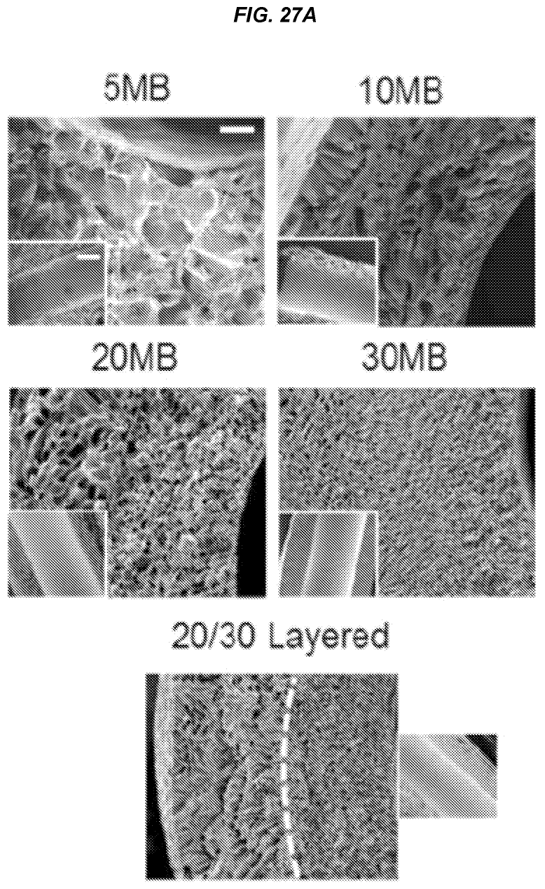

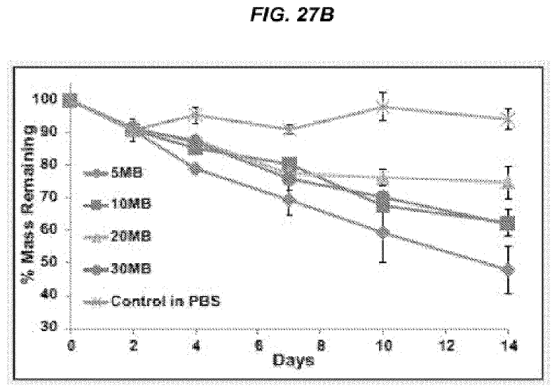

The molecular weight of the silk fibroin solution can be controlled, e.g., by control of silk processing conditions, which can allow for a variety of silk solutions to be gel-spun. In turn, these different silk systems offered differences in structure/properties as shown in FIGS. 27A-27B. In one embodiment, scanning electron microscopy (SEM) can be used to compare various production methods to microstructural properties (e.g., tube pore size and pore interconnectivity) of each graft on the micro- and nano-scale. In some embodiments, tubes contained different pore architectures with pore sizes ranging from ˜200 to ˜20 μm for the 5 mb and 30 mb groups, respectively (see FIG. 27A). Despite these differences in porosity, the inner lumens of the tubes were still noticeably smooth.

To understand how these differentially-porous tube systems can behave in vivo, several groups of tubes with a range of pore architectures (produced by one or more embodiments of the method described herein) can be exposed to a model enzyme that could simulate in vivo degradation kinetics on a relative time scale. Surprisingly, the inventors have discovered that tubes with higher molecular weights, and thus larger pore sizes, degraded faster than their lower molecular weight counterparts. Without wishing to be bound by theory, it is possible that the larger pore sizes allowed for greater fluid transport and enzyme exposure of the grafts, thus facilitating more rapid degradation. In some embodiments, when implanted in vivo, local cells such as smooth muscle and inflammatory cells can colonize the tubular composition described herein (e.g., used as a graft) and enzymatically degrade it faster with larger pore features.

Unexpectedly, when the enzymatic stability of the tubes were compared using a protease digestion assay (see FIG. 27B), it was discovered that the tubes formed using shorter boiling times (with higher molecular weights) were more readily degradable.

The degradation rates of the silk tubes can be further tuned by post-treatments. In some embodiments, the post-treatment can be used to increase beta-sheet content of silk fibroin in the tubular structure. Examples of such post-treatment can include, but are not limited to, methanol or alcohol immersion, water annealing, electric field, pH reduction, mechanical stretching, salt addition, or any combinations thereof.

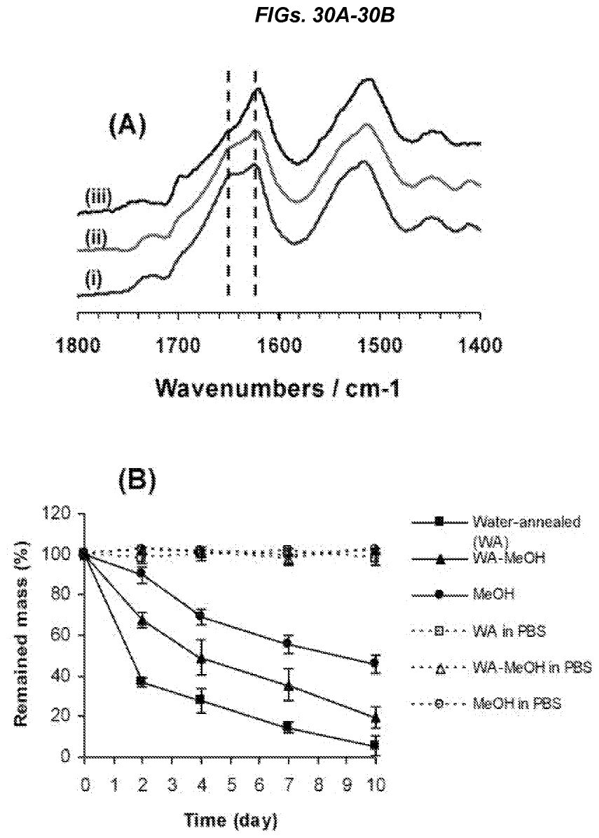

In one embodiment, the post-treatment can comprise water annealing (Hu X, Shmelev K, Sun L, Gil E S, Park S H, Cebe P, et al. Regulation of Silk Material Structure by Temperature-Controlled Water Vapor Annealing. Biomacromolecules. 2011; 12:1686-96; and Jin H J, Park J, Karageorgiou V, Kim U J, Valluzzi R, Cebe P, et al. Water-stable silk films with reduced β-sheet content. Adv Funct Mater. 2005; 15:1241-7). It is shown herein that in some embodiments, the silk solution boiled for 20 minutes (20 mb) was concentrated to 25˜30 w/v % and tubular scaffolds produced by spinning the concentrated silk solutions followed by lyophilization. The tubes were then treated by one of three different methods: 1) water annealed for 5 hours as described in our previous study (Jin et al., 2005), 2) water-annealed for 5 hours followed by 70% MeOH treated for 1 hour, 3) 70% MeOH treated for 1 hour. All tubes were washed in water and air-dried. Secondary structure was confirmed by FTIR and degradation using a standard protease digestion assay, as shown in FIGS. 30A-30B.

In some embodiments, the tubular composition can have an inner lumen diameter of less than 6 mm, less than 5 mm, less than 4 mm, or smaller. In some embodiments, the tubular composition can have an inner lumen diameter of about 0.1 mm to about 6 mm.

The tubular compositions described herein can be used for various applications, e.g., drug delivery or tissue engineering. In some embodiments, the tubular compositions described herein can be implanted in a subject, e.g., a mammalian subject. In some embodiments, the tubular compositions described herein can be used as vascular grafts, e.g., for repair and/or replacement of blood vessels.

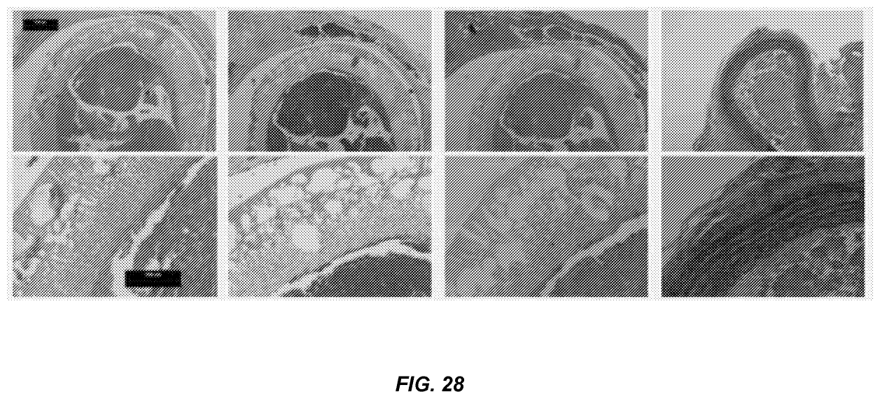

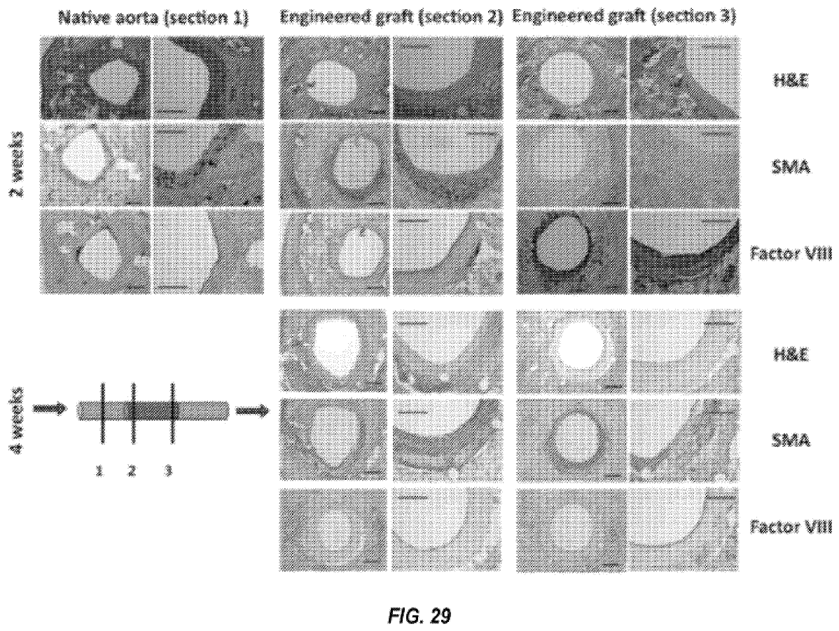

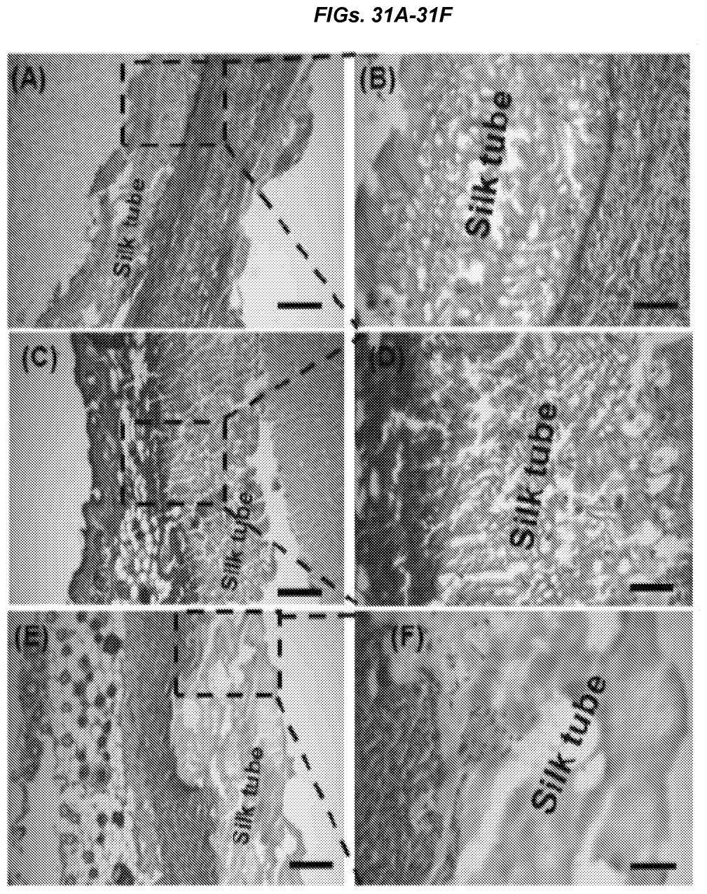

The inventors have shown that in FIG. 28, the lyophilized silk tubes, e.g., at least 1 week after implantation, demonstrated patency and endothelial coverage with minimal inflammatory reactions. The tube systems with variable porosities can behave similarly in vivo, albeit with a slower absolute dissolution kinetics due to the relatively low abundance of broad-specificity enzymes in the blood stream. To evaluate the performance of the tubular compositions produced by the methods described herein, in some embodiments, the tubes can be implanted into the infrarenal abdominal aorta of male 350 g Sprague-Dawley rats via end-to-end anastomosis as previously described (Lovett et al., Organogenesis, 2010). The graft was secured via 9-0 nylon sutures as shown in FIG. 26B. The rat was euthanized at week 1, sample flushed with heparin, immersed in 4% NBF, and paraffin embedded. Cross-sections were made across the tube lumen and sections stained using H&E, Trichrome, and Verhoeffs Elastic Stain. Immunohistochemistry was used to confirm SMA- and Factor VII-positive cells. As shown in FIGS. 31A-31F, in some embodiments, tubular compositions treated by water annealing (WA) or WA followed by methanol soak were the most heavily infiltrated by cells following the 4 weeks in vivo. In particular, the lesser-crosslinked tubes underwent significant remodeling at this time point as revealed by the high magnification images (FIGS. 31B and 31D panels). Conversely, the methanol-treated group showed a nearly uninterrupted pore architecture, suggesting that very little enzymatic degradation had taken place.

Accordingly, some embodiments provided herein relate to small diameter silk tubes, which can be used as a vascular graft, and thus provide a good alternative to existing nondegradable grafts. In some embodiments, methods provided herein produce tubes that can be gel-spun using novel silk formulations with varying molecular weights. Surprisingly, the inventors have discovered that the tubes formed using shorter boiling times (with higher molecular weights) appear to be more readily degradable. Without wishing to be bound by theory, larger pores (formed from a silk solution with shorter boiling time) can be more accessible to fluid interactions with a more interconnected pore network. Conversely, tubes formed with longer boiling-time (e.g., 20 mb) silk solutions can be more enzymatically stable, e.g., due to a balance between silk chain length and accessibility of pore structures. Through the use of a natural biopolymer, silk fibroin, and a gel spinning technique, silk tubes can be produced with precise control over dimensions, micro- and macro-structure, mechanical properties and drug loading and release. Silk fibroin favorably compares to PTFE in terms of thrombogenicity, as demonstrated by untreated silk graft patency over the period of up to 4 weeks, and vascular cell remodeling was observed in rat studies in vivo. Degradation kinetics can be further modified using both control of solution conditions and tube post-processing,

### REFERENCES

Altman, G. H., et al., Silk-based biomaterials. Biomaterials, 2003. 24(3): p. 401-416.
Kinahan, M. E., et al., Tunable Silk: Using Microfluidics to Fabricate Silk Fibers with Controllable Properties. Biomacromolecules, 2011. 12(5): p. 1504-1511.
Marsano, E., et al., Wet spinning of Bombyx mori silk fibroin dissolved in N-methyl morpholine N-oxide and properties of regenerated fibres. International Journal of Biological Macromolecules, 2005. 37(4): p. 179-188.
Wray, L. S., et al., Effect of processing on silk-based biomaterials: Reproducibility and biocompatibility. Journal of Biomedical Materials Research Part B: Applied Biomaterials, 2011. 99B(1): p. 89-101.
Teh, T. K. H. and et al., Optimization of the silk scaffold sericin removal process for retention of silk fibroin protein structure and mechanical properties. Biomedical Materials, 2010. 5(3): p. 035008.
Tao, H., et al., Gold nanoparticle-doped biocompatible silk films as a path to implantable thermo-electrically wireless powering devices. Applied Physics Letters, 2010, 970: p. 123702-1 to 123702-3.
Wang, Y., et al., Stem cell-based tissue engineering with silk biomaterials. Biomaterials, 2006. 27(36): p. 6064-6082.
Inouye, K., et al., Use of Bombyx mori silk fibroin as a substratum for cultivation of animal cells. Journal of Biochemical and Biophysical Methods, 1998. 37(3): p. 159-164.
Cannon, G. M., et al., Silk as a Novel Biomaterial in Bladder Tissue Engineering. Journal of Pediatric Urology, 2010. 6: p. S82.
Wenk, E., et al., Silk fibroin spheres as a platform for controlled drug delivery. Journal of Controlled Release, 2008. 132(1): p. 26-34.
Meinel, L. and D. L. Kaplan, Silk constructs for delivery of muskuloskeletal therapeutics. Advanced drug delivery reviews, 2012.
Hofmann, S., et al., Silk fibroin as an organic polymer for controlled drug delivery. Journal of Controlled Release, 2006. 111(1): p. 219-227.
Omenetto, F. G. and D. L. Kaplan, New opportunities for an ancient material. Science, 2010. 329(5991): p. 528-531.
Demura, M. and T. Asakura, Immobilization of glucose oxidase with Bombyx mori silk fibroin by only stretching treatment and its application to glucose sensor. Biotechnology and Bioengineering, 1989. 33(5): p. 598-603.
Amsden, J. J., et al. Silk fibroin biosensor based on imprinted periodic nanostructures. 2009. IEEE.
Mita, K., S. Ichimura, and T. C. James, Highly repetitive structure and its organization of the silk fibroin gene. Journal of molecular evolution, 1994. 38(6): p. 583-592.
Fu, C., Z. Shao, and V. Fritz, Animal silks: their structures, properties and artificial production. Chemical Communications, 2009(43): p. 6515-6529.
Vepari, C. and D. L. Kaplan, Silk as a biomaterial. Progress in Polymer Science, 2007. 32(8-9): p. 991-1007.
Sugihara, A., et al., Promotive effects of a silk film on epidermal recovery from full-thickness skin wounds (44552). Experimental Biology and Medicine, 2000. 225(1): p. 58-64.
Yeo, J. H., et al., The effects of PVA/Chitosan/Fibroin (PCF)-blended spongy sheets on wound healing in rats. Biological and Pharmaceutical Bulletin, 2000. 23(10): p. 1220-1223.
Meinel, L., et al., Bone tissue engineering using human mesenchymal stem cells: Effects of scaffold material and medium flow. Annals of Biomedical Engineering, 2004. 32(1): p. 112-122.
Meinel, L., et al., Silk implants for the healing of critical size bone defects. Bone, 2005. 37(5): p. 688-698.
Kim, H. J., et al., Influence of macroporous protein scaffolds on bone tissue engineering from bone marrow stem cells. Biomaterials, 2005. 26(21): p. 4442-4452.
Meinel, L., et al., Silk based biomaterials to heal critical sized femur defects. Bone, 2006. 39(4): p. 922-931.
Meinel, L., et al., Osteogenesis by human mesenchymal stem cells cultured on silk biomaterials: Comparison of adenovirus mediated gene transfer and protein delivery of BMP-2. Biomaterials, 2006. 27(28): p. 4993-5002.
Karageorgiou, V., et al., Porous silk fibroin 3-D scaffolds for delivery of bone morphogenetic protein-2 in vitro and in vivo. Journal of Biomedical Materials Research—Part A, 2006. 78(2): p. 324-334.
Marolt, D., et al., Bone and cartilage tissue constructs grown using human bone marrow stromal cells, silk scaffolds and rotating bioreactors. Biomaterials, 2006. 27(36): p. 6138-6149.
Karageorgiou, V., et al., Bone morphogenetic protein-2 decorated silk fibroin films induce osteogenic differentiation of human bone marrow stromal cells. Journal of Biomedical Materials Research—Part A, 2004. 71(3): p. 528-537.
Sofia, S., et al., Functionalized silk-based biomaterials for bone formation. Journal of Biomedical Materials Research, 2001. 54(1): p. 139-148.
Kino, R., et al., Deposition of bone-like apatite on modified silk fibroin films from simulated body fluid. Journal of Applied Polymer Science, 2006. 99(5): p. 2822-2830.
Motta, A., et al., Fibroin hydrogels for biomedical applications: Preparation, characterization and in vitro cell culture studies. Journal of Biomaterials Science, Polymer Edition, 2004. 15(7): p. 851-864.
Fini, M., et al., The healing of confined critical size cancellous defects in the presence of silk fibroin hydrogel. Biomaterials, 2005. 26(17): p. 3527-3536.
Li, C., et al., Electrospun silk-BMP-2 scaffolds for bone tissue engineering. Biomaterials, 2006. 27(16): p. 3115-3124.
Kim, K. H., et al., Biological efficacy of silk fibroin nanofiber membranes for guided bone regeneration. Journal of Biotechnology, 2005. 120(3): p. 327-339.
Meinel, L., et al., Engineering cartilage-like tissue using human mesenchymal stem cells and silk protein scaffolds. Biotechnology and Bioengineering, 2004. 88(3): p. 379-391.
Wang, Y., et al., Cartilage tissue engineering with silk scaffolds and human articular chondrocytes. Biomaterials, 2006. 27(25): p. 4434-4442.
Morita, Y., et al., Visco-elastic properties of cartilage tissue regenerated with fibroin sponge. Bio-Medical Materials and Engineering, 2002. 12(3): p. 291-298.
Morita, Y., et al., Frictional properties of regenerated cartilage in vitro. Journal of Biomechanics, 2006. 39(1): p. 103-109.
Aoki, H., et al., Culture of chondrocytes in fibroin-hydrogel sponge. Bio-Medical Materials and Engineering, 2003. 13(4): p. 309-316.
Chen, J., et al., Human bone marrow stromal cell and ligament fibroblast responses on RGD-modified silk fibers. Journal of Biomedical Materials Research—Part A, 2003. 67(2): p. 559-570.
Altman, G. H., et al., Silk matrix for tissue engineered anterior cruciate ligaments. Biomaterials, 2002. 23(20): p. 4131-4141.
Moreau, J. E., et al., Sequential growth factor application in bone marrow stromal cell ligament engineering. Tissue Engineering, 2005. 11(11-12): p. 1887-1897.
Kardestuncer, T., et al., RGD-tethered silk substrate stimulates the differentiation of human tendon cells. Clinical Orthopaedics and Related Research, 2006 (448): p. 234-239.
Hu, K., et al., Biocompatible fibroin blended films with recombinant human-like collagen for hepatic tissue engineering. Journal of bioactive and compatible polymers, 2006. 21(1): p. 23-37.
Dal Pra, I., et al., De novo engineering of reticular connective tissue in vivo by silk fibroin nonwoven materials. Biomaterials, 2005. 26(14): p. 1987-1999.
Unger, R. E., et al., Endothelialization of a non-woven silk fibroin net for use in tissue engineering: Growth and gene regulation of human endothelial cells. Biomaterials, 2004. 25(21): p. 5137-5146.
Fuchs, S., et al., Outgrowth endothelial cells isolated and expanded from human peripheral blood progenitor cells as a potential source of autologous cells for endothelialization of silk fibroin biomaterials. Biomaterials, 2006. 27(31): p. 5399-5408.
Lee, K. Y., et al., Effect of surface properties on the antithrombogenicity of silk fibroin/S-carboxymethyl kerateine blend films. Journal of Biomaterials Science, Polymer Edition, 1998. 9(9): p. 905-914.
Holland, C., et al., Natural and unnatural silks. Polymer, 2007. 48(12): p. 3388-3392.
Yin, J., et al., Enhancing the Toughness of Regenerated Silk Fibroin Film through Uniaxial Extension. Biomacromolecules, 2010. 11(11): p. 2890-2895.
Lee, Y. W., Silk reeling and testing manual. Vol. 136. 1999: Food & Agriculture Organization of the UN (FAO).
Zhou, C. Z., et al., Silk fibroin: structural implications of a remarkable amino acid sequence. Proteins: Structure, Function, and Bioinformatics, 2001. 44(2): p. 119-122.
Murphy, A. R. and D. L. Kaplan, Biomedical applications of chemically modified silk fibroin. Journal of Materials Chemistry, 2009. 19(36): p. 6443-6450.
Jin, H. J. and D. L. Kaplan, Mechanism of silk processing in insects and spiders. Nature, 2003. 424(6952): p. 1057-1061.
Holland, C., et al., Polymer Fibers: Silk and Synthetic Polymers: Reconciling 100 Degrees of Separation (Adv. Mater. 1/2012). Advanced Materials, 2012. 24(1): p. 104-104.
Takei, F., et al., Further evidence for importance of the subunit combination of silk fibroin in its efficient secretion from the posterior silk gland cells. The Journal of cell biology, 1987. 105(1): p. 175-180.
Tanaka, K., K. Mori, and S. Mizuno, Immunological identification of the major disulfide-linked light component of silk fibroin. Journal of biochemistry, 1993. 114(1): p. 1-4.
Tanaka, K., et al., Determination of the site of disulfide linkage between heavy and light chains of silk fibroin produced by Bombyx mori. Biochimica et Biophysica Acta (BBA)-Protein Structure and Molecular Enzymology, 1999. 1432(1): p. 92-103.
Takasu, Y., H. Yamada, and K. Tsubouchi, Isolation of Three Main Sericin Components from the Cocoon of the Silkworm, Bombyx mori. Bioscience, Biotechnology, and Biochemistry, 2002. 66(12): p. 2715-2718.
Rockwood, D. N., et al., Materials fabrication from Bombyx mori silk fibroin. Nat. Protocols, 2011. 6(10): p. 1612-1631.
Panilaitis, B., et al., Macrophage responses to silk. Biomaterials, 2003. 24(18): p. 3079-3085.
Altman, G. H. C., Jingsong; Horan, Rebecca; Horan, David, Immunoneutral Silk-Fiber-Based Medical Devices, 2004, Tissue Regeneration, Inc.: United States. p. 45.
Yamada, H., et al., Preparation of undegraded native molecular fibroin solution from silkworm cocoons. Materials Science and Engineering: C, 2001. 14(1-2): p. 41-46.
Jiang, P., et al., Tensile behavior and morphology of differently degummed silkworm (Bombyx mori) cocoon silk fibres. Materials Letters, 2006. 60(7): p. 919-925.
Zhao, H. P., X. Q. Feng, and H. J. Shi, Variability in mechanical properties of Bombyx mori silk. Materials Science and Engineering: C, 2007. 27(4): p. 675-683.
Freddi, G., R. Mossotti, and R. Innocenti, Degumming of silk fabric with several proteases. Journal of Biotechnology, 2003. 106(1): p. 101-112.
Ho, M., H. Wang, and K. Lau, Effect of degumming time on silkworm silk fibre for biodegradable polymer composites. Applied Surface Science, 2012. 258(8): p. 3948-3955.
Zhang, c., et al., Flexibility regeneration of silk fibroin in vitro. Biomacromolecules, 2012.
Zhou, G., et al., Silk Fibers Extruded Artificially from Aqueous Solutions of Regenerated Bombyx mori Silk Fibroin are Tougher than their Natural Counterparts. Advanced Materials, 2009. 21(3): p. 366-370.
Wei, W., et al., Bio-inspired capillary dry spinning of regenerated silk fibroin aqueous solution. Materials Science and Engineering: C, 2011. 31(7): p. 1602-1608.
Phillips, D. M., et al., Regenerated silk fiber wet spinning from an ionic liquid solution. Journal of Materials Chemistry, 2005. 15(39): p. 4206-4208.
Yan, J., et al., Wet-Spinning of Regenerated Silk Fiber from Aqueous Silk Fibroin Solution: Discussion of Spinning Parameters. Biomacromolecules, 2009. 11(1): p. 1-5.
Sasaki, T. and H. Noda, Studies on silk fibroin of Bombyx mori directly extracted from the silk gland: I. Molecular weight determination in guanidine hydrochloride or urea solutions. Biochimica et Biophysica Acta (BBA)—Protein Structure, 1973. 310(1): p. 76-90.
Zainuddin, T. T., et al., The behavior of aged regenerated Bombyx mori silk fibroin solutions studied by H NMR and rheology. Biomaterials, 2008. 29(32): p. 4268-4274.
Lu, Q., et al., Water-insoluble silk films with silk I structure. Acta Biomaterialia, 2010. 6(4): p. 1380-1387.
Lu, Q., et al., Silk Self-Assembly Mechanisms and Control—From Thermodynamics to Kinetics. Biomacromolecules, 2012.
Hwang, S. W., et al., A Physically Transient Form of Silicon Electronics. Science 2012, 337(6102): 1640-1644.
Mitchell S L, and Niklason L E, 2003, Cardiovasc Pathol 12(2):59-64.
Baguneid M S, et al., 2006, Br J Surg. 93(3):282-290.
Lovett M, Eng G, Kluge J A, Cannizzaro C, Vunjak-Novakovic G, Kaplan D L. Tubular silk scaffolds for small diameter vascular grafts. Organogenesis. 2010; 6:217-24.
Pritchard et al., Macromol. Biosci. 2013.
Lovett et al., Biomaterials 2008.
Hu X, Shmelev K, Sun L, Gil E S, Park S H, Cebe P, et al. Regulation of Silk Material Structure by Temperature-Controlled Water Vapor Annealing. Biomacromolecules. 2011; 12:1686-96.
Jin H J, Park J, Karageorgiou V, Kim U J, Valluzzi R, Cebe P, et al. Water-stable silk films with reduced β-sheet content. Adv Funct Mater. 2005; 15:1241-7.

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1-35. (canceled)

36. A method of producing a silk fibroin article comprising
(i) providing a composition comprising reconstituted silk fibroin having an average molecular weight ranging from 40 kDa to 290 kDa, wherein the composition is substantially sericin-free, wherein no more than 50% by weight of the silk fibroin has a molecular weight of less than 100 kDa;
(ii) forming the silk fibroin article from the composition; and
(iii) subjecting the silk fibroin article to a post-treatment.

37. The method of claim 36, wherein the silk fibroin article is formed from the composition by a process selected from the group consisting of gel spinning, lyophilization, casting, molding, electrospinning, machining, wet-spinning, dry-spinning, milling, spraying, phase separation, template-assisted assembly, rolling, compaction, and any combinations thereof.

38. The method of claim 36, wherein the composition is a solution or powder.

39. The method of claim 36, wherein the post-treatment comprises steam drawing.

40. The method of claim 36, wherein the post-treatment induces a conformational change in the silk fibroin in the article.

41. The method of claim 40, wherein said inducing conformational change comprises one or more of lyophilization, water annealing, water vapor annealing, alcohol immersion, sonication, shear stress, electrogelation, pH reduction, salt addition, air-drying, electrospinning, stretching, or any combination thereof.

42. The method of claim 36, wherein the silk fibroin article is in a form selected from the group consisting of a film, a sheet, a gel or hydrogel, a mesh, a mat, a non-woven mat, a fabric, a scaffold, a tube, a slab or block, a fiber, a particle, powder, a 3-dimensional construct, an implant, a foam or a sponge, a needle, a lyophilized article, and any combinations thereof.

43. The method of claim 36, wherein the silk fibroin article further comprises an additive.

44. The method of claim 43, wherein the additive is selected from the group consisting of biocompatible polymers; plasticizers; stimulus-responsive agents; small organic or inorganic molecules; saccharides; oligosaccharides; polysaccharides; peptides, proteins, and peptide analogs and derivatives; peptidomimetics; antibodies and antigen binding fragments thereof; nucleic acids; nucleic acid analogs and derivatives; glycogens or other sugars; immunogens; antigens; an extract made from a biological material; animal tissues; animal tissues; naturally occurring or synthetic compositions; and any combinations thereof.

45. The method of claim 43, wherein the additive is in a form selected from the group consisting of a particle, a fiber, a film, a gel, a tube, a mesh, a mat, a non-woven mat, a powder, and any combinations thereof.

46. The method of claim 45, wherein the particle is a nanoparticle or a microparticle.

47. The method of claim 43, wherein the additive comprises a calcium phosphate (CaP) material.

48. The method of claim 43, wherein the additive comprises a silk material selected from the group consisting of silk particles, silk fibers, micro-sized silk fibers, and unprocessed silk fibers.

49. The method of claim 36, wherein the composition further comprises an active agent.

50. The method of claim 43, wherein the composition comprises from about 0.1% (w/w) to about 99% (w/w) of the additive.

51. The method of claim 36, wherein the silk fibroin article is a film.

52. The method of claim 36, wherein the post-treatment is water vapor annealing, air-drying, or a combination thereof.

53. A method of producing a silk fibroin article comprising
(i) providing a composition comprising silk fibroin having an average molecular weight ranging from 40 kDa to 290 kDa, wherein the composition is substantially sericin-free, wherein no more than 50% by weight of the silk fibroin has a molecular weight of less than 100 kDa; and
(ii) forming the silk fibroin article from the composition;
(iii) wherein the silk fibroin article further comprises an additive, wherein:
(a) the additive is in the form of a nanoparticle or a microparticle, or
(b) the additive comprises a calcium phosphate (CaP) material, or
the additive comprises a silk material selected from the group consisting of silk particles, silk fibers, micro-sized silk fibers, and unprocessed silk fibers.

54. The method of claim 53, wherein the silk fibroin article is a film.

55. The method of claim 53, wherein the post-treatment induces a conformational change in the silk fibroin in the article.
